(19) United States
(12) Patent Application Publication
(10) Pub. No.: US 2026/0026504 A1
(43) Pub. Date: Jan. 29, 2026

(54) COMPOUND CONTAINING PYRAZOLE STRUCTURE, APPLICATION, AND INSECTICIDE

(71) Applicant: Anhui Shengfeng Biochemical Co., Ltd, Hefei, Anhui (CN)

(72) Inventors: Guangfu YANG, Wuhan (CN); Wei HUANG, Wuhan (CN); Zhepeng MA, Wuhan (CN); Zhenyu WANG, Wuhan (CN); Jinzhen WANG, Wuhan (CN); Jianyou LI, Hefei (CN)

(73) Assignee: Anhui Shengfeng Biochemical Co., Ltd, Hefei, Anhui (CN)

(21) Appl. No.: 18/992,340

(22) PCT Filed: Jul. 13, 2023
(86) PCT No.: PCT/CN2023/107234

§ 371 Date: Jan. 8, 2025

(30) Foreign Application Priority Data
Jul. 13, 2022 (CN) 202210826763.6

(51) Int. Cl. A01N 43/80; A01P 7/04; C07D 413/12

(52) U.S. Cl. CPC A01N 43/80; A01P 7/04; C07D 413/12

(57) ABSTRACT

Pesticides and insecticides are disclosed. A compound containing a pyrazole structure, an application thereof, and an insecticide containing the compound are disclosed. The compound containing the pyrazolc structure has a structure of the following Formula (1). The compound can, at low concentrations, successfully kill insects, mites, nematodes and piercing-sucking insects.

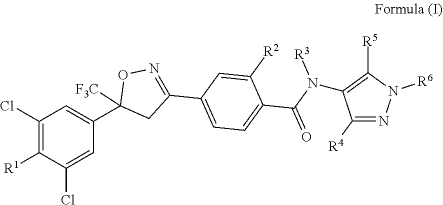

### CROSS REFERENCE TO RELATED APPLICATIONS

This application is a National Stage of International Application No. PCT/CN2023/107234 filed Jul. 13, 2023, claiming priority based on Chinese patent application 202210826763.6, filed on Jul. 13, 2022, the contents of which are incorporated herein by reference.

### FIELD OF THE INVENTION

The present invention relates to the field of pesticides and insecticides, and particularly relates to compound containing pyrazole structure, application, and insecticide.

### BACKGROUND OF THE INVENTION

γ-aminobutyric acid (GABA) is an important neurotransmitter in animal body, is mainly present in central nervous system, and is converted from glutamate by enzymatic metabolism in the body.

As an inhibitory neurotransmitter, GABA is released from the presynaptic membrane of a nerve cell, reaches the postsynaptic membrane through a synaptic cleft, and is specifically combined with a GABA receptor on the postsynaptic membrane to promote chloride ion channels on cell membranes to be opened, chloride ions outside the membranes enter cells to generate a hyperpolarization phenomenon, so that the nerve cell is inhibited from exciting, and the physiological action is played.

Isoxazolines are a new class of pesticides that act on specific sites of ionic GABA receptors. The compound noncompetitive blocks the inflow of chlorine ions of nerve cells, interferes with the normal function of the central nervous system of insects, and enables the nerve excitation not to be normally inhibited, thereby leading the insects to die.

The isoxazoline compound has the characteristics of broad spectrum, high activity, high selectivity and the like, and has good biological activity on agricultural pests such as Hemiptera, Thysanoptera, Diptera, Lepidoptera, mites and the like.

Therefore, the isoxazoline compound is a pesticide with great research value and development prospect.

In 2005, Nissan Chemical Corporation disclosed that isoxazolines had insecticidal properties in WO2005085216A1. The representative substances are a compound K1 and a compound K2, and the structures are as follows, and the substances are mainly used for controlling lepidoptera pests in crops.

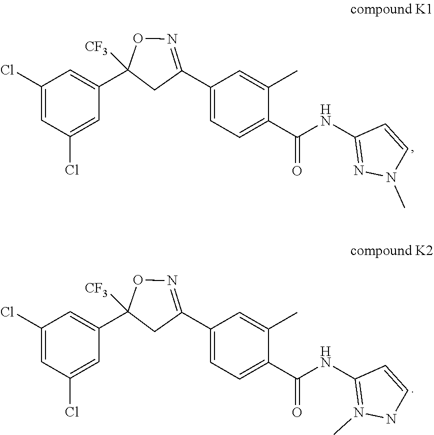

Subsequently, WO2007026965, WO2008108448, WO2011104089, WO2019243262, WO2012007426 and other prior arts disclose a series of compounds containing isoxazoline structures with insecticidal activity on the basis of the prior art. However, the insecticidal or acaricidal activity of the above compounds is still insufficient in practical application, and the activity of the compounds to other target organisms besides whiteflies is not satisfactory.

### DESCRIPTION OF THE INVENTION

The invention aims to provide a novel pyrazole structure-containing compound which can successfully kill insects, mites, nematodes and piercing-sucking insects at low concentrations.

The inventors of the present invention have unexpectedly found in their studies that a novel compound obtained by shifting the 5-aminopyrazole structure of prior art isoxazolines to 4-aminopyrazole and introducing or not introducing characteristic substituents at the 1, 3, 5-positions has excellent inhibitory activity against pests. Meanwhile, the novel compound has good safety on aquatic organisms such as fish, daphnia, algae and the like. That is, the present invention can provide a novel isoxazoline pesticide having both environmental friendliness and high inhibitory activity against pests. In view of the above, the inventor provides the scheme of the invention.

In order to achieve the above object, a first aspect of the present invention provides a compound having a pyrazole structure, or an agrochemically acceptable salt, hydrate and solvate thereof, the compound having a structure represented by formula (I):

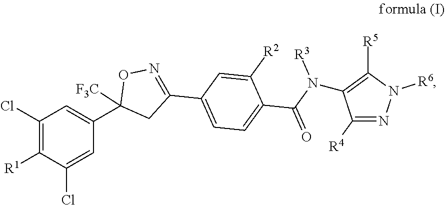

wherein, in the formula (I),
R¹ is selected from H, halogen, at least one halogen-substituted C₁₋₆ alkyl, at least one halogen-substituted C₁₋₆ alkoxy;
R² is selected from C₁₋₁₂ alkyl, C₁₋₁₂ alkoxy, halogen, at least one halogen-substituted C₁₋₆ alkyl, at least one halogen-substituted C₁₋₆ alkoxy and cyano;
R³ is selected from H, C₁₋₁₂ alkyl, C₁₋₁₂ alkoxy, C₃₋₁₂ cycloalkyl, —CO—R₃₁, —CO—CH₂—R₃₁; R₃₁ is selected from C₁₋₁₂ alkyl, C₃₋₁₂ cycloalkyl and C₁₋₁₂ alkoxy;
R⁴, R⁶ are respectively and independently selected from H, C₁₋₁₂ alkyl, C₁₋₁₂ alkoxy, halogen, at least one halogen-substituted C₁₋₆ alkyl and at least one halogen-substituted C₁₋₆ alkoxy;
R⁶ is selected from the group consisting of H, C₁₋₁₂ alkyl unsubstituted or substituted by at least one group from combination A, C₁₋₁₂ alkoxy unsubstituted or substituted by at least one group from combination A, C₃₋₁₂ cycloalkyl unsubstituted or substituted by at least one group from combination A, phenyl unsubstituted or substituted by at least one group from combination A, C₃₋₁₂ cycloalkyl containing at least one heteroatom as a ring-forming atom, C₂₋₁₂ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂, R₆₁ is selected from the group consisting of C₃₋₁₂ cycloalkyl, C₃₋₁₂ cycloalkyl containing at least one heteroatom as a ring-forming atom, phenyl, R₆₂ is selected from the group consisting of C₁₋₁₂ alkyl, phenyl unsubstituted or substituted by at least one of group A, C₃₋₁₂ cycloalkyl, C₃₋₁₂ cycloalkyl containing at least one heteroatom as a ring-forming atom; R₆₃, R₆₄ are independently selected from C₁₋₁₂ alkyl; each of the heteroatoms is independently at least one selected from N, O, S; each n is independently 1 or 2;
the combination A consists of hydroxyl, halogen, C₁₋₁₂ alkyl, C₁₋₁₂ alkoxy and trimethylsilyl.

In a second aspect, the present invention provides the use of the compound containing a pyrazole structure according to the first aspect, or an agrochemically acceptable salt, hydrate, and solvate thereof, for at least one of insect killing, mite killing, nematode killing, and piercing-sucking insects killing.

A third aspect of the present invention provides the use of the aforementioned pyrazole structure-containing compound or an agrochemically acceptable salt, hydrate and solvate thereof as an agrochemical insecticide.

A fourth aspect of the present invention provides an insecticide containing a pyrazole structure or an agrochemically acceptable salt, hydrate and solvate thereof as an active ingredient.

The compound or the agrochemically acceptable salt, hydrate and solvate of the compound has excellent control effect on pests such as Plutella xylostella, Spodoptera frugiperda, Spodoptera litura, Myzus persicae, Spodoptera exigua, Ostrinia furnacalis, Helicoverpa armigera, Chilo suppressalis, Cnaphalocrocis medinalis, Frankliniella occidentalis, flea beetle and the like, and has good market development prospect.

The compound or the agrochemically acceptable salt, hydrate and solvate thereof provided by the invention can also effectively control mutant pests which have resistance to the existing insecticide, and has important significance for developing novel insecticides without cross resistance.

### DETAILED DESCRIPTION

The endpoints of the ranges and any values disclosed herein are not limited to the precise range or value, and these ranges or values should be understood to encompass values close to these ranges or values. For numerical ranges, each range between its endpoints and individual point values, and each individual point value can be combined with each other to give one or more new numerical ranges, and such numerical ranges should be construed as specifically disclosed herein.

Some exemplary explanations are provided below for the partial groups of the present invention, and the unrecited parts are explained with reference to the following exemplary explanations without particular description.

“halogen” means fluorine, chlorine, bromine, iodine.

“C₁₋₆ alkyl” refers to a straight or branched chain alkyl group having a total number of carbon atoms of 1, 2, 3, 4, 5 or 6. Such as methyl, ethyl, n-propyl, isopropyl, n-butyl, isobutyl, tert-butyl, and the like. “C₁₋₈ alkyl”, “C₁₋₁₀ alkyl”, “C₁₋₁₂ alkyl” have similar definitions, except that the total number of carbon atoms is different.

“C₁₋₆ alkoxy” means a straight or branched chain alkoxy group having a total number of carbon atoms of 1, 2, 3, 4, 5 or 6. Such as methoxy, ethoxy, n-propoxy, isopropoxy, n-butoxy, isobutoxy, tert-butoxy and the like. “C₁₋₈ alkoxy”, “C₁₋₁₀ alkoxy” and “C₁₋₁₂ alkoxy” have similar definitions, only differing in the total number of carbon atoms.

“at least one halogen-substituted C₁₋₆ alkyl” means that at least one H atom in the “C₁₋₆ alkyl” is substituted by halogen, and the substituted halogen may be one or more. “at least one halogen-substituted C₁₋₆ alkoxy” has a similar definition to that of “C₁₋₆ alkyl” merely replacing it with “C₁₋₆ alkoxy”.

“C₃₋₁₀ cycloalkyl” refers to cycloalkyl having 3-10 carbon atoms in total, and the ring-forming atoms are all C atoms, and may be, for example, 3, 4, 5, 6, 7, 8, 9, 10 ring-forming carbon atoms in total, and any position on the “C₃₋₁₀ cycloalkyl” which may be substituted is directly connected to the parent nucleus. “C₃₋₆ cycloalkyl” and “C₃₋₁₂ cycloalkyl” have similar definitions, except for the total number of carbon atoms.

“C₁₋₁₂ alkyl unsubstituted or substituted by at least one group from combination A” means that there is no substituent in “C₁₋₁₂ alkyl” or that it is substituted by one or more groups from combination A. “C₁₋₁₀ alkyl unsubstituted or substituted by at least one group from combination A” has a similar definition only for the total number of carbon atoms.

“C₁₋₁₂ alkoxy unsubstituted or substituted by at least one group from combination A” means that there are no substituents in “C₁₋₁₂ alkoxy” or that there are substitutions by one or more groups from combination A. “alkoxy of C₁₋₁₀ unsubstituted or substituted by at least one group from combination A” has a similar definition, merely differing in the total number of carbon atoms.

“C₃₋₁₂ cycloalkyl unsubstituted or substituted by at least one group from combination A” means the case where “C₃₋₁₂ alkoxy” has no substituent or is substituted by one or more groups from combination A. “C₃₋₁₀ cycloalkyl unsubstituted or substituted by at least one group from combination A” has a similar definition, merely differing in the total number of carbon atoms.

“phenyl unsubstituted or substituted by at least one group from combination A” means the case where “phenyl” has no substituent or is substituted by one or more groups from combination A.

“C₃₋₁₂ cycloalkyl containing at least one hetero atom as a ring-forming atom” means that at least one of the ring-forming atoms of “C₃₋₁₂ cycloalkyl” is a hetero atom other than carbon, and may be exemplified by an oxygen atom, a sulfur atom and a nitrogen atom. “C₃₋₁₀ cycloalkyl containing at least one hetero atom as a ring-constituting atom” has a similar definition to that of “C₃₋₁₂ cycloalkyl” except that the total number of carbon atoms is different from that of “C₃₋₁₂ cycloalkyl”.

“C₂₋₁₂ hydrocarbon containing at least one unsaturated carbon-carbon double bond or unsaturated carbon-carbon triple bond” means that the C₂₋₁₂ hydrocarbon contains at least one unsaturated carbon-carbon double bond or unsaturated carbon-carbon triple bond. “C₂₋₁₀ hydrocarbyl containing at least one unsaturated carbon-carbon double bond or unsaturated carbon-carbon triple bond” has a similar definition, except that the total number of carbon atoms is different.

“C₂₋₁₂ hydrocarbon containing at least one unsaturated carbon-carbon double bond or unsaturated carbon-carbon triple bond which is unsubstituted or substituted by at least one group from combination A” means the case where there is no substituent in “C₂₋₁₂ hydrocarbon radical containing at least one unsaturated carbon-carbon double bond or unsaturated carbon-carbon triple bond” or where there is substitution by one or more groups from combination A. “C₂₋₁₀ hydrocarbon containing at least one unsaturated carbon-carbon double bond or unsaturated carbon-carbon triple bond, unsubstituted or substituted by at least one group from combination A” has a similar definition, merely differing in the total number of carbon atoms.

In the structural formula of the compound provided by the invention, a wavy line represents a bond to a parent nucleus structure.

As described hereinbefore, the first aspect of the present invention provides a compound having a pyrazole structure, or an agrochemically acceptable salt, hydrate and solvate thereof, the compound having a structure represented by formula (I):

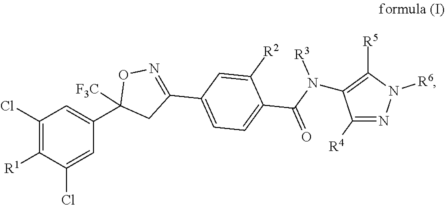

wherein, in the formula (I),
R¹ is selected from H, halogen, at least one halogen substituted C₁₋₆ alkyl, at least one halogen substituted C₁₋₆ alkoxy;
R² is selected from C₁₋₁₂ alkyl, C₁₋₁₂ alkoxy, halogen, at least one halogen-substituted C₁₋₆ alkyl, at least one halogen-substituted C₁₋₆ alkoxy and cyano;
R³ is selected from H, C₁₋₁₂ alkyl, C₁₋₁₂ alkoxy, C₃₋₁₂ cycloalkyl, —CO—R₃₁, —CO—CH₂—R₃₁; R₃₁ is selected from C₁₋₁₂ alkyl, C₃₋₁₂ cycloalkyl and C₁₋₁₂ alkoxy;
R⁴, R⁵ are respectively and independently selected from H, C₁₋₁₂ alkyl, C₁₋₁₂ alkoxy, halogen, at least one halogen-substituted C₁₋₆ alkyl and at least one halogen-substituted C₁₋₆ alkoxy;
R⁶ is selected from the group consisting of H, C₁₋₁₂ alkyl unsubstituted or substituted by at least one group from combination A, C₁₋₁₂ alkoxy unsubstituted or substituted by at least one group from combination A, C₃₋₁₂ cycloalkyl unsubstituted or substituted by at least one group from combination A, phenyl unsubstituted or substituted by at least one group from combination A, C₃₋₁₂ cycloalkyl containing at least one heteroatom as a ring-forming atom, C₂₋₁₂ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂, R₆₁ is selected from the group consisting of C₃₋₁₂ cycloalkyl, C₃₋₁₂ cycloalkyl containing at least one heteroatom as a ring-forming atom, phenyl, R₆₂ is selected from the group consisting of C₁₋₁₂ alkyl, phenyl unsubstituted or substituted by at least one of group A, C₃₋₁₂ cycloalkyl, C₃₋₁₂ cycloalkyl containing at least one heteroatom as a ring-forming atom; R₆₃, R₆₄ are independently selected from C₁₋₁₂ alkyl; each of the heteroatoms is independently at least one selected from N, O, S; each n is independently 1 or 2;
the combination A consists of hydroxyl, halogen, C₁₋₁₂ alkyl, C₁₋₁₂ alkoxy and trimethylsilyl.

Preferably, in formula (I), R¹ is selected from H, halogen, at least one halogen-substituted C₁₋₆ alkyl, at least one halogen-substituted C₁₋₆ alkoxy; more preferably, R¹ is selected from H, F, chloro, bromo.

Preferably, in the formula (I), R² is selected from C₁₋₈ alkyl, C₁₋₈ alkoxy, halogen, at least one halogen-substituted C₁₋₆ alkyl, at least one halogen-substituted C₁₋₆ alkoxy and cyano; more preferably, R² is selected from methyl, ethyl, n-propyl, isopropyl, n-butyl, tert-butyl, cyano.

Preferably, in formula (I), R³ is selected from the group consisting of H, C₁₋₈ alkyl, C₁₋₈ alkoxy, C₃₋₁₀ cycloalkyl, —CO—R₃₁, —CO—CH₂—R₃₁; R₃₁ is selected from C₁₋₈ alkyl, C₃₋₁₀ cycloalkyl and C₁₋₈ alkoxy; more preferably, R³ is selected from the group consisting of H, methyl, ethyl, n-propyl, isopropyl, n-butyl, tert-butyl, methoxy, ethoxy, n-propoxy, isopropoxy, n-butoxy, tert-butoxy, cyclopropyl, cyclobutyl, cyclopentyl, cyclohexyl, —CO—R₃₁, —CO—CH₂—R₃₁; R₃₁ is selected from C₁₋₆ alkyl, C₃₋₆ cycloalkyl and C₁₋₆ alkoxy.

Preferably, in the formula (I), R⁴, R⁵ are independently selected from H, C₁₋₈ alkyl, C₁₋₈ alkoxy, halogen, at least one halogen-substituted C₁₋₆ alkyl and at least one halogen-substituted C₁₋₆ alkoxy; more preferably, R⁴, R⁵ are each independently selected from H, methyl, ethyl, n-propyl, isopropyl, n-butyl, tert-butyl.

Preferably, in formula (I), R⁶ is selected from the group consisting of H, C₁₋₁₀ alkyl unsubstituted or substituted by at least one group from combination A, C₁₋₁₀ alkoxy unsubstituted or substituted by at least one group from combination A, C₃₋₁₀ cycloalkyl unsubstituted or substituted by at least one group from combination A, phenyl unsubstituted or substituted by at least one group from combination A, C₃₋₁₀ cycloalkyl containing at least one heteroatom as a ring-forming atom, C₂₋₁₀ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂; R₆₁ is selected from the group consisting of C₃₋₁₀ cycloalkyl, C₃₋₁₀ cycloalkyl containing at least one heteroatom as a ring-forming atom, phenyl; R₆₂ is selected from the group consisting of C₁₋₁₀ alkyl, phenyl unsubstituted or substituted by at least one of the groups in combination A, C₃₋₁₀ cycloalkyl, C₃₋₁₀ cycloalkyl containing at least one heteroatom as a ring-forming atom; R₆₃, R₆₄ are independently selected from C₁₋₁₀ alkyl; each of the heteroatoms is independently at least one selected from N, O, S; each n is independently 1 or 2; the combination A consists of hydroxyl, halogen, C₁₋₈ alkyl, C₁₋₈ alkoxy and trimethylsilyl; more preferably, R⁶ is selected from the group consisting of H, C₁₋₁₀ alkyl unsubstituted or substituted by at least one group from combination A, C₁₁ alkoxy unsubstituted or substituted by at least one group from combination A, C₃₋₁₀ cycloalkyl unsubstituted or substituted by at least one group from combination A, phenyl unsubstituted or substituted by at least one group from combination A, C₃₋₁₀ cycloalkyl containing at least one heteroatom as a ring-forming atom, C₂₋₁₀ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂; R₆₁ is selected from the group consisting of C₃₋₁₀ cycloalkyl, C₃₋₁₀ cycloalkyl containing at least one heteroatom as a ring-forming atom, phenyl; R₆₂ is selected from the group consisting of C₁₋₁₀ alkyl, phenyl unsubstituted or substituted by at least one of the groups in combination A, C₃₋₁₀ cycloalkyl, C₃₋₁₀ cycloalkyl containing at least one heteroatom as a ring-forming atom; R₆₃, R₆₄ are independently selected from C₁₋₁₀ alkyl; each of the heteroatoms is independently at least one selected from N, O, S; each n is independently 1 or 2; the combination A consists of hydroxyl, halogen, C₁₋₈ alkyl, C₁₋₈ alkoxy and trimethylsilyl.

Preferably, in the formula (I),
R¹ is selected from H, halogen, at least one halogen substituted C₁₋₆ alkyl, at least one halogen substituted C₁₋₆ alkoxy;
R² is selected from C₁₋₈ alkyl, C₁₋₈ alkoxy, halogen, at least one halogen-substituted C₁₋₆ alkyl, at least one halogen-substituted C₁₋₆ alkoxy and cyano;
R³ is selected from H, C₁₋₈ alkyl, C₁₋₈ alkoxy, C₃₋₁₀ cycloalkyl, —CO—R₃₁, —CO—CH₂—R₃₁; R₃₁ is selected from C₁₋₈ alkyl, C₃₋₁₀ cycloalkyl and C₁₋₈ alkoxy;
R⁴, R⁵ are respectively and independently selected from H, C₁₋₈ alkyl, C₁₋₈ alkoxy, halogen, at least one halogen-substituted C₁₋₆ alkyl and at least one halogen-substituted C₁₋₆ alkoxy;
R⁶ is selected from the group consisting of H, C₁₋₁₀ alkyl unsubstituted or substituted by at least one group from combination A, C₁₋₁₀ alkoxy unsubstituted or substituted by at least one group from combination A, C₃₋₁₀ cycloalkyl unsubstituted or substituted by at least one group from combination A, phenyl unsubstituted or substituted by at least one group from combination A, C₃₋₁₀ cycloalkyl containing at least one heteroatom as a ring-forming atom, C₂₋₁₀ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂; R₆₁ is selected from the group consisting of C₃₋₁₀ cycloalkyl, C₃₋₁₀ cycloalkyl containing at least one heteroatom as a ring-forming atom, phenyl; R₆₂ is selected from the group consisting of C₁₋₁₀ alkyl, phenyl unsubstituted or substituted by at least one of the groups in combination A, C₃₋₁₀ cycloalkyl, C₃₋₁₀ cycloalkyl containing at least one heteroatom as a ring-forming atom; R₆₃ and R₆₄ are independently selected from C₁₋₁₀ alkyl; each of the heteroatoms is independently at least one selected from N, O, S; each n is independently 1 or 2;
the combination A consists of hydroxyl, halogen, C₁₋₈ alkyl, C₁₋₈ alkoxy and trimethylsilyl.

More preferably, in formula (I),
R¹ is selected from H, F, chloro, bromo;
R² is selected from methyl, ethyl, n-propyl, isopropyl, n-butyl, tert-butyl, cyano;
R³ is selected from the group consisting of H, methyl, ethyl, n-propyl, isopropyl, n-butyl, tert-butyl, methoxy, ethoxy, n-propoxy, isopropoxy, n-butoxy, tert-butoxy, cyclopropyl, cyclobutyl, cyclopentyl, cyclohexyl, —CO—R₃₁, —CO—CH₂—R₃₁; R₃₁ is selected from C₁₋₆ alkyl, C₃₋₆ cycloalkyl and C₁₋₆ alkoxy;
R⁴, R⁵ are each independently selected from H, methyl, ethyl, n-propyl, isopropyl, n-butyl, tert-butyl;
R⁶ is selected from the group consisting of H, C₁₋₁₀ alkyl unsubstituted or substituted by at least one group from combination A, C₁₋₁₀ alkoxy unsubstituted or substituted by at least one group from combination A, C₃₋₁₀ cycloalkyl unsubstituted or substituted by at least one group from combination A, phenyl unsubstituted or substituted by at least one group from combination A, C₃₋₁₀ cycloalkyl containing at least one heteroatom as a ring-forming atom, C₂₋₁₀ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂; R₆₁ is selected from the group consisting of C₃₋₁₀ cycloalkyl, C₃₋₁₀ cycloalkyl containing at least one heteroatom as a ring-forming atom, phenyl; R₆₂ is selected from the group consisting of C₁₋₁₀ alkyl, phenyl unsubstituted or substituted by at least one of the groups in combination A, C₃₋₁₀ cycloalkyl, C₃₋₁₀ cycloalkyl containing at least one heteroatom as a ring-forming atom; R₆₃, R₆₄ are independently selected from C₁₋₁₀ alkyl; each of the heteroatoms is independently at least one selected from N, O, S; each n is independently 1 or 2; the combination A consists of hydroxyl, halogen, C₁₋₈ alkyl, C₁₋₈ alkoxy and trimethylsilyl.

According to a particularly preferred embodiment, the compound is selected from any one of the following:
Number A1: R¹ is H, R² is CH₃, R³ is H, R⁴ is H, R⁵ is H, R⁶ is —H;
Number A2: R¹ is H, R² is CH₃, R³ is H, R⁴ is H, R⁵ is H, R⁶ is —CH₃;
Number A3: R¹ is H, R² is CH₃, R³ is H, R⁴ is H, R⁵ is H, R⁶ is —CHF₂;
Number A4: R¹ is H, R² is CH₃, R³ is H, R⁴ is H, R⁵ is H, R⁶ is —CF₃;
Number A5: R¹ is H, R² is CH₃, R³ is H, R⁴ is H, R⁵ is H, R⁶ is —CH₂CH₃;
Number A6: R¹ is H, R² is CH₃, R³ is H, R⁴ is H, R⁵ is H, R⁶ is —CH₂CH₂CH₃;
Number A7: R¹ is H, R² is CH₃, R³ is H, R⁴ is H, R⁵ is H, R⁶ is —CH(CH₃)₂;
Number A8: R¹ is H, R² is CH₃, R³ is H, R⁴ is H, R⁵ is H, R⁶ is —CH₂CH₂OH;
Number A9: R¹ is H, R² is CH₃, R³ is H, R⁴ is H, R⁵ is H, R⁶ is —CH₂CF₃;
Number A10: R¹ is H, R² is CH₃, R³ is H, R⁴ is H, R⁵ is H, R⁶ is —CH₂CHF₂;
Number A11: R¹ is H, R² is CH₃, R³ is H, R⁴ is H, R⁵ is H, R⁶ is —CH₂CH₂F;
Number A12: R¹ is H, R² is CH₃, R³ is H, R⁴ is H, R⁵ is H, R⁶ is —CH₂CH₂CF₃;
Number A13: R¹ is H, R² is CH₃, R³ is H, R⁴ is H, R⁵ is H, R⁶ is —CH₂CH₂CH₂F;
Number A14: R¹ is H, R² is CH₃, R³ is H, R⁴ is H, R⁵ is H, R⁶ is —CH₂CH₂OCH₃;
Number A15: R¹ is H, R² is CH₃, R³ is H, R⁴ is H, R⁵ is H, R⁶ is —CH₂CH₂OCH₂CH₃;
Number A16: R¹ is H, R² is CH₃, R³ is H, R⁴ is H, R⁵ is H, R⁶ is —CH₂(CH₂)₂OCH₃;
Number A17: R¹ is H, R² is CH₃, R³ is H, R⁴ is H, R⁵ is H, R⁶ is —CH₂CH₂CH₂CH₃;
Number A18: R¹ is H, R² is CH₃, R³ is H, R⁴ is H, R⁵ is H, R⁶ is —CH(CH₃)CH₂CH₃;
Number A19: R¹ is H, R² is CH₃, R³ is H, R⁴ is H, R⁵ is H, R⁶ is —CH₂(CH₂)₃CH₃;
Number A20: R¹ is H, R² is CH₃, R³ is H, R⁴ is H, R⁵ is H, R⁶ is

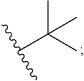 [SMILES: *C(C)(C)C ; ";"]

Number A21: R¹ is H, R² is CH₃, R³ is H, R⁴ is H, R⁵ is H, R⁶ is

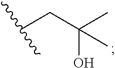 [SMILES: *CC(C)(C)O]

Number A22: R¹ is H, R² is CH₃, R³ is H, R⁴ is H, R¹ is H, R⁶ is

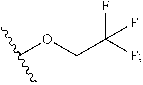 [SMILES: *OCC(F)(F)F]

Number A23: R¹ is H, R² is CH₃, R³ is H, R⁴ is H, R⁵ is H, R⁶ is

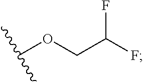 [SMILES: *OCC(F)F]

Number A24: R¹ is H, R² is CH₃, R³ is H, R⁴ is H, R¹ is H, R⁶ is

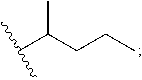 [SMILES: *C(C)CCC]

Number A25: R¹ is H, R² is CH₃, R³ is H, R⁴ is H, R⁵ is H, R⁶ is

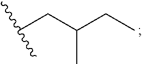 [SMILES: *CC(C)CC]

Number A26: R¹ is H, R² is CH₃, R³ is H, R⁴ is H, R⁵ is H, R⁶ is

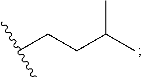 [SMILES: *CCC(C)C]

Number A27: R¹ is H, R² is CH₃, R³ is H, R⁴ is H, R⁵ is H, R⁶ is

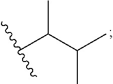 [SMILES: *C(C)C(C)C]

Number A28: R¹ is H, R² is CH₃, R³ is H, R⁴ is H, R⁵ is H, R⁶ is

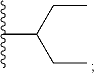 [SMILES: *C(CC)CC]

Number A29: R¹ is H, R² is CH₃, R³ is H, R⁴ is H, R⁵ is H, R⁶ is

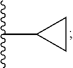 [SMILES: *C1CC1]

Number A30: R¹ is H, R² is CH₃, R³ is H, R⁴ is H, R⁵ is H, R⁶ is

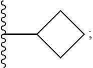 [SMILES: *C1CCC1]

Number A31: R¹ is H, R² is CH₃, R³ is H, R⁴ is H, R⁵ is H, R⁶ is

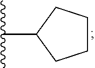 [SMILES: *C1CCCC1]

Number A32: R¹ is H, R² is CH₃, R³ is H, R⁴ is H, R⁵ is H, R⁶ is

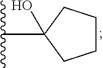 [SMILES: *C1(O)CCCC1]

Number A33: R¹ is H, R² is CH₃, R³ is H, R⁴ is H, R⁵ is H, R⁶ is

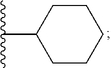 [SMILES: *C1CCCCC1]

Number A34: R¹ is H, R² is CH₃, R³ is H, R⁴ is H, R⁵ is H, R⁶ is

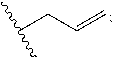 [SMILES: *CC=C]

Number A35: R¹ is H, R² is CH₃, R³ is H, R⁴ is H, R⁵ is H, R⁶ is

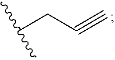 [SMILES: *CC#C]

Number A36: R¹ is H, R² is CH₃, R³ is H, R⁴ is H, R⁵ is H, R⁶ is

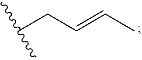 [SMILES: *C/C=C/C]

Number A37: R¹ is H, R² is CH₃, R³ is H, R⁴ is H, R⁵ is H, R⁶ is

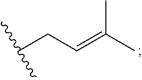 [SMILES: *CC=C(C)C]

Number A38: R¹ is H, R² is CH₃, R³ is H, R⁴ is H, R⁵ is H, R⁶ is

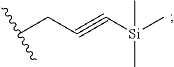 [SMILES: *CC#C[Si](C)(C)C]

Number A39: R¹ is H, R² is CH₃, R³ is H, R⁴ is H, R⁵ is H, R⁶ is

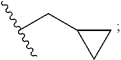 [SMILES: *CC1CC1]

Number A40: R¹ is H, R² is CH₃, R³ is H, R⁴ is H, R⁵ is H, R⁶ is

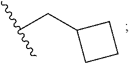 [SMILES: *CC1CCC1]

Number A41: R¹ is H, R² is CH₃, R³ is H, R⁴ is H, R⁵ is H, R⁶ is

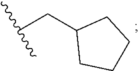 [SMILES: *CC1CCCC1]

Number A42: R¹ is H, R² is CH₃, R³ is H, R⁴ is H, R⁵ is H, R⁶ is

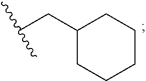 [SMILES: *CC1CCCCC1]

Number A43: R¹ is H, R² is CH₃, R³ is H, R⁴ is H, R⁵ is H, R⁶ is

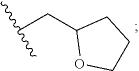 [SMILES: *CC1CCCO1]

Number A44: R¹ is H, R² is CH₃, R³ is H, R⁴ is H, R⁵ is H, R⁶ is

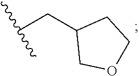 [SMILES: *CC1CCOC1]

Number A45: R¹ is H, R² is CH₃, R³ is H, R⁴ is H, R⁵ is H, R⁶ is

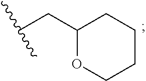 [SMILES: *CC1CCCCO1]

Number A46: R¹ is H, R² is CH₃, R³ is H, R⁴ is H, R⁵ is H, R⁶ is

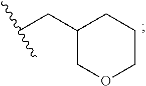 [SMILES: *CC1CCCOC1]

Number A47: R¹ is H, R² is CH₃, R³ is H, R⁴ is H, R⁵ is H, R⁶ is

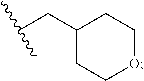 [SMILES: *CC1CCOCC1]

Number A48: R¹ is H, R² is CH₃, R³ is H, R⁴ is H, R⁵ is H, R⁶ is

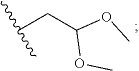 [SMILES: *CC(OC)OC]

Number A49: R¹ is H, R² is CH₃, R³ is H, R⁴ is H, R⁵ is H, R⁶ is

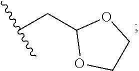 [SMILES: *CC1OCCO1]

Number A50: R¹ is H, R² is CH₃, R³ is H, R⁴ is H, R³ is

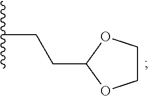 [SMILES: *CCC1OCCO1]

Number A51: R¹ is H, R² is CH₃, R³ is H, R⁴ is H, R⁵ is H, R⁶ is

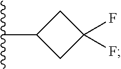 [SMILES: *C1CC(F)(F)C1]

Number A52: R¹ is H, R² is CH₃, R³ is H, R⁴ is H, R⁵ is H, R⁶ is

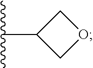 [SMILES: *C1COC1]

Number A53: R¹ is H, R² is CH₃, R³ is H, R⁴ is H, R⁵ is H, R⁶ is

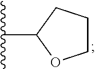 [SMILES: *C1CCCO1]

Number A54: R¹ is H, R² is CH₃, R³ is H, R⁴ is H, R⁵ is H, R⁶ is

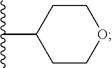 [SMILES: *C1CCOCC1]

Number A55: R¹ is H, R² is CH₃, R³ is H, R⁴ is H, R⁵ is H, R⁶ is

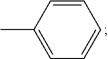

Number A56: R¹ is H, R² is CH₃, R³ is H, R⁴ is H, R⁵ is H, R⁶ is

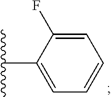 [SMILES: *c1ccccc1F]

Number A57: R¹ is H, R² is CH₃, R³ is H, R⁴ is H, R⁵ is H, R⁶ is

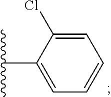 [SMILES: *c1ccccc1Cl]

Number A58: R¹ is H, R² is CH₃, R³ is H, R⁴ is H, R⁵ is H, R⁶ is

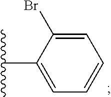 [SMILES: *c1ccccc1Br]

Number A59: R¹ is H, R² is CH₃, R³ is H, R⁴ is H, R⁵ is H, R⁶ is

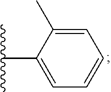 [SMILES: *c1ccccc1C ; ";"]

Number A60: R¹ is H, R² is CH₃, R³ is H, R⁴ is H, R⁵ is H, R⁶ is

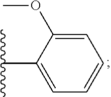 [SMILES: *c1ccccc1OC ; ";"]

Number A61: R¹ is H, R² is CH₃, R³ is H, R⁴ is H, R⁵ is H, R⁶ is

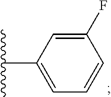 [SMILES: *c1cccc(F)c1]

Number A62: R¹ is H, R² is CH₃, R³ is H, R⁴ is H, R⁵ is H, R⁶ is

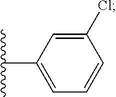 [SMILES: *c1cccc(Cl)c1]

Number A63: R¹ is H, R² is CH₃, R³ is H, R⁴ is H, R⁵ is H, R⁶ is

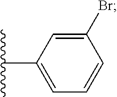 [SMILES: *c1cccc(Br)c1]

Number A64: R¹ is H, R² is CH₃, R³ is H, R⁴ is H, R⁵ is H, R⁶ is

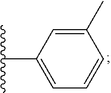 [SMILES: *c1cccc(C)c1 ; ";"]

Number A65: R¹ is H, R² is CH₃, R³ is H, R⁴ is H, R⁵ is H, R⁶ is

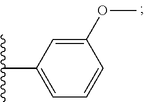 [SMILES: *c1cccc(OC)c1]

Number A66: R¹ is H, R² is CH₃, R³ is H, R⁴ is H, R⁵ is H, R⁶ is

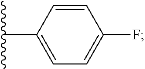 [SMILES: *c1ccc(F)cc1]

Number A67: R¹ is H, R² is CH₃, R³ is H, R⁴ is H, R⁵ is H, R⁶ is

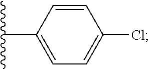 [SMILES: *c1ccc(Cl)cc1]

Number A68: R¹ is H, R² is CH₃, R³ is H, R⁴ is H, R⁵ is H, R⁶ is

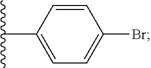 [SMILES: *c1ccc(Br)cc1]

Number A69: R¹ is H, R² is CH₃, R³ is H, R⁴ is H, R⁵ is H, R⁶ is

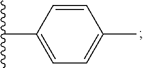 [SMILES: *c1ccc(C)cc1]

Number A70: R¹ is H, R² is CH₃, R³ is H, R⁴ is H, R⁵ is H, R⁶ is

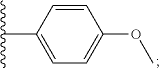 [SMILES: *c1ccc(OC)cc1]

Number A71: R¹ is H, R² is CH₃, R³ is H, R⁴ is H, R⁵ is H, R⁶ is

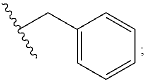 [SMILES: *Cc1ccccc1]

Number A72: R¹ is H, R² is CH₃, R³ is H, R⁴ is H, R⁵ is H, R⁶ is —COCH₃;
Number A73: R¹ is H, R² is CH₃, R³ is H, R⁴ is H, R⁵ is H, R⁶ is —COCH₂CH₃
Number A74: R¹ is H, R² is CH₃, R³ is H, R⁴ is H, R⁵ is H, R⁶ is —COCH₂CH₃)₂;
Number A75: R¹ is H, R² is CH₃, R³ is H, R⁴ is H, R⁵ is H, R⁶ is —COCH(CH₂)₂;
Number A76: R¹ is H, R² is CH₃, R³ is H, R⁴ is H, R⁵ is H, R⁶ is —COC₆H₅;
Number A77: R¹ is H, R² is CH₃, R³ is H, R⁴ is H, R⁵ is H, R⁶ is

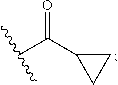 [SMILES: *C(=O)C1CC1]

Number A78: R¹ is H, R² is CH₃, R³ is H, R⁴ is H, R⁵ is H, R⁶ is

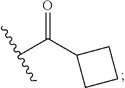 [SMILES: *C(=O)C1CCC1]

Number A79: R¹ is H, R² is CH₃, R³ is H, R⁴ is H, R⁵ is H, R⁶ is

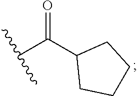 [SMILES: *C(=O)C1CCCC1]

Number A80: R¹ is H, R² is CH₃, R³ is H, R⁴ is H, R⁵ is H, R⁶ is

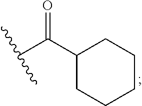 [SMILES: *C(=O)C1CCCCC1]

Number A81: R¹ is H, R² is CH₃, R³ is H, R⁴ is H, R⁵ is H, R⁶ is

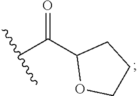 [SMILES: *C(=O)C1CCCO1]

Number A82: R¹ is H, R² is CH₃, R³ is H, R⁴ is H, R⁵ is H, R⁶ is

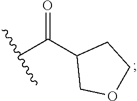 [SMILES: *C(=O)C1CCOC1]

Number A82: R¹ is H, R² is CH₃, R³ is H, R⁴ is H, R⁵ is H, R⁶ is

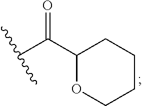 [SMILES: *C(=O)C1CCCCO1]

Number A84: R¹ is H, R² is CH₃, R³ is H, R⁴ is H, R⁵ is H, R⁶ is

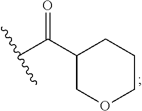 [SMILES: *C(=O)C1CCCOC1]

Number A85: R¹ is H, R² is CH₃, R³ is H, R⁴ is H, R⁵ is H, R⁶ is

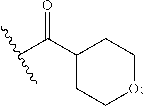 [SMILES: *C(=O)C1CCOCC1]

Number A86: R¹ is H, R² is CH₃, R³ is H, R⁴ is H, R⁵ is H, R⁶ is

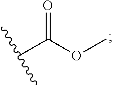 [SMILES: *C(=O)OC]

Number A87: R¹ is H, R² is CH₃, R³ is H, R⁴ is H, R⁵ is H, R⁶ is

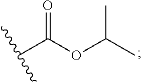 [SMILES: *C(=O)OC(C)C]

Number A88: R¹ is H, R² is CH₃, R³ is H, R⁴ is H, R⁵ is H, R⁶ is

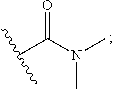 [SMILES: *C(=O)N(C)C]

Number A89: R¹ is H, R² is CH₃, R³ is H, R⁴ is H, R⁵ is H, R⁶ is

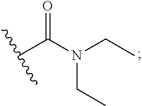 [SMILES: *C(=O)N(CC)CC]

Number A90: R¹ is H, R² is CH₃, R³ is H, R⁴ is H, R⁵ is H, R⁶ is —S(═O)₂CH₃;
Number A91: R¹ is H, R² is CH₃, R³ is H, R⁴ is H, R⁵ is H, R⁶ is —S(═O)₂CH₂CH₃;
Number A92: R¹ is H, R² is CH₃, R³ is H, R⁴ is H, R⁵ is H, R⁶ is —S(═O)₂CH₂CH₂CH₃;
Number A93: R¹ is H, R² is CH₃, R³ is H, R⁴ is H, R⁵ is H, R⁶ is —S(═O)₂CH(CH₃)₂;
Number A94: R¹ is H, R² is CH₃, R³ is H, R⁴ is H, R⁵ is H, R⁶ is —S(═O)₂C₆H₅;
Number A95: R¹ is H, R² is CH₃, R³ is H, R⁴ is H, R⁵ is H, R⁶ is

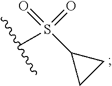 [SMILES: *S(=O)(=O)C1CC1]

Number A96: R¹ is H, R² is CH₃, R³ is H, R⁴ is H, R⁵ is H, R⁶ is

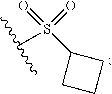 [SMILES: *S(=O)(=O)C1CCC1]

Number A97: R¹ is H, R² is CH₃, R³ is H, R⁴ is H, R⁵ is H, R⁶ is

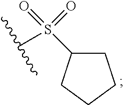 [SMILES: *S(=O)(=O)C1CCCC1]

Number A98: R¹ is H, R² is CH₃, R³ is H, R⁴ is H, R⁵ is H, R⁶ is

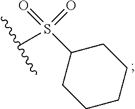 [SMILES: *S(=O)(=O)C1CCCCC1]

Number A99: R¹ is H, R² is CH₃, R³ is H, R⁴ is H, R⁵ is H, R⁶ is

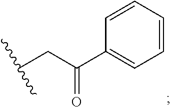 [SMILES: *CC(=O)c1ccccc1]

Number A100: R¹ is H, R² is CH₃, R³ is H, R⁴ is H, R⁵ is H, R⁶ is

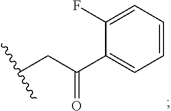 [SMILES: *CC(=O)c1ccccc1F]

Number A101: R¹ is H, R² is CH₃, R³ is H, R⁴ is H, R⁵ is H, R⁶ is

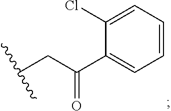 [SMILES: *CC(=O)c1ccccc1Cl]

Number A102: R¹ is H, R² is CH₃, R³ is H, R⁴ is H, R⁵ is H, R⁶ is

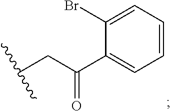 [SMILES: *CC(=O)c1ccccc1Br]

Number A103: R¹ is H, R² is CH₃, R³ is H, R⁴ is H, R⁵ is H, R⁶ is

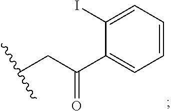 [SMILES: *CC(=O)c1ccccc1I]

Number A104: R¹ is H, R² is CH₃, R³ is H, R⁴ is H, R⁵ is H, R⁶ is

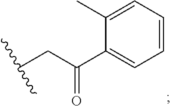 [SMILES: *CC(=O)c1ccccc1C]

Number A105: R¹ is H, R² is CH₃, R³ is H, R⁴ is H, R⁵ is H, R⁶ is

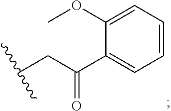 [SMILES: *CC(=O)c1ccccc1OC]

Number A106: R¹ is H, R² is CH₃, R³ is H, R⁴ is H, R⁵ is H, R⁶ is

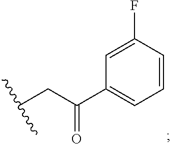 [SMILES: *CC(=O)c1cccc(F)c1]

Number A107: R¹ is H, R² is CH₃, R³ is H, R⁴ is H, R⁵ is H, R⁶ is

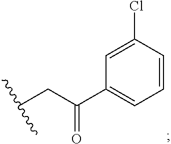 [SMILES: *CC(=O)c1cccc(Cl)c1]

Number A108: R¹ is H, R² is CH₃, R³ is H, R⁴ is H, R⁵ is H, R⁶ is

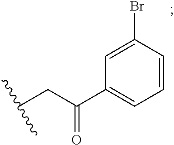 [SMILES: *CC(=O)c1cccc(Br)c1]

Number A109: R¹ is H, R² is CH₃, R³ is H, R⁴ is H, R⁵ is H, R⁶ is

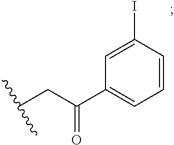 [SMILES: *CC(=O)c1cccc(I)c1]

Number A110: R¹ is H, R² is CH₃, R³ is H, R⁴ is H, R⁵ is H, R⁶ is

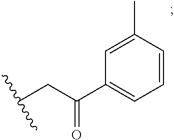 [SMILES: *CC(=O)c1cccc(C)c1]

Number A11: R¹ is H, R² is CH₃, R³ is H, R⁴ is H, R⁵ is H, R⁶ is

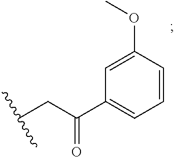 [SMILES: *CC(=O)c1cccc(OC)c1]

Number A112: R¹ is H, R² is CH₃, R³ is H, R⁴ is H, R⁵ is H, R⁶ is

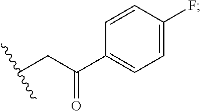 [SMILES: *CC(=O)c1ccc(F)cc1]

Number A113: R¹ is H, R² is CH₃, R³ is H, R⁴ is H, R⁵ is H, R⁶ is

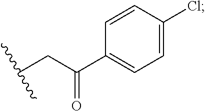 [SMILES: *CC(=O)c1ccc(Cl)cc1]

Number A114: R¹ is H, R² is CH₃, R³ is H, R⁴ is H, R⁵ is H, R⁶ is

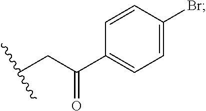 [SMILES: *CC(=O)c1ccc(Br)cc1]

Number A115: R¹ is H, R² is CH₃, R³ is H, R⁴ is H, R⁵ is H, R⁶ is

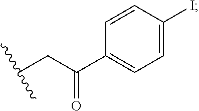 [SMILES: *CC(=O)c1ccc(I)cc1]

Number A116: R¹ is H, R² is CH₃, R³ is H, R⁴ is H, R⁵ is H, R⁶ is

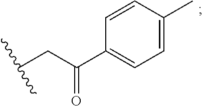 [SMILES: *CC(=O)c1ccc(C)cc1]

Number A117: R¹ is H, R² is CH₃, R³ is H, R⁴ is H, R⁵ is CH₃, R⁶ is

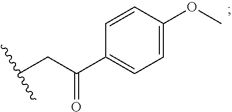 [SMILES: *CC(=O)c1ccc(OC)cc1]

Number A118: R¹ is H, R² is CH₃, R³ is H, R⁴ is H, R⁵ is CH₃, R⁶ is —H;
Number A119: R¹ is H, R² is CH₃, R³ is H, R⁴ is H, R⁵ is CH₃, R⁶ is —CH₃;
Number A120: R¹ is H, R² is CH₃, R³ is H, R⁴ is H, R⁵ is CH₃, R⁶ is —CH₂CH₃;
Number A121: R¹ is H, R² is CH₃, R³ is H, R⁴ is H, R⁵ is CH₃, R⁶ is —CH₂CH₂CH₃;
Number A122: R¹ is H, R² is CH₃, R³ is H, R⁴ is H, R⁵ is CH₃, R⁶ is —CH(CH₃)₂;
Number A123: R¹ is H, R² is CH₃, R³ is H, R⁴ is H, R⁵ is CH₃, R⁶ is

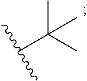 [SMILES: *C(C)(C)C]

Number A124: R¹ is H, R² is CH₃, R³ is H, R⁴ is H, R⁵ is CH₃, R⁶ is

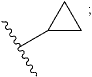 [SMILES: *C1CC1]

Number A125: R¹ is H, R² is CH₃, R³ is H, R⁴ is H, R⁵ is CH₃, R⁶ is

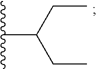 [SMILES: *C(CC)CC]

Number A126: R¹ is H, R² is CH₃, R³ is H, R⁴ is H, R⁵ is CH₃, R⁶ is

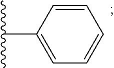 [SMILES: *c1ccccc1]

Number A127: R¹ is H, R² is CH₃, R³ is H, R⁴ is H, R⁵ is CH₃, R⁶ is

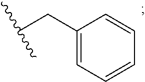 [SMILES: *Cc1ccccc1]

Number A128: R¹ is H, R² is CH₃, R³ is H, R⁴ is H, R⁵ is CH₃, R⁶ is —COCH₃;
Number A129: R¹ is H, R² is CH₃, R³ is H, R⁴ is H, R⁵ is CH₃, R⁶ is —COCH₂CH₃;
Number A130: R¹ is H, R² is CH₃, R³ is H, R⁴ is H, R⁵ is CH₃, R⁶ is —COCH₂CH₂CH₃;
Number A131: R¹ is H, R² is CH₃, R³ is H, R⁴ is H, R⁵ is CH₃, R⁶ is —COCH(CH₃)₂;
Number A132: R¹ is H, R² is CH₃, R³ is H, R⁴ is H, R⁵ is CH₃, R⁶ is —COC₆H₅;
Number A133: R¹ is H, R² is CH₃, R³ is H, R⁴ is H, R⁵ is CH₃, R⁶ is

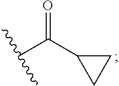 [SMILES: *C(=O)C1CC1]

Number A134: R¹ is H, R² is CH₃, R³ is H, R⁴ is H, R⁵ is CH₃, R⁶ is

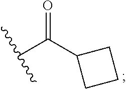 [SMILES: *C(=O)C1CCC1]

Number A135: R¹ is H, R² is CH₃, R³ is H, R⁴ is H, R⁵ is CH₃, R⁶ is

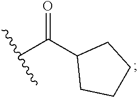 [SMILES: *C(=O)C1CCCC1]

Number A136: R¹ is H, R² is CH₃, R³ is H, R⁴ is H, R⁵ is CH₃, R⁶ is

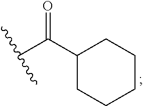 [SMILES: *C(=O)C1CCCCC1]

Number A137: R¹ is H, R² is CH₃, R³ is H, R⁴ is H, R⁵ is CH₃, R⁶ is

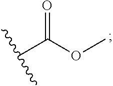 [SMILES: *C(=O)OC]

Number A138: R¹ is H, R² is CH₃, R³ is H, R⁴ is H, R⁵ is CH₃, R⁶ is

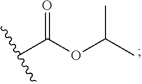 [SMILES: *C(=O)OC(C)C]

Number A139: R¹ is H, R² is CH₃, R³ is H, R⁴ is H, R⁵ is CH₃, R⁶ is

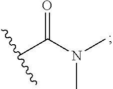 [SMILES: *C(=O)N(C)C]

Number A140: R¹ is H, R² is CH₃, R³ is H, R⁴ is H, R⁵ is CH₃, R⁶ is

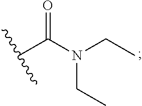 [SMILES: *C(=O)N(CC)CC]

Number A141: R¹ is H, R² is CH₃, R³ is H, R⁴ is H, R⁵ is CH₃, R⁶ is —S(═O)₂CH₂CH₃;
Number A142: R¹ is H, R² is CH₃, R³ is H, R⁴ is H, R⁵ is CH₃, R⁶ is —S(═O)₂CH₂CH₃CH₃;
Number A143: R¹ is H, R² is CH₃, R³ is H, R⁴ is H, R⁵ is CH₃, R⁶ is —S(═O)₂CH₂CH₂C₃;
Number A144: R¹ is H, R² is CH₃, R³ is H, R⁴ is H, R⁵ is CH₃, R⁶ is —S(═O)₂CH₂(CH₃)₂;
Number A145: R¹ is H, R² is CH₃, R³ is H, R⁴ is H, R⁵ is CH₃, R⁶ is —S(═O)₂C₆H₅;
Number A146: R¹ is H, R² is CH₃, R³ is H, R⁴ is H, R⁵ is CH₃, R⁶ is

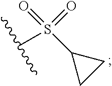 [SMILES: *S(=O)(=O)C1CC1]

Number A147: R¹ is H, R² is CH₃, R³ is H, R⁴ is H, R⁵ is CH₃, R⁶ is

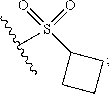 [SMILES: *S(=O)(=O)C1CCC1]

Number A148: R¹ is H, R² is CH₃, R³ is H, R⁴ is H, R⁵ is CH₃, R⁶ is

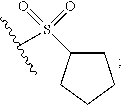 [SMILES: *S(=O)(=O)C1CCCC1]

Number A149: R¹ is H, R² is CH₃, R³ is H, R⁴ is H, R⁵ is CH₃, R⁶ is

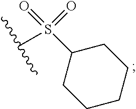 [SMILES: *S(=O)(=O)C1CCCCC1]

Number A150: R¹ is H, R² is CH₃, R³ is H, R⁴ is H, R⁵ is CH₃, R⁶ is —H;
Number A151: R¹ is H, R² is CH₃, R³ is H, R⁴ is H, R⁵ is CH₃, R⁶ is —CH₃;
Number A152: R¹ is H, R² is CH₃, R³ is H, R⁴ is CH₃, R⁵ is CH₃, R⁶ is —CH₂CH₃;
Number A153: R¹ is H, R² is CH₃, R³ is H, R⁴ is CH₃, R⁵ is CH₃, R⁶ is —CH₂CH₂CH₃;
Number A154: R¹ is H, R² is CH₃, R³ is H, R⁴ is CH₃, R⁵ is CH₃, R⁶ is —CH(CH₃)₂;
Number A155: R¹ is H, R² is CH₃, R³ is H, R⁴ is CH₃, R⁵ is CH₃, R⁶ is

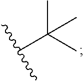 [SMILES: *C(C)(C)C ; ";"]

Number A156: R¹ is H, R² is CH₃, R³ is H, R⁴ is CH₃, R⁵ is CH₃, R⁶ is

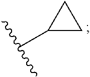 [SMILES: *C1CC1]

Number A157: R¹ is H, R² is CH₃, R³ is H, R⁴ is CH₃, R⁵ is CH₃, R⁶ is

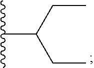 [SMILES: *C(CC)CC]

Number A158: R¹ is H, R² is CH₃, R³ is H, R⁴ is CH₃, R⁵ is CH₃, R⁶ is

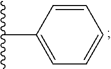 [SMILES: *c1ccccc1]

Number A159: R¹ is H, R² is CH₃, R³ is H, R⁴ is CH₃, R⁵ is CH₃, R⁶ is

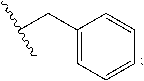 [SMILES: *Cc1ccccc1]

Number A160: R¹ is H, R² is CH₃, R³ is H, R⁴ is CH₃, R⁵ is CH₃, R⁶ is —COCH₃;
Number A161: R¹ is H, R² is CH₃, R³ is H, R⁴ is CH₃, R⁵ is CH₃, R⁶ is —COCH₂CH₃;
Number A162: R¹ is H, R² is CH₃, R³ is H, R⁴ is CH₃, R⁵ is CH₃, R⁶ is —COCH₂CH₂CH₃;
Number A163: R¹ is H, R² is CH₃, R³ is H, R⁴ is CH₃, R⁵ is CH₃, R⁶ is —COCH(CH₃)₂;
Number A164: R¹ is H, R² is CH₃, R³ is H, R⁴ is CH₃, R⁵ is CH₃, R⁶ is —COC₆H₅;
Number A165: R¹ is H, R² is CH₃, R³ is H, R⁴ is CH₃, R⁵ is CH₃, R⁶ is

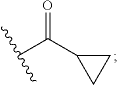 [SMILES: *C(=O)C1CC1]

Number A166: R¹ is H, R² is CH₃, R³ is H, R⁴ is CH₃, R⁵ is CH₃, R⁶ is

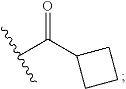 [SMILES: *C(=O)C1CCC1]

Number A167: R¹ is H, R² is CH₃, R³ is H, R⁴ is CH₃, R⁵ is CH₃, R⁶ is

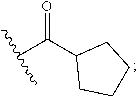 [SMILES: *C(=O)C1CCCC1]

Number A168: R¹ is H, R² is CH₃, R³ is H, R⁴ is CH₃, R⁵ is CH₃, R⁶ is

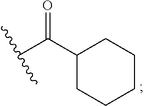 [SMILES: *C(=O)C1CCCCC1]

Number A169: R¹ is H, R² is CH₃, R³ is H, R⁴ is CH₃, R⁵ is CH₃, R⁶ is

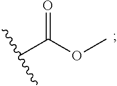 [SMILES: *C(=O)OC]

Number A170: R¹ is H, R² is CH₃, R³ is H, R⁴ is CH₃, R⁵ is CH₃, R⁶ is

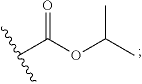 [SMILES: *C(=O)OC(C)C]

Number A171: R¹ is H, R² is CH₃, R³ is H, R⁴ is CH₃, R⁵ is CH₃, R⁶ is

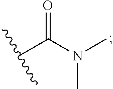 [SMILES: *C(=O)N(C)C]

Number A172: R¹ is H, R² is CH₃, R³ is H, R⁴ is CH₃, R⁵ is CH₃, R⁶ is

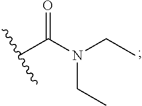 [SMILES: *C(=O)N(CC)CC]

Number A173: R¹ is H, R² is CH₃, R³ is H, R⁴ is CH₃, R⁵ is CH₃, R⁶ is —S(═O)₂CH₃;
Number A174: R¹ is H, R² is CH₃, R³ is H, R⁴ is CH₃, R⁵ is CH₃, R⁶ is —S(═O)₂CH₂CH₃;
Number A175: R¹ is H, R² is CH₃, R³ is H, R⁴ is CH₃, R⁵ is CH₃, R⁶ is —S(═O)₂CH₂CH₂CH₃;
Number A176: R¹ is H, R² is CH₃, R³ is H, R⁴ is CH₃, R⁵ is CH₃, R⁶ is —S(═O)₂CH(CH₃)₂;
Number A177: R¹ is H, R² is CH₃, R³ is H, R⁴ is CH₃, R⁵ is CH₃, R⁶ is —S(═O)₂C₆H₅;
Number A178: R¹ is H, R² is CH₃, R³ is H, R⁴ is CH₃, R⁵ is CH₃, R⁶ is

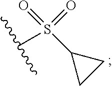 [SMILES: *S(=O)(=O)C1CC1]

Number A179: R¹ is H, R² is CH₃, R³ is H, R⁴ is CH₃, R⁵ is CH₃, R⁶ is

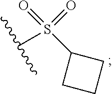 [SMILES: *S(=O)(=O)C1CCC1]

Number A180: R¹ is H, R² is CH₃, R³ is H, R⁴ is CH₃, R⁵ is CH₃, R⁶ is

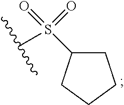 [SMILES: *S(=O)(=O)C1CCCC1]

Number A181: R¹ is H, R² is CH₃, R³ is H, R⁴ is CH₃, R⁵ is CH₃, R⁶ is

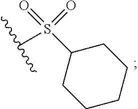 [SMILES: *S(=O)(=O)C1CCCCC1]

Number A182: R¹ is H, R² is CH₃, R³ is CH₃, R⁴ is H, R⁵ is H, R⁶ is

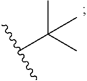 [SMILES: *C(C)(C)C]

Number A183: R⁶ is H, R² is CH₃, R³ is OCH₃, R⁴ is H, R^{C} is H, R⁶ is

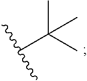 [SMILES: *C(C)(C)C ; ";"]

Number A184: R¹ is H, R² is CH₃, R³ is

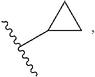 [SMILES: *C1CC1]

R⁴ is H, R⁵ is H, R⁶ is

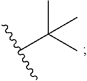 [SMILES: *C(C)(C)C ; ";"]

Number A185: R¹ is H, R² is CH₃, R³ is COCH₃, R⁴ is H, R⁵ is H, R⁶ is

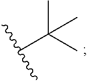 [SMILES: *C(C)(C)C ; ";"]

Number A186: R¹ is H, R² is CH₃, R³ is

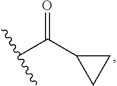 [SMILES: *C(=O)C1CC1]

R⁴ is H, R⁵ is H, R⁶ is

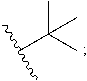 [SMILES: *C(C)(C)C ; ";"]

Number A187: R¹ is H, R² is CH₃, R³ is

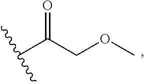 [SMILES: *C(=O)COC]

R⁴ is H, R⁵ is H, R⁶ is

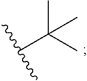 [SMILES: *C(C)(C)C ; ";"]

Number A188: R¹ is H, R² is CH₃, R³ is

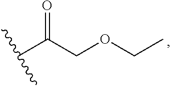 [SMILES: *C(=O)COCC]

R⁴ is H, R⁵ is H, R⁶ is

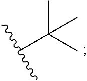 [SMILES: *C(C)(C)C ; ";"]

Number A189: R¹ is F, R² is CH₃, R³ is H, R⁴ is H, R⁵ is H, R⁶ is

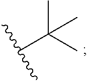 [SMILES: *C(C)(C)C ; ";"]

Number A190: R¹ is F, R² is CH₃, R³ is H, R⁴ is H, R⁵ is H, R⁶ is —H;
Number A191: R¹ is F, R² is CH₃, R³ is H, R⁴ is H, R⁵ is H, R⁶ is —CH₃;
Number A192: R¹ is F, R² is CH₃, R³ is H, R⁴ is H, R⁵ is H, R⁶ is —CH₂CH₃;
Number A193: R¹ is F, R² is CH₃, R³ is H, R⁴ is H, R⁵ is H, R⁶ is —CH₂CH₂CH₃;
Number A194: R¹ is F, R² is CH₃, R³ is H, R⁴ is H, R⁵ is H, R⁶ is —CH(CH₃)₂;
Number A195: R¹ is F, R² is CH₃, R³ is H, R⁴ is H, R⁵ is H, R⁶ is

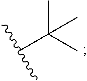 [SMILES: *C(C)(C)C ; ";"]

Number A196: R¹ is F, R² is CH₃, R³ is H, R⁴ is H, R⁵ is H, R⁶ is

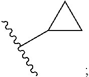 [SMILES: *C1CC1 ; ";"]

Number A197: R¹ is F, R² is CH₃, R³ is H, R⁴ is H, R⁵ is H, R⁶ is

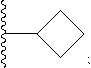 [SMILES: *C1CCC1]

Number A198: R¹ is F, R² is CH₃, R³ is H, R⁴ is H, R⁵ is H, R⁶ is

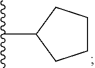 [SMILES: *C1CCCC1]

Number A199: R¹ is F, R² is CH₃, R³ is H, R⁴ is H, R⁵ is H, R⁶ is

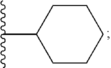 [SMILES: *C1CCCCC1]

Number A200: R¹ is F, R² is CH₃, R³ is H, R⁴ is H, R⁵ is H, R⁶ is

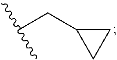 [SMILES: *CC1CC1]

Number A201: R¹ is F, R² is CH₃, R³ is H, R⁴ is H, R⁵ is H, R⁶ is

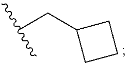 [SMILES: *CC1CCC1]

Number A202: R¹ is F, R² is CH₃, R³ is H, R⁴ is H, R⁵ is H, R⁶ is

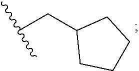 [SMILES: *CC1CCCC1]

Number A203: R¹ is F, R² is CH₃, R³ is H, R⁴ is H, R⁵ is H, R⁶ is

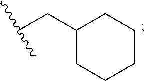 [SMILES: *CC1CCCCC1]

Number A204: R¹ is F, R² is CH₃, R³ is H, R⁴ is H, R⁵ is H, R⁶ is

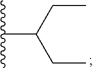 [SMILES: *C(CC)CC]

Number A205: R¹ is F, R² is CH₃, R³ is H, R⁴ is H, R⁵ is H, R⁶ is

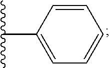 [SMILES: *c1ccccc1]

Number A206: R¹ is F, R² is CH₃, R³ is H, R⁴ is H, R⁵ is H, R⁶ is

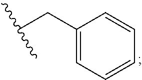 [SMILES: *Cc1ccccc1]

Number A207: R¹ is F, R² is CH₃, R³ is H, R⁴ is H, R⁵ is H, R⁶ is —COCH₃
Number A208: R¹ is F, R² is CH₃, R³ is H, R⁴ is H, R⁵ is H, R⁶ is —COCH₂CH₃;
Number A209: R¹ is F, R² is CH₃, R³ is H, R⁴ is H, R⁵ is H, R⁶ is —COCH₂CH₂CH₃;
Number A210: R¹ is F, R² is CH₃, R³ is H, R⁴ is H, R⁵ is H, R⁶ is —COCH(CH₃)₂;
Number A211: R¹ is F, R² is CH₃, R³ is H, R⁴ is H, R⁵ is H, R⁶ is —COC₆H₅;
Number A212: R¹ is F, R² is CH₃, R³ is H, R⁴ is H, R⁵ is H, R⁶ is

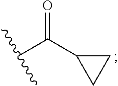 [SMILES: *C(=O)C1CC1]

Number A213: R¹ is F, R² is CH₃, R³ is H, R⁴ is H, R⁵ is H, R⁶ is

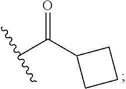 [SMILES: *C(=O)C1CCC1]

Number A214: R¹ is F, R² is CH₃, R³ is H, R⁴ is H, R⁵ is H, R⁶ is

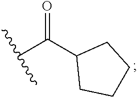 [SMILES: *C(=O)C1CCCC1]

Number A215: R¹ is F, R² is CH₃, R³ is H, R⁴ is H, R⁵ is H, R⁶ is

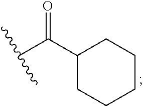 [SMILES: *C(=O)C1CCCCC1]

Number A216: R¹ is F, R² is CH₃, R³ is H, R⁴ is H, R⁵ is H, R⁶ is

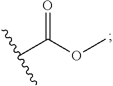 [SMILES: *C(=O)OC]

Number A217: R¹ is F, R² is CH₃, R³ is H, R⁴ is H, R⁵ is H, R⁶ is

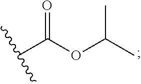 [SMILES: *C(=O)OC(C)C]

Number A218: R¹ is F, R² is CH₃, R³ is H, R⁴ is H, R⁵ is H, R⁶ is

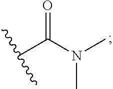 [SMILES: *C(=O)N(C)C]

Number A219: R¹ is F, R² is CH₃, R³ is H, R⁴ is H, R⁵ is H, R⁶ is

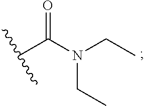 [SMILES: *C(=O)N(CC)CC]

Number A220: R¹ is F, R² is CH₃, R³ is H, R⁴ is H, R⁵ is H, R⁶ is —S(═O)₂CH₃;
Number A221: R¹ is F, R² is CH₃, R³ is H, R⁴ is H, R⁵ is H, R⁶ is —S(═O)₂CH₂CH₃;
Number A222: R¹ is F, R² is CH₃, R³ is H, R⁴ is H, R⁵ is H, R⁶ is —S(═O)₂CH₂CH₂CH₃;
Number A223: R¹ is F, R² is CH₃, R³ is H, R⁴ is H, R⁵ is H, R⁶ is —S(═O)₂CH(CH₃)₂;
Number A224: R¹ is F, R² is CH₃, R³ is H, R⁴ is H, R⁵ is H, R⁶ is —S(═O)₂C₆H₅;
Number A225: R¹ is F, R² is CH₃, R³ is H, R⁴ is H, R⁵ is H, R⁶ is

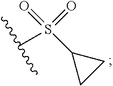 [SMILES: *S(=O)(=O)C1CC1]

Number A226: R¹ is F, R² is CH₃, R³ is H, R⁴ is H, R⁵ is H, R⁶ is

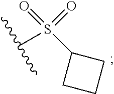 [SMILES: *S(=O)(=O)C1CCC1]

Number A227: R¹ is F, R² is CH₃, R³ is H, R⁴ is H, R⁵ is H, R⁶ is

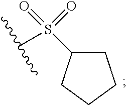 [SMILES: *S(=O)(=O)C1CCCC1]

Number A228: R¹ is F, R² is CH₃, R³ is H, R⁴ is H, R⁵ is H, R⁶ is

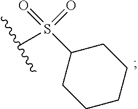 [SMILES: *S(=O)(=O)C1CCCCC1]

Number A229: R¹ is F, R² is CH₃, R³ is H, R⁴ is H, R⁵ is CH₃, R⁶ is —H;
Number A230: R¹ is F, R² is CH₃, R³ is H, R⁴ is H, R⁵ is CH₃, R⁶ is —CH₃;
Number A231: R¹ is F, R² is CH₃, R³ is H, R⁴ is H, R⁵ is CH₃, R⁶ is —CH₂CH₃;
Number A232: R¹ is F, R² is CH₃, R³ is H, R⁴ is H, R⁵ is CH₃, R⁶ is —CH₂CH₂CH₃;
Number A233: R¹ is F, R² is CH₃, R³ is H, R⁴ is H, R⁵ is CH₃, R⁶ is —CH(CH₃)₂;
Number A234: R¹ is F, R² is CH₃, R³ is H, R⁴ is H, R⁵ is CH₃, R⁶ is

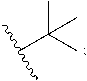 [SMILES: *C(C)(C)C ; ";"]

Number A235: R¹ is F, R² is CH₃, R³ is H, R⁴ is H, R⁵ is CH₃, R⁶ is

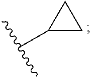 [SMILES: *C1CC1]

Number A236: R¹ is F, R² is CH₃, R³ is H, R⁴ is H, R⁵ is CH₃, R⁶ is

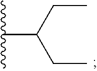 [SMILES: *C(CC)CC]

Number A237: R¹ is F, R² is CH₃, R³ is H, R⁴ is H, R⁵ is CH₃, R⁶ is

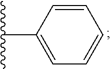 [SMILES: *c1ccccc1]

Number A238: R¹ is F, R² is CH₃, R³ is H, R⁴ is H, R⁵ is CH₃, R⁶ is

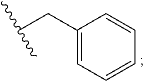 [SMILES: *Cc1ccccc1]

Number A239: R¹ is F, R² is CH₃, R³ is H, R⁴ is H, R⁵ is CH₃, R⁶ is —COCH₃;
Number A240: R¹ is F, R² is CH₃, R³ is H, R⁴ is H, R⁵ is CH₃, R⁶ is —COCH₂CH₃;
Number A241: R¹ is F, R² is CH₃, R³ is H, R⁴ is H, R⁵ is CH₃, R⁶ is —COCH₂CH₂CH₃;
Number A242: R¹ is F, R² is CH₃, R³ is H, R⁴ is H, R⁵ is CH₃, R⁶ is —COCH(CH₃)₂;
Number A243: R¹ is F, R² is CH₃, R³ is H, R⁴ is H, R⁵ is CH₃, R⁶ is —COC₆H₅;
Number A244: R¹ is F, R² is CH₃, R³ is H, R⁴ is H, R⁵ is CH₃, R⁶ is

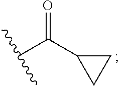 [SMILES: *C(=O)C1CC1]

Number A245: R¹ is F, R² is CH₃, R³ is H, R⁴ is H, R⁵ is CH₃, R⁶ is

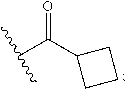 [SMILES: *C(=O)C1CCC1]

Number A246: R¹ is F, R² is CH₃, R³ is H, R⁴ is H, R⁵ is CH₃, R⁶ is

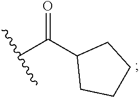 [SMILES: *C(=O)C1CCCC1]

Number A247: R¹ is F, R² is CH₃, R³ is H, R⁴ is H, R⁵ is CH₃, R⁶ is

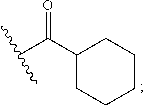 [SMILES: *C(=O)C1CCCCC1]

Number A248: R¹ is F, R² is CH₃, R³ is H, R⁴ is H, R⁵ is CH₃, R⁶ is

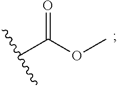 [SMILES: *C(=O)OC]

Number A249: R¹ is F, R² is CH₃, R³ is H, R⁴ is H, R⁵ is CH₃, R⁶ is

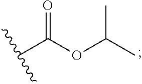 [SMILES: *C(=O)OC(C)C]

Number A250: R¹ is F, R² is CH₃, R³ is H, R⁴ is H, R⁵ is CH₃, R⁶ is

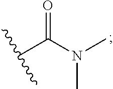 [SMILES: *C(=O)N(C)C]

Number A251: R¹ is F, R² is CH₃, R³ is H, R⁴ is H, R⁵ is CH₃, R⁶ is

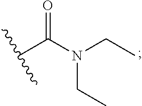 [SMILES: *C(=O)N(CC)CC]

Number A252: R¹ is F, R² is CH₃, R³ is H, R⁴ is H, R⁵ is CH₃, R⁶ is —S(═O)₂CH₃;
Number A253: R¹ is F, R² is CH₃, R³ is H, R⁴ is H, R⁵ is CH₃, R⁶ is —S(═O)CH₂CH₃;
Number A254: R¹ is F, R² is CH₃, R³ is H, R⁴ is H, R⁵ is CH₃, R⁶ is —S(═O)₂CH₂CH₂CH₃;
Number A255: R¹ is F, R² is CH₃, R³ is H, R⁴ is H, R⁵ is CH₃, R⁶ is —S(═O)₂CH(CH₃)₂;
Number A256: R¹ is F, R² is CH₃, R³ is H, R⁴ is H, R⁵ is CH₃, R⁶ is —S(═O)₂C₆H₅;
Number A257: R¹ is F, R² is CH₃, R³ is H, R⁴ is H, R⁵ is CH₃, R⁶ is

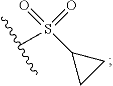 [SMILES: *S(=O)(=O)C1CC1]

Number A258: R¹ is F, R² is CH₃, R³ is H, R⁴ is H, R⁵ is CH₃, R⁶ is

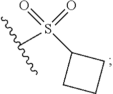 [SMILES: *S(=O)(=O)C1CCC1]

Number A259: R¹ is F, R² is CH₃, R³ is H, R⁴ is H, R⁵ is CH₃, R⁶ is

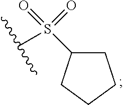 [SMILES: *S(=O)(=O)C1CCCC1]

Number A260: R¹ is F, R² is CH₃, R³ is H, R⁴ is H, R⁵ is CH₃, R⁶ is

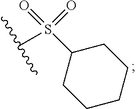 [SMILES: *S(=O)(=O)C1CCCCC1]

Number A261: R¹ is F, R² is CH₃, R³ is H, R⁴ is CH₃, R⁵ is CH₃, R⁶ is —H;
Number A262: R¹ is F, R² is CH₃, R³ is H, R⁴ is CH₃, R⁵ is CH₃, R⁶ is —CH₃
Number A263: R¹ is F, R² is CH₃, R³ is H, R⁴ is CH₃, R⁵ is CH₃, R⁶ is —CH₂CH₃;
Number A264: R¹ is F, R² is CH₃, R³ is H, R⁴ is CH₃, R⁵ is CH₃, R⁶ is —CH₂CH₂CH₃;
Number A265: R¹ is F, R² is CH₃, R³ is H, R⁴ is CH₃, R⁵ is CH₃, R⁶ is —CH(CH₃)₂;
Number A266: R¹ is F, R² is CH₃, R³ is H, R⁴ is CH₃, R⁵ is CH₃, R⁶ is

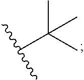 [SMILES: *C(C)(C)C]

Number A267: R¹ is F, R² is CH₃, R³ is H, R⁴ is CH₃, R⁵ is CH₃, R⁶ is

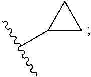 [SMILES: *C1CC1]

Number A268: R¹ is F, R² is CH₃, R³ is H, R⁴ is CH₃, R⁵ is CH₃, R⁶ is

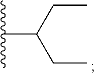 [SMILES: *C(CC)CC]

Number A269: R¹ is F, R² is CH₃, R³ is H, R⁴ is CH₃, R⁵ is CH₃, R⁶ is

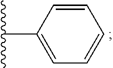 [SMILES: *c1ccccc1]

Number A270: R¹ is F, R² is CH₃, R³ is H, R⁴ is CH₃, R⁵ is CH₃, R⁶ is

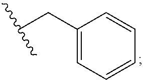 [SMILES: *Cc1ccccc1]

Number A271: R¹ is F, R² is CH₃, R³ is H, R⁴ is CH₃, R⁵ is CH₃, R⁶ is —COCH₃;
Number A272: R¹ is F, R² is CH₃, R³ is H, R⁴ is CH₃, R⁵ is CH₃, R⁶ is —COCH₂C₃;
Number A272: R¹ is F, R² is CH₃, R³ is H, R⁴ is CH₃, R⁵ is CH₃, R⁶ is —COCH₂CH₃;
Number A273: R¹ is F, R² is CH₃, R³ is H, R⁴ is CH₃, R⁵ is CH₃, R⁶ is —COCH₂CH₂CH₃;
Number A274: R¹ is F, R² is CH₃, R³ is H, R⁴ is CH₃, R⁵ is CH₃, R⁶ is —COCH(CH₃)₂;
Number A275: R¹ is F, R² is CH₃, R³ is H, R⁴ is CH₃, R⁵ is CH₃, R⁶ is —COC₆H₃;
Number A276: R¹ is F, R² is CH₃, R³ is H, R⁴ is CH₃, R⁵ is CH₃, R⁶ is

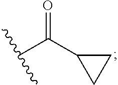 [SMILES: *C(=O)C1CC1]

Number A277: R¹ is F, R² is CH₃, R³ is H, R⁴ is CH₃, R⁵ is CH₃, R⁶ is

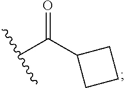 [SMILES: *C(=O)C1CCC1]

Number A278: R¹ is F, R² is CH₃, R³ is H, R⁴ is CH₃, R⁵ is CH₃, R⁶ is

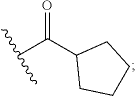 [SMILES: *C(=O)C1CCCC1]

Number A279: R¹ is F, R² is CH₃, R³ is H, R⁴ is CH₃, R⁵ is CH₃, R⁶ is

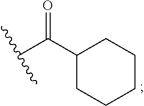 [SMILES: *C(=O)C1CCCCC1]

Number A280: R¹ is F, R² is CH₃, R³ is H, R⁴ is CH₃, R⁵ is CH₃, R⁶ is

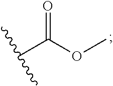 [SMILES: *C(=O)OC]

Number A281: R¹ is F, R² is CH₃, R³ is H, R⁴ is CH₃, R⁵ is CH₃, R⁶ is

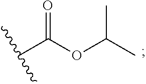 [SMILES: *C(=O)OC(C)C]

Number A282: R¹ is F, R² is CH₃, R³ is H, R⁴ is CH₃, R⁵ is CH₃, R⁶ is

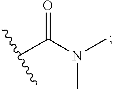 [SMILES: *C(=O)N(C)C]

Number A283: R¹ is F, R² is CH₃, R³ is H, R⁴ is CH₃, R⁵ is CH₃, R⁶ is

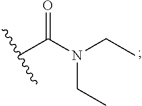 [SMILES: *C(=O)N(CC)CC]

Number A284: R¹ is F, R² is CH₃, R³ is H, R⁴ is CH₃, R⁵ is CH₃, R⁶ is —S(═O)₂CH₃;
Number A285: R¹ is F, R² is CH₃, R³ is H, R⁴ is CH₃, R⁵ is CH₃, R⁶ is —S(═O)₂CH₂CH₃;
Number A286: R¹ is F, R² is CH₃, R³ is H, R⁴ is CH₃, R⁵ is CH₃, R⁶ is —S(═O)₂CH₂CH₂CH₃;
Number A287: R¹ is F, R² is CH₃, R³ is H, R⁴ is CH₃, R⁵ is CH₃, R⁶ is —S(═O)₂CH(CH₃)₂;
Number A288: R¹ is F, R² is CH₃, R³ is H, R⁴ is CH₃, R⁵ is CH₃, R⁶ is —S(═O)₂C₆H₅;
Number A289: R¹ is F, R² is CH₃, R³ is H, R⁴ is CH₃, R⁵ is CH₃, R⁶ is

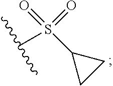 [SMILES: *S(=O)(=O)C1CC1]

Number A290: R¹ is F, R² is CH₃, R³ is H, R⁴ is CH₃, R⁵ is CH₃, R⁶ is

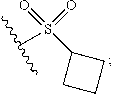 [SMILES: *S(=O)(=O)C1CCC1]

Number A291: R¹ is F, R² is CH₃, R³ is H, R⁴ is CH₃, R⁵ is CH₃, R⁶ is

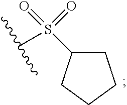 [SMILES: *S(=O)(=O)C1CCCC1]

Number A292: R¹ is F, R² is CH₃, R³ is H, R⁴ is CH₃, R⁵ is CH₃, R⁶ is

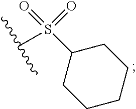 [SMILES: *S(=O)(=O)C1CCCCC1]

Number A293: R¹ is F, R² is CH₃, R³ is CH₃, R⁴ is H, R⁵ is H, R⁶ is

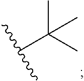 [SMILES: *C(C)(C)C]

Number A294: R¹ is F, R² is CH₃, R³ is OCH₃, R⁴ is H, R⁵ is H, R⁶ is

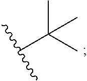 [SMILES: *C(C)(C)C ; ";"]

Number A295: R¹ is F, R² is CH₃, R³ is

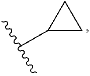 [SMILES: *C1CC1]

R⁴ is H, R⁵ is H, R⁶ is

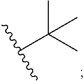 [SMILES: *C(C)(C)C]

Number A296: R¹ is F, R² is CH₃, R³ is COCH₃, R⁴ is H, R⁵ is H, R⁶ is

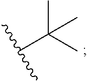 [SMILES: *C(C)(C)C ; ";"]

Number A297: R¹ is F, R² is CH₃, R³ is

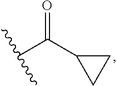 [SMILES: *C(=O)C1CC1]

R⁴ is H, R⁵ is H, R⁶ is

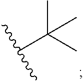 [SMILES: *C(C)(C)C]

Number A298: R¹ is F, R² is CH₃, R³ is

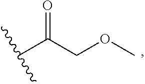 [SMILES: *C(=O)COC]

R⁴ is H, R⁵ is H, R⁶ is

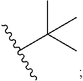 [SMILES: *C(C)(C)C]

Number A299: R¹ is F, R² is CH₃, R³ is

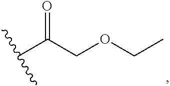 [SMILES: *C(=O)COCC]

R⁴ is H, R⁵ is H, R⁶ is

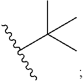 [SMILES: *C(C)(C)C]

Number A300: R¹ is F, R² is CN, R³ is H, R⁴ is H, R⁵ is H, R⁶ is

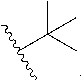 [SMILES: *C(C)(C)C]

The present invention does not require any particular method for preparing the compounds described in the first aspect, and a person skilled in the art can determine a suitable synthetic route to obtain the compounds described in the first aspect of the invention based on the structural formulae provided herein in combination with knowledge known in the art of organic synthesis.

In a second aspect, the present invention provides the use of the compound containing a pyrazole structure according to the first aspect, or an agrochemically acceptable salt, hydrate, and solvate thereof, for at least one of insect killing, mite killing, nematode killing, and piercing-sucking insects killing.

A third aspect of the present invention provides the use of the aforementioned pyrazole structure-containing compound or an agrochemically acceptable salt, hydrate and solvate thereof as an agrochemical insecticide.

A fourth aspect of the present invention provides an insecticide containing a pyrazole structure or an agrochemically acceptable salt, hydrate and solvate thereof as an active ingredient.

Preferably, the content of said active ingredient in the insecticide is 1-99.9% by weight.

more preferably, the content of the active ingredient in the pesticide is 1-99.9 wt %, and may be, for example, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %, 95 wt %, etc.

Particularly preferably, the content of the active ingredient in the insecticide is 5 to 95% by weight.

Preferably, the formulation of the pesticide is at least one selected from the group consisting of emulsifiable concentrate, suspension, wettable powder, dustpowder, granule, aqueous solution, poison bait, mother liquor and mother powder.

Where not specifically recited, all of the following examples are racemates.

The present invention will be described in detail below by way of examples. In the following examples, various raw materials used are commercially available unless otherwise specified.

Unless otherwise specified, the room temperature or the normal temperatures described below means 25±3° C.

The numbering of the compounds provided in the examples section below of the present invention is different from the numbering of the compounds in the preceding, i.e., there are instances where one structural formula has two numbering, which does not affect the clear manner of presentation of the invention. It will be clear that any reference numeral refers to the same structure.

First, the characterization data of some compounds of the present invention are listed in table 1.

TABLE 1

| Compound number | structure | nuclear magnetism |
|---|---|---|
| 1 | 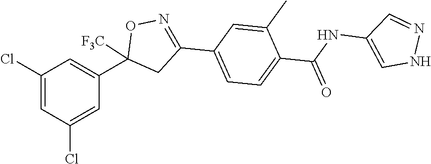 [SMILES: Cc1cc(C2=NOC(c3cc(Cl)cc(Cl)c3)(C(F)(F)F)C2)ccc1C(=O)Nc1cn[nH]c1] | ¹H NMR (600 MHz, DMSO-d₆) δ 12.66 (s, 1H), 10.44 (s, 1H), 7.97 (s, 1H), 7.82 (d, J = 2.3 Hz, 1H), 7.68-7.61 (m, 5H), 7.55 (d, J = 8.0 Hz, 1H), 4.40 (d, J = 18.4 Hz, 1H), 4.32 (d, J = 18.4 Hz, 1H), 2.41 (s, 3H). |
| 2 | 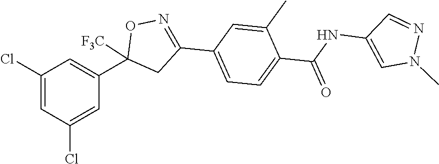 [SMILES: Cc1cc(C2=NOC(c3cc(Cl)cc(Cl)c3)(C(F)(F)F)C2)ccc1C(=O)Nc1cnn(C)c1] | ¹H NMR (600 MHz, DMSO-d₆) δ 10.44 (s, 1H), 8.01 (s, 1H), 7.83 (s, 1H), 7.68-7.61 (m, 4H), 7.55 (d, J = 7.9 Hz, 1H), 7.49 (s, 1H), 4.40 (d, J = 18.4 Hz, 1H), 4.32 (d, J = 18.4 Hz, 1H), 3.82 (s, 3H), 2.41 (s, 3H). |
| 3 | 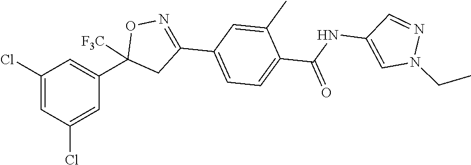 [SMILES: CCn1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.45 (s, 1H), 8.04 (s, 1H), 7.83 (s, 1H), 7.70-7.60 (m, 4H), 7.55 (d, J = 7.9 Hz, 1H), 7.50 (s, 1H), 4.41 (d, J = 18.4 Hz, 1H), 4.33 (d, J = 18.4 Hz, 1H), 4.12 (q, J = 7.3 Hz, 2H), 2.41 (s, 3H), 1.36 (t, J = 7.3 Hz, 3H). |
| 4 | 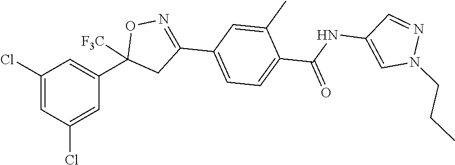 [SMILES: CCCn1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.44 (s, 1H), 8.02 (s, 1H), 7.83 (s, 1H), 7.67-7.62 (m, 4H), 7.55 (d, J = 8.0 Hz, 1H), 7.50 (s, 1H), 4.40 (d, J = 18.4 Hz, 1H), 4.32 (d, J = 18.4 Hz, 1H), 4.04 (t, J = 6.9 Hz, 2H), 2.41 (s, 3H), 1.80 - 1.73 (m, 2H), 0.83 (t, J = 7.4 Hz, 3H). |
| 5 | 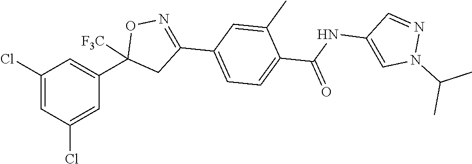 [SMILES: Cc1cc(C2=NOC(c3cc(Cl)cc(Cl)c3)(C(F)(F)F)C2)ccc1C(=O)Nc1cnn(C(C)C)c1] | ¹H NMR (600 MHz, DMSO-d₆) δ 10.43 (s, 1H), 8.03 (s, 1H), 7.83 (s, 1H), 7.68-7.62 (m, 4H), 7.54 (d, J = 7.9 Hz, 1H), 7.50 (s, 1H), 4.53- 4.45 (m, 1H), 4.40 (d, J = 18.4 Hz, 1H), 4.32 (d, J = 18.4 Hz, 1H), 2.41 (s, 3H), 1.41 (d, J = 6.7 Hz, 6H). |
| 6 | 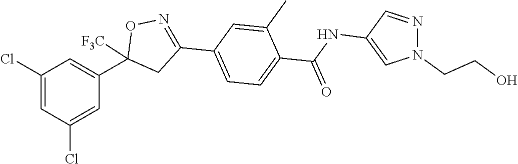 [SMILES: Cc1cc(C2=NOC(c3cc(Cl)cc(Cl)c3)(C(F)(F)F)C2)ccc1C(=O)Nc1cnn(CCO)c1] | ¹H NMR (400 MHz, Chloroform-d) δ 8.14 (s, 1H), 7.77 (s, 1H), 7.56- 7.46 (m, 6H), 7.44 (t, J = 1.9 Hz, 1H), 4.26 (t, J = 4.7 Hz, 2H), 4.10 (d, J = 17.4 Hz, 1H), 4.01 (t, J = 4.7 Hz, 2H), 3.72 (d, J = 17.4 Hz, 1H), 2.49 (s, 3H) , 2.29 (s, 1H). |
| 7 | 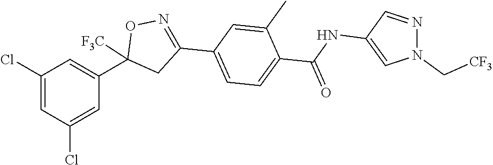 [SMILES: Cc1cc(C2=NOC(c3cc(Cl)cc(Cl)c3)(C(F)(F)F)C2)ccc1C(=O)Nc1cnn(CC(F)(F)F)c1] | ¹H NMR (600 MHz, DMSO-d₆) δ 10.59 (s, 1H), 8.23 (s, 1H), 7.82 (d, J = 2.0 Hz, 1H), 7.69 - 7.61 (m, 5H), 7.59 (d, J = 7.9 Hz, 1H), 5.14 (q, J = 9.1 Hz, 2H), 4.41 (d, J = 18.4 Hz, 1H), 4.33 (d, J = 18.4 Hz, 1H), 2.42 (s, 3H). |
| 8 | 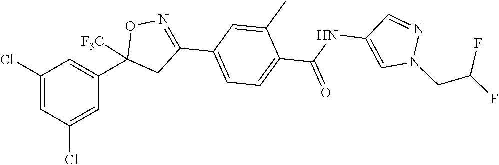 [SMILES: Cc1cc(C2=NOC(c3cc(Cl)cc(Cl)c3)(C(F)(F)F)C2)ccc1C(=O)Nc1cnn(CC(F)F)c1] | ¹H NMR (600 MHz, Chloroform-d) δ 8.16 (s, 1H), 7.62 -7.47 (m, 7H), 7.44 (t, J = 2.0 Hz, 1H), 6.23 - 5.97 (m, 1H), 4.52-4.40 (m, 2H), 4.09 (d, J = 17.4 Hz, 1H), 3.71 (d, J = 17.4 Hz, 1H), 2.52 (s, 3H). |
| 9 | 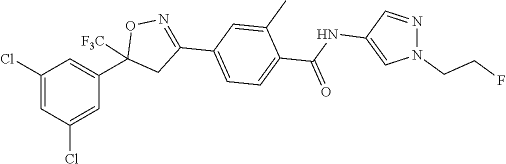 [SMILES: Cc1cc(C2=NOC(c3cc(Cl)cc(Cl)c3)(C(F)(F)F)C2)ccc1C(=O)Nc1cnn(CCF)c1] | ¹H NMR (400 MHz, DMSO-d₆) δ 10.50 (s, 1H), 8.12 (s, 1H), 7.83 (t, J = 1.9 Hz, 1H), 7.70-7.64 (m, 4H), 7.57 (d, J = 7.6 Hz, 2H), 4.83 (t, J = 4.7 Hz, 1H), 4.71 (t, J = 4.7 Hz, 1H), 4.47 (t, J = 4.7 Hz, 1H), 4.45- 4.38 (m, 2H), 4.33 (d, J = 18.4 Hz, 1H), 2.43 (s, 3H). |
| 10 | 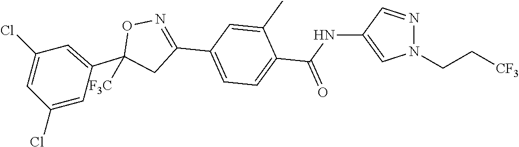 [SMILES: Cc1cc(C2=NOC(c3cc(Cl)cc(Cl)c3)(C(F)(F)F)C2)ccc1C(=O)Nc1cnn(CCC(F)(F)F)c1] | ¹H NMR (400 MHz, Chloroform-d) δ 8.16 (s, 1H), 7.70 (s, 1H), 7.59- 7.50 (m, 6H), 7.47 (t, J = 1.8 Hz, 1H), 4.40 (t, J = 7.2 Hz, 2H), 4.13 (d, J = 17.4 Hz, 1H), 3.75 (d, J = 17.4 Hz, 1H), 2.87-2.71 (m, 2H), 2.53 (s, 3H). |
| 11 | 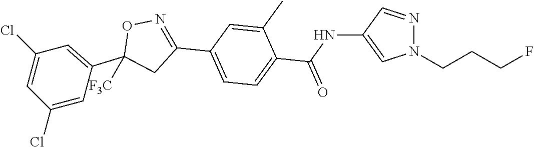 [SMILES: Cc1cc(C2=NOC(c3cc(Cl)cc(Cl)c3)(C(F)(F)F)C2)ccc1C(=O)Nc1cnn(CCCF)c1] | ¹H NMR (400 MHz, Chloroform-d) δ 8.10 (s, 1H), 7.73 (s, 1H), 7.56- 7.47 (m, 6H), 7.44 (t, J = 1.9 Hz, 1H), 4.52 (t, J = 5.6 Hz, 1H), 4.40 (t, J = 5.6 Hz, 1H), 4.27 (t, J = 6.8 Hz, 2H), 4.10 (d, J = 17.4 Hz, 1H), 3.72 (d, J = 17.4 Hz, 1H), 2.50 (s, 3H), 2.36-2.18 (m, 2H). |
| 12 | 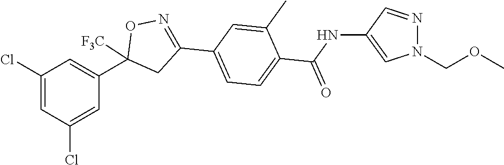 [SMILES: COCn1cc(NC(=O)c2ccc(C3=NOC(c4cc(Cl)cc(Cl)c4)(C(F)(F)F)C3)cc2C)cn1] | ¹H NMR (600 MHZ, Chloroform-d) δ 8.24 (s, 1H), 7.60-7.50 (m, 7H), 7.44 (s, 1H), 5.38 (s, 2H), 4.10 (d, J = 17.4 Hz, 1H), 3.71 (d, J = 17.4 Hz, 1H), 3.35 (s, 3H), 2.52 (s, 3H). |
| 13 | 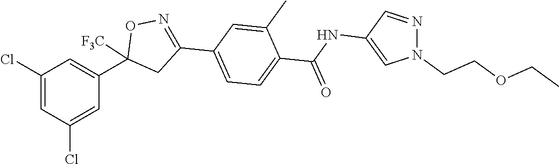 [SMILES: CCOCCn1cc(NC(=O)c2ccc(C3=NOC(c4cc(Cl)cc(Cl)c4)(C(F)(F)F)C3)cc2C)cn1] | ¹H NMR (400 MHz, DMSO-d₆) δ 10.46 (s, 1H), 8.05 (s, 1H), 7.84 (q, J = 2.1 Hz, 1H), 7.70-7.64 (m, 4H), 7.60-7.53 (m, 2H), 4.43 (d, J = 18.4 Hz, 1H), 4.34 (d, J = 18.4 Hz, 1H), 4.25 (t, J = 5.4 Hz, 2H), 3.73 (t, J = 5.4 Hz, 2H), 3.45 (q, J = 7.0 Hz, 2H), 2.43 (s, 3H), 1.10 (t, J = 7.0 Hz, 3H). |
| 14 | 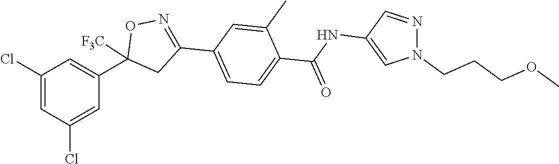 [SMILES: COCCCn1cc(NC(=O)c2ccc(C3=NOC(c4cc(Cl)cc(Cl)c4)(C(F)(F)F)C3)cc2C)cn1] | ¹H NMR (400 MHz, DMSO-d₆) δ 10.46 (s, 1H), 8.02 (d, J = 0.7 Hz, 1H), 7.83 (t, J = 1.9 Hz, 1H), 7.69-7.62 (m, 4H), 7.56 (d, J = 7.9 Hz, 1H), 7.53 (d, J = 0.8 Hz, 1H), 4.41 (d, J = 18.4 Hz, 1H), 4.32 (d, J = 18.4 Hz, 1H), 4.13 (t, J = 7.0 Hz, 2H), 3.29 (t, J = 6.2 Hz, 2H), 3.24 (s, 3H), 2.41 (s, 3H), 2.02-1.94 (m, 2H). |
| 15 | 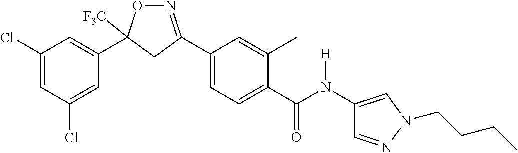 [SMILES: CCCCn1cc(NC(=O)c2ccc(C3=NOC(c4cc(Cl)cc(Cl)c4)(C(F)(F)F)C3)cc2C)cn1] | ¹H NMR (400 MHz, Chloroform-d) δ 8.08 (s, 1H), 8.05 (s, 1H), 7.51 (d, J = 1.9 Hz, 2H), 7.48 (d, J = 3.4 Hz, 1H), 7.46-7.41 (m, 4H), 4.13- 4.04 (m, 3H), 3.72 (d, J = 17.4 Hz, 1H), 2.46 (s, 3H), 1.88-1.79 (m, 2H), 1.40-1.28 (m, 2H), 0.93 (t, J = 7.4 Hz, 3H). |
| 16 | 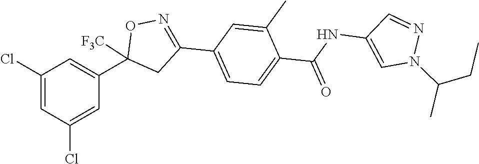 [SMILES: CCC(C)n1cc(NC(=O)c2ccc(C3=NOC(c4cc(Cl)cc(Cl)c4)(C(F)(F)F)C3)cc2C)cn1] | ¹H NMR (400 MHz, Chloroform-d) δ 8.12 (s, 1H), 7.76 (s, 1H), 7.53 (d, J = 9.7 Hz, 6H), 7.44 (d, J = 1.9 Hz, 1H), 4.28-4.19 (m, 1H), 4.10 (d, J = 17.4 Hz, 1H), 3.72 (d, J = 17.4 Hz, 1H), 2.52 (s, 3H), 1.99- 1.74 (m, 2H), 1.51 (d, J = 6.8 Hz, 3H), 0.84 (t, J = 7.4 Hz, 3H). |
| 17 | 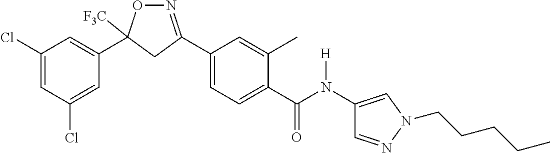 [SMILES: CCCCCn1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.42 (s, 1H), 8.00 (d, J = 2.8 Hz, 1H), 7.80 (s, 1H), 7.63 (d, J = 13.1 Hz, 4H), 7.55-7.51 (m, 1H), 7.48 (d, J = 3.0 Hz, 1H), 4.38 (d, J = 18.4 Hz, 1H), 4.30 (d, J = 18.4 Hz, 1H), 4.04 (t, J = 7.0 Hz, 2H), 2.39 (s, 3H), 1.76 - 1.69 (m, 2H), 1.32- 1.21 (m, 2H), 1.22-1.16 (m, 2H), 0.83 (t, J = 7.5 Hz, 3H). |
| 18 | 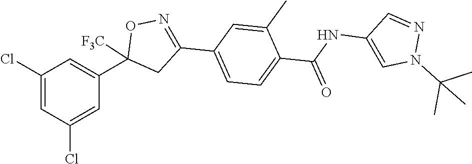 [SMILES: Cc1cc(C2=NOC(c3cc(Cl)cc(Cl)c3)(C(F)(F)F)C2)ccc1C(=O)Nc1cnn(C(C)(C)C)c1] | ¹H NMR (400 MHz, DMSO-d₆) δ 10.43 (s, 1H), 8.06 (s, 1H), 7.83 (t, J = 1.9 Hz, 1H), 7.68-7.62 (m, 4H), 7.54 (d, J = 6.9 Hz, 2H), 4.41 (d, J = 18.4 Hz, 1H), 4.32 (d, J = 18.4 Hz, 1H), 2.41 (s, 3H), 1.52 (s, 9H). |
| 19 | 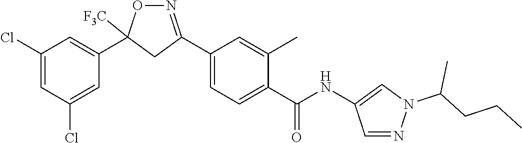 [SMILES: CCCC(C)n1cc(NC(=O)c2ccc(C3=NOC(c4cc(Cl)cc(Cl)c4)(C(F)(F)F)C3)cc2C)cn1] | ¹H NMR (400 MHz, Chloroform-d) δ 8.13 (s, 1H), 7.73-7.65 (m, 1H), 7.58-7.48 (m, 6H), 7.44 (t, J = 1.9 Hz, 1H), 4.40-4.26 (m, 1H), 4.10 (d, J = 17.4 Hz, 1H), 3.71 (d, J = 17.4 Hz, 1H), 2.53 (s, 3H), 1.94- 1.86 (m, 1H), 1.81-1.64 (m, 1H), 1.51 (d, J = 6.7 Hz, 3H), 1.37- 1.13 (m, 2H), 0.91 (t, J = 7.3 Hz, 3H). |
| 20 | 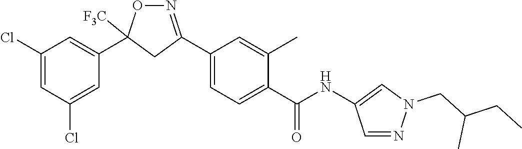 [SMILES: CCC(C)Cn1cc(NC(=O)c2ccc(C3=NOC(c4cc(Cl)cc(Cl)c4)(C(F)(F)F)C3)cc2C)cn1] | ¹H NMR (400 MHz, DMSO-d₆) δ 10.47 (s, 1H), 8.03 (s, 1H), 7.87- 7.82 (m, 1H), 7.71-7.64 (m, 4H), 7.58 (d, J = 7.9 Hz, 1H), 7.53 (d, J = 1.1 Hz, 1H), 4.43 (d, J = 18.4 Hz, 1H), 4.34 (d, J = 18.4 Hz, 1H), 4.06-3.97 (m, 1H), 3.95 - 3.85 (m, 1H), 2.44 (s, 3H), 1.96-1.86 (m, 1H), 1.38-1.24 (m, 1H), 1.20-1.06 (m, 1H), 0.89 (t, J = 7.4 Hz, 3H), 0.82 (d, J = 6.7 Hz, 3H). |
| 21 | 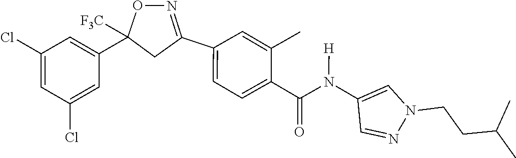 [SMILES: Cc1cc(C2=NOC(c3cc(Cl)cc(Cl)c3)(C(F)(F)F)C2)ccc1C(=O)Nc1cnn(CCC(C)C)c1] | ¹H NMR (400 MHz, Chloroform-d) δ 8.11 (s, 1H), 7.71 (s, 1H), 7.58- 7.47 (m, 6H), 7.46-7.41 (m, 1H), 4.18 - 4.06 (m, 3H), 3.71 (d, J = 17.4 Hz, 1H), 2.51 (s, 3H), 1.83-1.74 (m, 2H), 1.68-1.53 (m, 1H), 0.96 (d, J = 6.6 Hz, 6H). |
| 22 | 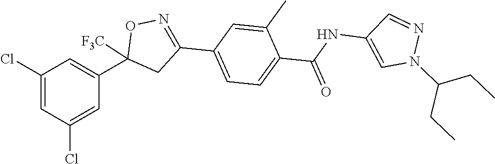 [SMILES: CCC(CC)n1cc(NC(=O)c2ccc(C3=NOC(c4cc(Cl)cc(Cl)c4)(C(F)(F)F)C3)cc2C)cn1] | ¹H NMR (600 MHz, Chloroform-d) δ 8.09 (s, 1H), 7.59-7.46 (m, 7H), 7.43 (t, J = 1.9 Hz, 1H), 4.09 (d, J = 17.4 Hz, 1H), 3.96-3.83 (m, 1H), 3.71 (d, J = 17.4 Hz, 1H), 2.54 (s, 3H), 1.95-1.77 (m, 4H), 0.81 (t, J = 7.3 Hz, 6H). |
| 23 | 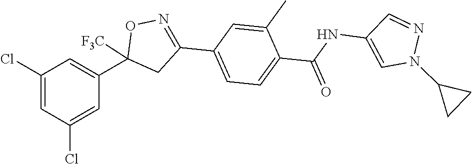 [SMILES: Cc1cc(C2=NOC(c3cc(Cl)cc(Cl)c3)(C(F)(F)F)C2)ccc1C(=O)Nc1cnn(C2CC2)c1] | ¹H NMR (600 MHz, DMSO-d₆) δ 10.41 (s, 1H), 8.01 (s, 1H), 7.80 (d, J = 1.7 Hz, 1H), 7.65-7.60 (m, 4H), 7.51 (d, J = 7.9 Hz, 1H), 7.46 (s, 1H), 4.37 (d, J = 18.4 Hz, 1H), 4.29 (d, J = 18.4 Hz, 1H), 3.72-3.66 (m, 1H), 2.38 (s, 3H), 1.02-0.98 (m, 2H), 0.94-0.89 (m, 2H). |
| 24 | 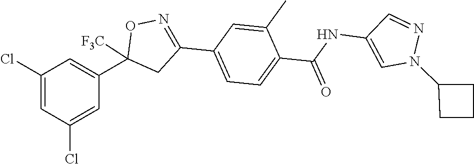 [SMILES: Cc1cc(C2=NOC(c3cc(Cl)cc(Cl)c3)(C(F)(F)F)C2)ccc1C(=O)Nc1cnn(C2CCC2)c1] | ¹H NMR (600 MHz, DMSO-d₆) δ 10.45 (s, 1H), 8.07 (s, 1H), 7.83 (s, 1H), 7.69-7.60 (m, 4H), 7.54 (d, J = 7.8 Hz, 2H), 4.86-4.78 (m, 1H), 4.40 (d, J = 18.4 Hz, 1H), 4.32 (d, J = 18.4 Hz, 1H), 2.47-2.42 (m, 2H), 2.40 (s, 3H), 2.37-2.33 (m, 2H), 1.82-1.70 (m, 2H). |
| 25 | 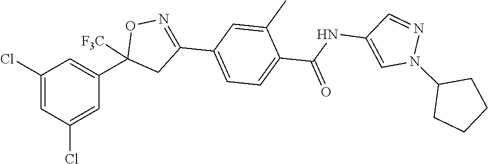 [SMILES: Cc1cc(C2=NOC(c3cc(Cl)cc(Cl)c3)(C(F)(F)F)C2)ccc1C(=O)Nc1cnn(C2CCCC2)c1] | ¹H NMR (600 MHz, DMSO-d₆) δ 10.42 (s, 1H), 8.00 (s, 1H), 7.81 (s, 1H), 7.66-7.57 (m, 4H), 7.52 (d, J = 7.9 Hz, 1H), 7.48 (s, 1H), 4.69- 4.63 (m, 1H), 4.38 (d, J = 18.4 Hz, 1H), 4.30 (d, J = 18.4 Hz, 1H), 2.38 (s, 3H), 2.08-2.00 (m, 2H), 1.92-1.84 (m, 2H), 1.80-1.71 (m, 2H), 1.67-1.57 (m, 2H). |
| 26 | 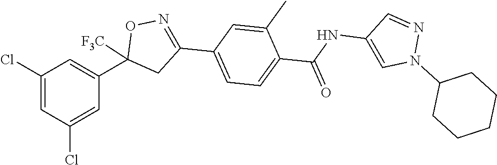 [SMILES: Cc1cc(C2=NOC(c3cc(Cl)cc(Cl)c3)(C(F)(F)F)C2)ccc1C(=O)Nc1cnn(C2CCCCC2)c1] | ¹H NMR (600 MHz, DMSO-d₆) δ 10.44 (s, 1H), 8.01 (s, 1H), 7.83 (s, 1H), 7.68-7.62 (m, 4H), 7.54 (d, J = 7.9 Hz, 1H), 7.51 (s, 1H), 4.40 (d, J = 18.4 Hz, 1H), 4.32 (d, J = 18.4 Hz, 1H), 4.15-4.08 (m, 1H), 2.41 (s, 3H), 2.03-1.95 (m, 2H), 1.84-1.77 (m, 2H), 1.74-1.67 (m, 2H), 1.43-1.34 (m, 2H), 1.26-1.19 (m, 2H). |
| 27 | 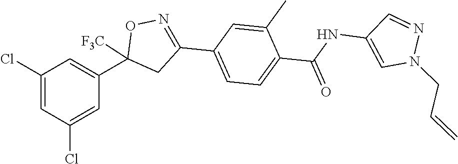 [SMILES: C=CCn1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.47 (s, 1H), 8.03 (s, 1H), 7.83 (s, 1H), 7.68-7.61 (m, 4H), 7.58-7.51 (m, 2H), 6.05-5.96 (m, 1H), 5.23-5.12 (m, 2H), 4.74 (d, J = 4.2 Hz, 2H), 4.40 (d, J = 18.4 Hz, 1H), 4.32 (d, J = 18.4 Hz, 1H), 2.41 (s, 3H). |
| 28 | 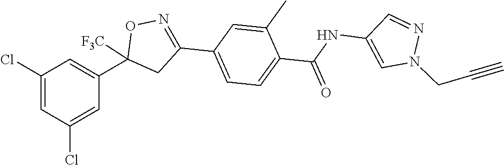 [SMILES: C#CCn1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.59 (s, 1H), 8.23 (s, 1H), 7.82 (s, 1H), 7.67 (s, 2H), 7.65 (s, 1H), 7.64 (s, 2H), 7.57 (d, J = 7.9 Hz, 1H), 5.48 (s, 2H), 4.41 (d, J = 18.4 Hz, 1H), 4.32 (d, J = 18.4 Hz, 1H), 2.61 (s, 1H), 2.42 (s, 3H). |
| 29 | 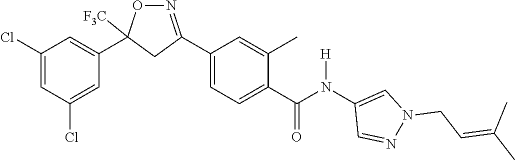 [SMILES: CC(C)=CCn1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.46 (s, 1H), 8.00 (s, 1H), 7.82 (s, 1H), 7.66 (d, J = 12.0 Hz, 4H), 7.56 (d, J = 7.8 Hz, 1H), 7.50 (s, 1H), 5.39 (t, J = 7.3 Hz, 1H), 4.70 (d, J = 7.1 Hz, 2H), 4.41 (d, J = 18.4 Hz, 1H), 4.33 (d, J = 18.4 Hz, 1H), 2.42 (s, 3H), 1.75 (d, J = 10.7 Hz, 6H). |
| 30 | 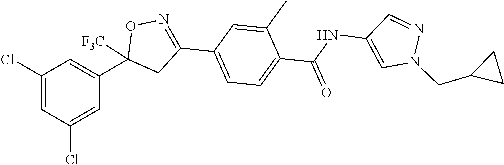 [SMILES: Cc1cc(C2=NOC(c3cc(Cl)cc(Cl)c3)(C(F)(F)F)C2)ccc1C(=O)Nc1cnn(CC2CC2)c1] | ¹H NMR (600 MHz, DMSO-d₆) δ 10.46 (s, 1H), 8.08 (s, 1H), 7.83 (d, J = 2.1 Hz, 1H), 7.68-7.62 (m, 4H), 7.55 (d, J = 7.8 Hz, 2H), 4.86- 4.79 (m, 1H), 4.41 (d, J = 18.4 Hz, 1H), 4.33 (d, J = 18.4 Hz, 1H), 2.45 (d, J = 7.3 Hz, 2H), 2.41 (s, 3H), 2.39-2.32 (m, 2H), 1.82-1.72 (m, 2H). |
| 31 | 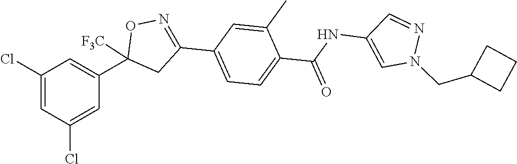 [SMILES: Cc1cc(C2=NOC(c3cc(Cl)cc(Cl)c3)(C(F)(F)F)C2)ccc1C(=O)Nc1cnn(CC2CCC2)c1] | ¹H NMR (600 MHz, DMSO-d₆) δ 10.44 (s, 1H), 8.00 (s, 1H), 7.83 (s, 1H), 7.67-7.61 (m, 4H), 7.55 (d, J = 7.8 Hz, 1H), 7.50 (s, 1H), 4.40 (d, J = 18.4 Hz, 1H), 4.32 (d, J = 18.4 Hz, 1H), 4.10 (d, J = 7.3 Hz, 2H), 2.77-2.66 (m, 1H), 2.41 (s, 3H), 2.03-1.92 (m, 2H), 1.88- 1.80 (m, 2H), 1.80-1.69 (m, 2H). |
| 32 | 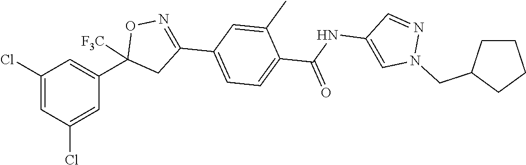 [SMILES: Cc1cc(C2=NOC(c3cc(Cl)cc(Cl)c3)(C(F)(F)F)C2)ccc1C(=O)Nc1cnn(CC2CCCC2)c1] | ¹H NMR (600 MHz, DMSO-d₆) δ 10.40 (s, 1H), 8.00 (s, 1H), 7.80 (s, 1H), 7.63 (s, 1H), 7.61 (s, 3H), 7.53 (d, J = 7.7 Hz, 1H), 7.47 (s, 1H), 4.38 (d, J = 18.4 Hz, 1H), 4.29 (d, J = 18.4 Hz, 1H), 3.97 (d, J = 7.2 Hz, 2H), 2.39 (s, 3H), 1.99-1.94 (m, 1H), 1.61-1.55 (m, 4H), 1.51-1.45 (m, 2H), 1.25-1.20 (m, 2H). |
| 33 | 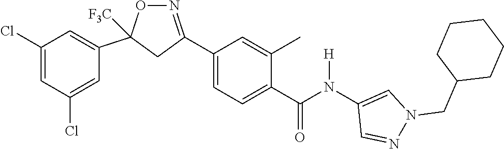 [SMILES: Cc1cc(C2=NOC(c3cc(Cl)cc(Cl)c3)(C(F)(F)F)C2)ccc1C(=O)Nc1cnn(CC2CCCCC2)c1] | ¹H NMR (400 MHz, Chloroform-d) δ 8.10 (s, 1H), 7.74 (s, 1H), 7.55 (d, J = 4.5 Hz, 4H), 7.53-7.50 (m, 2H), 7.44 (q, J = 1.6 Hz, 1H), 4.10 (d, J = 17.4 Hz, 1H), 3.96 (d, J = 7.2 Hz, 2H), 3.71 (d, J = 17.4 Hz, 1H), 2.53 (s, 3H), 1.95-1.85 (m, 1H), 1.78-1.59 (m, 4H), 1.34- 1.10 (m, 4H), 1.06-0.91 (m, 2H). |
| 34 | 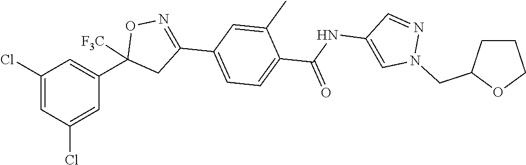 [SMILES: Cc1cc(C2=NOC(c3cc(Cl)cc(Cl)c3)(C(F)(F)F)C2)ccc1C(=O)Nc1cnn(CC2CCCO2)c1] | ¹H NMR (600 MHz, DMSO-d₆) δ 10.44 (s, 1H), 8.03 (s, 1H), 7.83 (d, J = 2.4 Hz, 1H), 7.66 (d, J = 4.7 Hz, 1H), 7.64 (s, 3H), 7.56 (d, J = 7.9 Hz, 1H), 7.52 (s, 1H), 4.41 (d, J = 18.4 Hz, 1H), 4.32 (d, J = 18.4 Hz, 1H), 4.17-4.08 (m, 3H), 3.76 (q, J = 7.2 Hz, 1H), 3.64 (q, J = 7.2 Hz, 1H), 2.42 (s, 3H), 1.95-1.87 (m, 1H), 1.82-1.75 (m, 2H), 1.61- 1.54 (m, 1H). |
| 35 | 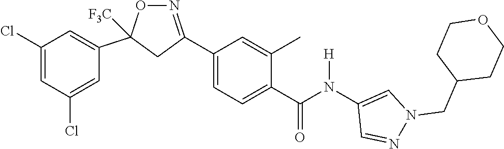 [SMILES: Cc1cc(C2=NOC(c3cc(Cl)cc(Cl)c3)(C(F)(F)F)C2)ccc1C(=O)Nc1cnn(CC2CCOCC2)c1] | ¹H NMR (400 MHz, DMSO-d₆) δ 10.45 (s, 1H), 8.03 (s, 1H), 7.82 (s, 1H), 7.69-7.60 (m, 4H), 7.59-7.48 (m, 2H), 4.40 (d, J = 18.4 Hz, 1H), 4.32 (d, J = 18.4 Hz, 1H), 4.05-3.94 (m, 2H), 3.88-3.76 (m, 2H), 3.30-3.18 (m, 2H), 2.41 (s, 3H), 2.01-1.96 (m, 1H), 1.46- 1.32 (m, 2H), 1.30-1.18 (m, 2H). |
| 36 | 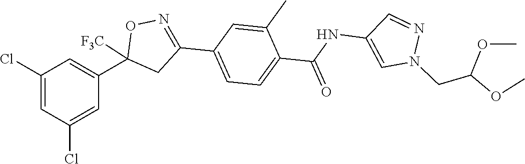 [SMILES: COC(Cn1cc(NC(=O)c2ccc(C3=NOC(c4cc(Cl)cc(Cl)c4)(C(F)(F)F)C3)cc2C)cn1)OC] | ¹H NMR (600 MHz, DMSO-d₆) δ 10.45 (s, 1H), 8.03 (s, 1H), 7.83 (s, 1H), 7.65 (d, J = 13.0 Hz, 4H), 7.56 (d, J = 8.2 Hz, 1H), 7.54 (s, 1H), 4.69 (t, J = 5.4 Hz ,1H), 4.40 (d, J = 18.4 Hz, 1H), 4.32 (d, J = 18.4 Hz, 1H), 4.20 (d, J = 5.4 Hz, 2H), 3.28 (s, 6H), 2.41 (s, 3H). |
| 37 | 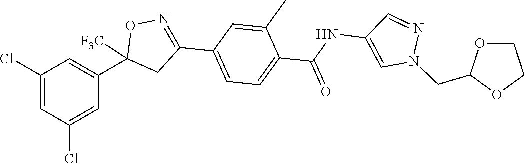 [SMILES: Cc1cc(C2=NOC(c3cc(Cl)cc(Cl)c3)(C(F)(F)F)C2)ccc1C(=O)Nc1cnn(CC2OCCO2)c1] | ¹H NMR (600 MHz, DMSO-d₆) δ 10.45 (s, 1H), 8.03 (s, 1H), 7.81 (s, 1H), 7.65 (d, J = 4.4 Hz, 2H), 7.63 (s, 2H), 7.54 (d, J = 7.9 Hz, 1H), 7.52 (s, 1H), 5.13 (t, J = 4.4 Hz, 1H), 4.39 (d, J = 18.4 Hz, 1H), 4.31 (d, J = 18.4 Hz, 1H), 4.21 (d, J = 4.4 Hz, 2H), 3.87 (d, J = 4.7 Hz, 2H), 3.83-3.79 (m, 2H), 2.40 (s, 3H). |
| 38 | 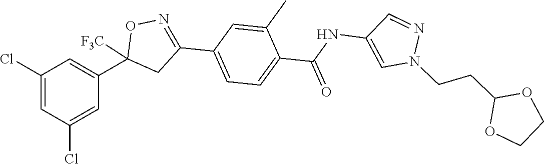 [SMILES: Cc1cc(C2=NOC(c3cc(Cl)cc(Cl)c3)(C(F)(F)F)C2)ccc1C(=O)Nc1cnn(CCC2OCCO2)c1] | ¹H NMR (400 MHz, DMSO-d₆) δ 10.47 (s, 1H), 8.05 (s, 1H), 7.84 (t, J = 1.9 Hz, 1H), 7.70-7.63 (m, 4H), 7.56 (d, J = 13.1 Hz, 2H), 4.83 (t, J = 4.7 Hz, 1H), 4.43 (d, J = 18.4 Hz, 1H), 4.34 (d, J = 18.4 Hz, 1H), 4.19 (t, J = 4.7 Hz, 2H), 3.96-3.90 (m, 2H), 3.83-3.76 (m, 2H), 2.43 (s, 3H), 2.15-2.06 (m, 2H). |
| 39 | 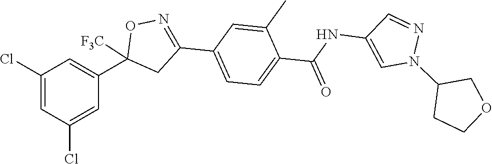 [SMILES: Cc1cc(C2=NOC(c3cc(Cl)cc(Cl)c3)(C(F)(F)F)C2)ccc1C(=O)Nc1cnn(C2CCOC2)c1] | ¹H NMR (400 MHz, DMSO-d₆) δ 10.48 (s, 1H), 8.08 (s, 1H), 7.83 (t, J = 1.9 Hz, 1H), 7.68-7.62 (m, 4H), 7.55 (d, J = 6.1 Hz, 2H), 5.07- 4.98 (m, 1H), 4.41 (d, J = 18.4 Hz, 1H), 4.32 (d, J = 18.4 Hz, 1H), 4.01- 3.92 (m, 2H), 3.90-3.78 (m, 2H), 2.41 (s, 3H), 2.40-2.32 (m, 1H), 2.28-2.18 (m, 1H). |
| 40 | 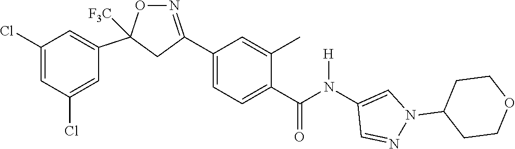 [SMILES: Cc1cc(C2=NOC(c3cc(Cl)cc(Cl)c3)(C(F)(F)F)C2)ccc1C(=O)Nc1cnn(C2CCOCC2)c1] | ¹H NMR (400 MHz, Chloroform-d) δ 8.21 (s, 1H), 7.84 (s, 1H), 7.60 (s, 1H), 7.58-7.52 (m, 3H), 7.51 (d, J = 1.9 Hz, 2H), 7.44 (t, J = 1.9 Hz, 1H), 4.48-4.34 (m, 1H), 4.17-4.04 (m, 3H), 3.72 (d, J = 17.4 Hz, 1H), 3.61-3.48 (m, 2H), 2.52 (s, 3H), 2.17-2.07 (m, 4H). |
| 41 | 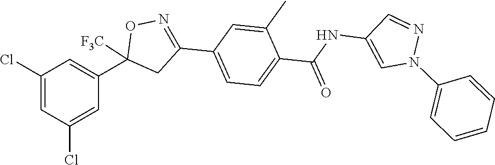 [SMILES: Cc1cc(C2=NOC(c3cc(Cl)cc(Cl)c3)(C(F)(F)F)C2)ccc1C(=O)Nc1cnn(-c2ccccc2)c1] | ¹H NMR (600 MHz, DMSO-d₆) δ 10.68 (s, 1H), 8.67 (s, 1H), 7.88- 7.79 (m, 4H), 7.74-7.59 (m, 5H), 7.50 (t, J = 7.8 Hz, 2H), 7.31 (t, J = 7.4 Hz, 1H), 4.42 (d, J = 18.4 Hz, 1H), 4.33 (d, J = 18.4 Hz, 1H), 2.45 (s, 3H). |
| 42 | 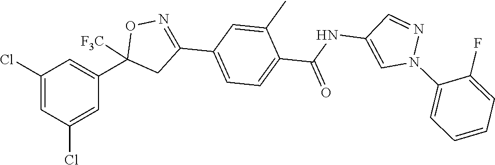 [SMILES: Cc1cc(C2=NOC(c3cc(Cl)cc(Cl)c3)(C(F)(F)F)C2)ccc1C(=O)Nc1cnn(-c2ccccc2F)c1] | ¹H NMR (400 MHz, DMSO-d₆) δ 10.71 (s, 1H), 8.51 (d, J = 2.9 Hz, 1H), 7.91 (s, 1H), 7.86-7.82 (m, 2H), 7.69 (s, 1H), 7.68-7.60 (m, 4H), 7.52-7.34 (m, 3H), 4.41 (d, J = 18.4 Hz, 1H), 4.33 (d, J = 18.4 Hz, 1H), 2.45 (s, 3H). |
| 43 | 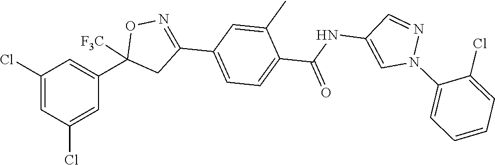 [SMILES: Cc1cc(C2=NOC(c3cc(Cl)cc(Cl)c3)(C(F)(F)F)C2)ccc1C(=O)Nc1cnn(-c2ccccc2Cl)c1] | ¹H NMR (400 MHz, DMSO-d₆) δ 10.52 (s, 1H), 8.14 (s, 1H), 7.83 (t, J = 1.9 Hz, 1H), 7.68-7.61 (m, 4H), 7.61-7.54 (m, 2H), 7.53-7.48 (m, 1H), 7.39-7.31 (m, 2H), 7.06-7.02 (m, 1H), 4.41 (d, J = 18.4 Hz, 1H), 4.32 (d, J = 18.4 Hz, 1H), 2.41 (s, 3H). |
| 44 | 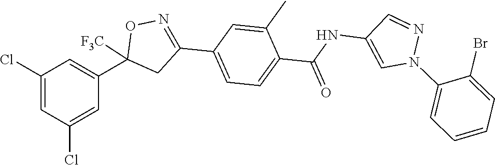 [SMILES: Cc1cc(C2=NOC(c3cc(Cl)cc(Cl)c3)(C(F)(F)F)C2)ccc1C(=O)Nc1cnn(-c2ccccc2Br)c1] | ¹H NMR (600 MHz, DMSO-d₆) δ 10.49 (s, 1H), 8.13 (s, 1H), 7.81 (s, 1H), 7.69-7.59 (m, 6H), 7.57 (d, J = 7.9 Hz, 1H), 7.38 (t, J = 7.5 Hz, 1H), 7.28 (t, J = 7.7 Hz, 1H), 6.99 (d, J = 7.7 Hz, 1H), 4.40 (d, J = 18.4 Hz, 1H), 4.31 (d, J = 18.4 Hz, 1H), 2.42 (s, 3H). |
| 45 | 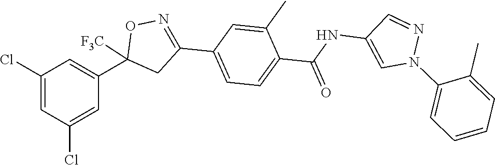 [SMILES: Cc1cc(C2=NOC(c3cc(Cl)cc(Cl)c3)(C(F)(F)F)C2)ccc1C(=O)Nc1cnn(-c2ccccc2C)c1] | ¹H NMR (600 MHz, DMSO-d₆) δ 10.45 (s, 1H), 8.00 (s, 1H), 7.84 7.79 (m, 1H), 7.69-7.59 (m, 4H), 7.55 (d, J = 8.1 Hz, 2H), 7.25- 7.14 (m, 3H), 7.01 (d, J = 7.5 Hz, 1H), 4.39 (d, J = 18.4 Hz, 1H), 4.31 (d, J = 18.4 Hz, 1H), 2.41 (s, 3H), 2.31 (s, 3H). |
| 46 | 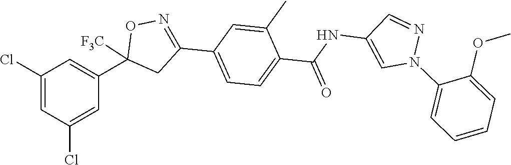 [SMILES: COc1ccccc1-n1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.54 (s, 1H), 8.14 (s, 1H), 7.93- 7.90 (m, 1H), 7.81 (d, J = 3.8 Hz, 1H), 7.65 (t, J = 12.7 Hz, 5H), 7.59 (d, J = 7.3 Hz, 1H), 7.41-7.36 (m, 1H), 7.11-7.06 (m, 1H), 6.92 (d, J = 7.5 Hz, 1H), 4.42 (d, J = 18.3 Hz, 1H), 4.33 (d, J = 18.3 Hz, 1H), 3.69 (s, 3H), 2.43 (s, 3H). |
| 47 | 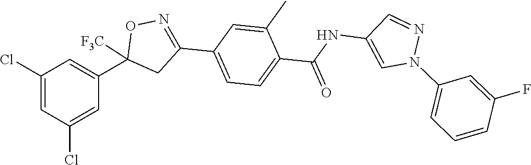 [SMILES: Cc1cc(C2=NOC(c3cc(Cl)cc(Cl)c3)(C(F)(F)F)C2)ccc1C(=O)Nc1cnn(-c2cccc(F)c2)c1] | ¹H NMR (600 MHz, DMSO-d₆) δ 10.73 (s, 1H), 8.76 (s, 1H), 7.88 (s, 1H), 7.82 (t, J = 1.9 Hz, 1H), 7.76-7.67 (m, 4H), 7.65 (d, J = 2.0 Hz, 2H), 7.63 (d, J = 8.0 Hz, 1H), 7.56-7.50 (m, 1H), 7.17-7.11 (m, 1H), 4.43 (d, J = 18.4 Hz, 1H), 4.34 (d, J = 18.4 Hz, 1H), 2.46 (s, 3H). |
| 48 | 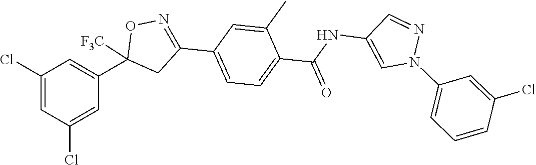 [SMILES: Cc1cc(C2=NOC(c3cc(Cl)cc(Cl)c3)(C(F)(F)F)C2)ccc1C(=O)Nc1cnn(-c2cccc(Cl)c2)c1] | ¹H NMR (400 MHz, DMSO-d₆) δ 10.50 (s, 1H), 8.19 (s, 1H), 7.83 (t, J = 1.9 Hz, 1H), 7.70-7.60 (m, 4H), 7.60-7.53 (m, 2H), 7.43-7.34 (m, 2H), 7.32 (d, J = 2.3 Hz, 1H), 7.28-7.19 (m, 1H), 4.41 (d, J = 18.4 Hz, 1H), 4.32 (d, J = 18.4 Hz, 1H), 2.41 (s, 3H). |
| 49 | 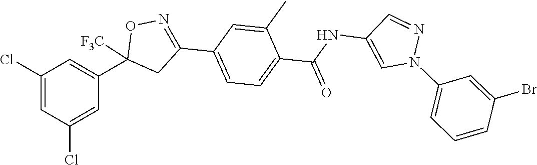 [SMILES: Cc1cc(C2=NOC(c3cc(Cl)cc(Cl)c3)(C(F)(F)F)C2)ccc1C(=O)Nc1cnn(-c2cccc(Br)c2)c1] | ¹H NMR (600 MHz, DMSO-d₆) δ 10.52 (d, J = 6.0 Hz, 1H), 8.24- 8.18 (m, 1H), 7.81 (d, J = 9.9 Hz, 1H), 7.67 (s, 1H), 7.65 (s, 3H), 7.61- 7.56 (m, 2H), 7.50 (d, J = 7.9 Hz, 1H), 7.48 (s, 1H), 7.33 (t, J = 7.6 Hz, 1H), 7.27 (d, J = 7.7 Hz, 1H), 4.41 (d, J = 18.4 Hz, 1H), 4.33 (d, J = 18.4 Hz, 1H), 2.42 (s, 3H). |
| 50 | 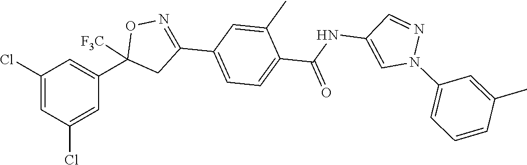 [SMILES: Cc1cccc(-n2cc(NC(=O)c3ccc(C4=NOC(c5cc(Cl)cc(Cl)c5)(C(F)(F)F)C4)cc3C)cn2)c1] | ¹H NMR (600 MHz, DMSO-d₆) δ 10.52 (s, 1H), 8.16 (s, 1H), 7.79 (s, 1H), 7.69 (s, 1H), 7.67 (s, 3H), 7.60 (d, J = 4.3 Hz, 1H), 7.58 (s, 1H), 7.23 (d, J = 7.7 Hz, 1H), 7.11 (d, J = 8.1 Hz, 2H), 7.08 (d, J = 8.0 Hz, 1H), 4.43 (d, J = 18.3 Hz, 1H), 4.34 (d, J = 18.3 Hz, 1H), 2.44 (s, 3H), 2.29 (s, 3H). |
| 51 | 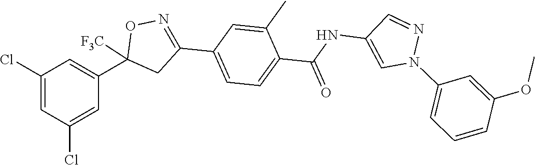 [SMILES: COc1cccc(-n2cc(NC(=O)c3ccc(C4=NOC(c5cc(Cl)cc(Cl)c5)(C(F)(F)F)C4)cc3C)cn2)c1] | ¹H NMR (600 MHz, DMSO-d₆) δ 10.47 (s, 1H), 8.12 (d, J = 2.6 Hz, 1H), 7.79-7.74 (m, 1H), 7.63 (d, J = 13.1 Hz, 4H), 7.57-7.52 (m, 2H), 7.24 (t, J = 7.9 Hz, 1H), 6.83 (s, 2H), 6.80 (d, J = 7.4 Hz, 1H), 4.38 (d, J = 18.4 Hz, 1H), 4.30 (d, J = 18.4 Hz, 1H), 3.71 (s, 3H), 2.40 (s, 3H). |
| 52 | 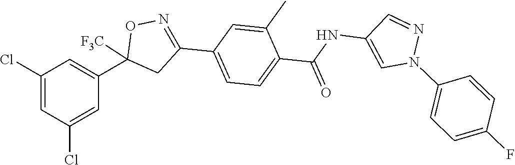 [SMILES: Cc1cc(C2=NOC(c3cc(Cl)cc(Cl)c3)(C(F)(F)F)C2)ccc1C(=O)Nc1cnn(-c2ccc(F)cc2)c1] | ¹H NMR (600 MHz, DMSO-d₆) δ 10.70 (s, 1H), 8.67 (s, 1H), 7.90- 7.85 (m, 2H), 7.85 (s, 1H), 7.83 (s, 1H), 7.69 (d, J = 10.1 Hz, 2H), 7.65 (d, J = 1.9 Hz, 2H), 7.62 (d, J = 7.8 Hz, 1H), 7.34 (t, J = 8.6 Hz, 2H), 4.42 (d, J = 18.4 Hz, 1H), 4.34 (d, J = 18.4 Hz, 1H), 2.46 (s, 3H). |
| 53 | 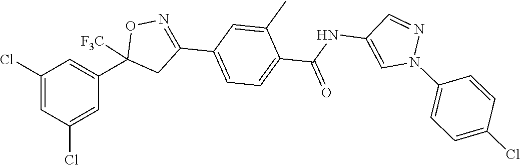 [SMILES: Cc1cc(C2=NOC(c3cc(Cl)cc(Cl)c3)(C(F)(F)F)C2)ccc1C(=O)Nc1cnn(-c2ccc(Cl)cc2)c1] | ¹H NMR (600 MHz, DMSO-d₆) δ 10.51 (s, 1H), 8.10 (s, 1H), 7.85 (d, J = 8.2 Hz, 2H), 7.81 (s, 1H), 7.67-7.58 (m, 6H), 7.54 (t, J = 4.0 Hz, 2H), 4.40 (d, J = 18.4 Hz, 1H), 4.31 (d, J = 18.4 Hz, 1H), 2.44 (s, 3H). |
| 54 | 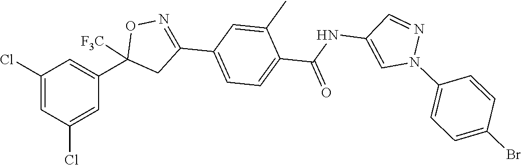 [SMILES: Cc1cc(C2=NOC(c3cc(Cl)cc(Cl)c3)(C(F)(F)F)C2)ccc1C(=O)Nc1cnn(-c2ccc(Br)cc2)c1] | ¹H NMR (600 MHz, DMSO-d₆) δ 10.51 (s, 1H), 8.18 (s, 1H), 7.81 (s, 1H), 7.67 (s, 2H), 7.65 (s, 2H), 7.59-7.56 (m, 3H), 7.55 (s, 1H), 7.23 (s, 1H), 7.22 (s, 1H), 4.41 (d, J = 18.4 Hz, 1H), 4.33 (d, J = 18.4 Hz, 1H), 2.42 (s, 3H). |
| 55 | 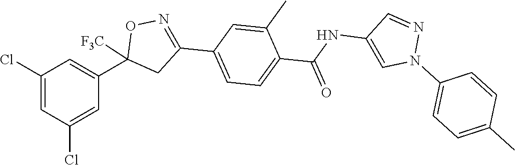 [SMILES: Cc1ccc(-n2cc(NC(=O)c3ccc(C4=NOC(c5cc(Cl)cc(Cl)c5)(C(F)(F)F)C4)cc3C)cn2)cc1] | ¹H NMR (600 MHz, DMSO-d₆) δ 10.48 (s, 1H), 8.10 (s, 1H), 7.81 (s, 1H), 7.67 (s, 2H), 7.65 (s, 2H), 7.56 (s, 1H), 7.55 (s, 1H), 7.17 (q, J = 8.0 Hz, 4H), 4.41 (d, J = 18.4 Hz, 1H), 4.32 (d, J = 18.4 Hz, 1H), 2.41 (s, 3H), 2.28 (s, 3H). |
| 56 | 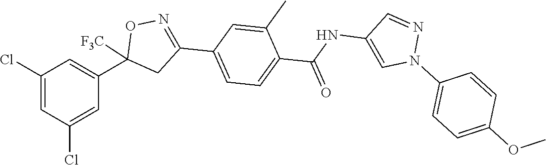 [SMILES: COc1ccc(-n2cc(NC(=O)c3ccc(C4=NOC(c5cc(Cl)cc(Cl)c5)(C(F)(F)F)C4)cc3C)cn2)cc1] | ¹H NMR (600 MHz, DMSO-d₆) δ 10.46 (s, 1H), 8.08 (d, J = 2.3 Hz, 1H), 7.82 (s, 1H), 7.66 (s, 1H), 7.64 (s, 3H), 7.55 (d, J = 8.1 Hz, 1H), 7.52 (s, 1H), 7.25 (d, J = 8.0 Hz, 2H), 6.92 (s, 1H), 6.91 (s, 1H), 4.40 (d, J = 18.4 Hz, 1H), 4.32 (d, J = 18.4 Hz, 1H), 3.74 (s, 3H), 2.40 (s, 3H). |
| 57 | 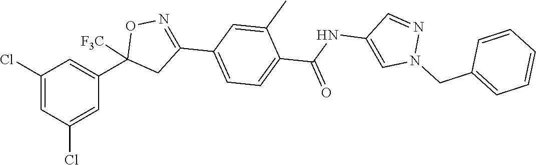 [SMILES: Cc1cc(C2=NOC(c3cc(Cl)cc(Cl)c3)(C(F)(F)F)C2)ccc1C(=O)Nc1cnn(Cc2ccccc2)c1] | ¹H NMR (600 MHz, DMSO-d₆) δ 10.49 (s, 1H), 8.13 (s, 1H), 7.83 (s, 1H), 7.69-7.60 (m, 4H), 7.55 (d, J = 8.9 Hz, 2H), 7.36 (t, J = 7.5 Hz, 2H), 7.30 (d, J = 7.2 Hz, 1H), 7.27 (d, J = 7.5 Hz, 2H), 5.31 (s, 2H), 4.40 (d, J = 18.4 Hz, 1H), 4.32 (d, J = 18.4 Hz, 1H), 2.40 (s, 3H). |
| 58 | 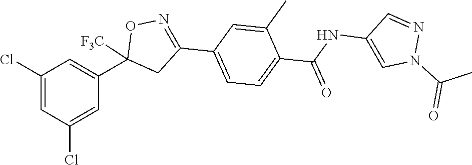 [SMILES: CC(=O)n1cc(NC(=O)c2ccc(C3=NOC(c4cc(Cl)cc(Cl)c4)(C(F)(F)F)C3)cc2C)cn1] | ¹H NMR (400 MHz, DMSO-d₆) δ 10.41 (s, 1H), 8.55 (s, 1H), 7.86 (d, J = 0.7 Hz, 1H), 7.82 (t, J = 1.9 Hz, 1H), 7.72 (d, J = 1.6 Hz, 1H), 7.70- 7.66 (m, 1H), 7.64 (d, J = 1.9 Hz, 2H), 7.60 (d, J = 8.0 Hz, 1H), 4.43 (d, J = 18.4 Hz, 1H), 4.34 (d, J = 18.4 Hz, 1H), 2.25 (s, 3H), 2.06 (s, 3H). |
| 59 | 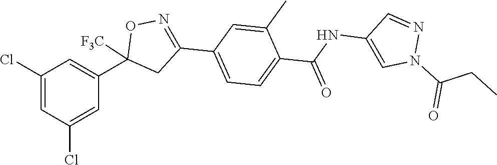 [SMILES: CCC(=O)n1cc(NC(=O)c2ccc(C3=NOC(c4cc(Cl)cc(Cl)c4)(C(F)(F)F)C3)cc2C)cn1] | ¹H NMR (400 MHz, DMSO-d₆) δ 10.33 (s, 1H), 8.56 (s, 1H), 7.87 (s, 1H), 7.83 (t, J = 1.9 Hz, 1H), 7.71 (s, 1H), 7.69-7.65 (m, 1H), 7.64 (d, J = 1.9 Hz, 2H), 7.60 (d, J = 8.0 Hz, 1H), 4.43 (d, J = 18.4 Hz, 1H), 4.34 (d, J = 18.4 Hz, 1H), 2.33 (q, J = 7.6 Hz, 2H), 2.25 (s, 3H), 1.09 (t, J = 7.6 Hz, 3H). |
| 60 | 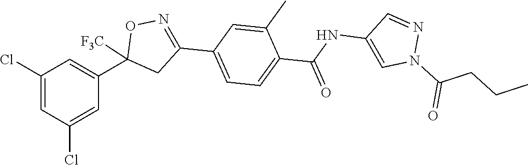 [SMILES: CCCC(=O)n1cc(NC(=O)c2ccc(C3=NOC(c4cc(Cl)cc(Cl)c4)(C(F)(F)F)C3)cc2C)cn1] | ¹H NMR (400 MHz, DMSO-d₆) δ 10.80 (s, 1H), 8.56 (s, 1H), 7.96 (d, J = 0.7 Hz, 1H), 7.82 (t, J = 1.9 Hz, 1H), 7.68 (d, J = 7.4 Hz, 2H), 7.64 (d, J = 1.8 Hz, 2H), 7.61 (d, J = 8.2 Hz, 1H), 4.41 (d, J = 18.4 Hz, 1H), 4.32 (d, J = 18.4 Hz, 1H), 3.07 (t, J = 7.3 Hz, 2H), 2.43 (s, 3H), 1.76- 1.64 (m, 2H), 0.96 (t, J = 7.4 Hz, 3H). |
| 61 | 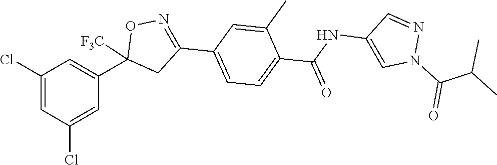 [SMILES: Cc1cc(C2=NOC(c3cc(Cl)cc(Cl)c3)(C(F)(F)F)C2)ccc1C(=O)Nc1cnn(C(=O)C(C)C)c1] | ¹H NMR (400 MHz, DMSO-d₆) δ 10.54 (s, 1H), 8.57 (s, 1H), 7.90 (s, 1H), 7.84 (t, J = 1.7 Hz, 1H), 7.78 (s, 1H), 7.72-7.67 (m, 1H), 7.63 (d, J = 1.7 Hz, 2H), 7.57 (d, J = 8.0 Hz, 1H), 4.43 (d, J = 18.4 Hz, 1H), 4.35 (d, J = 18.4 Hz, 1H), 3.54-3.38 (m, 1H), 2.45 (s, 3H), 1.34 (d, J = 6.7 Hz, 6H). |
| 62 | 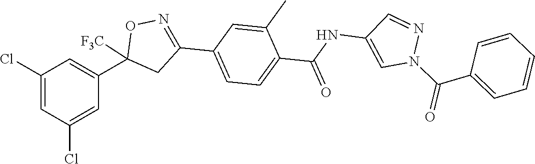 [SMILES: Cc1cc(C2=NOC(c3cc(Cl)cc(Cl)c3)(C(F)(F)F)C2)ccc1C(=O)Nc1cnn(C(=O)c2ccccc2)c1] | ¹H NMR (400 MHz, DMSO-d₆) δ 10.90 (s, 1H), 8.75 (s, 1H), 8.07 (s, 1H), 8.03 (d, J = 1.2 Hz, 1H), 8.01 (d, J = 1.6 Hz, 1H), 7.82 (t, J = 1.9 Hz, 1H), 7.72-7.67 (m, 3H), 7.67-7.64 (m, 3H), 7.58 (t, J = 7.7 Hz, 2H), 4.43 (d, J = 18.4 Hz, 1H), 4.34 (d, J = 18.4 Hz, 1H), 2.46 (s, 3H). |
| 63 | 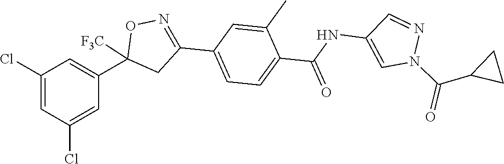 [SMILES: Cc1cc(C2=NOC(c3cc(Cl)cc(Cl)c3)(C(F)(F)F)C2)ccc1C(=O)Nc1cnn(C(=O)C2CC2)c1] | ¹H NMR (600 MHz, DMSO-d₆) δ 10.64 (s, 1H), 8.52 (s, 1H), 7.88 (s, 1H), 7.82 (s, 1H), 7.71 (s, 1H), 7.66 (d, J = 8.2 Hz, 1H), 7.63 (s, 2H), 7.60 (d, J = 8.0 Hz, 1H), 4.42 (d, J = 18.4 Hz, 1H), 4.33 (d, J = 18.4 Hz, 1H), 2.24 (s, 3H), 1.78-1.72 (m, 1H), 0.83 (d, J = 6.0 Hz, 4H). |
| 64 | 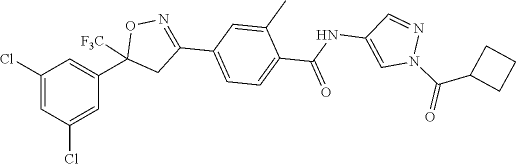 [SMILES: Cc1cc(C2=NOC(c3cc(Cl)cc(Cl)c3)(C(F)(F)F)C2)ccc1C(=O)Nc1cnn(C(=O)C2CCC2)c1] | ¹H NMR (600 MHz, DMSO-d₆) δ 10.53 (s, 1H), 8.57 (s, 1H), 7.83 (s, 1H), 7.73-7.60 (m, 4H), 7.56 (d, J = 7.8 Hz, 2H), 4.43 (d, J = 18.4 Hz, 1H), 4.36 (d, J = 18.4 Hz, 1H), 3.52-3.37 (m, 1H), 2.43 (s, 3H), 1.65-1.54 (m, 2H), 1.48-1.40 (m, 2H), 1.21-1.10 (m, 2H). |
| 65 | 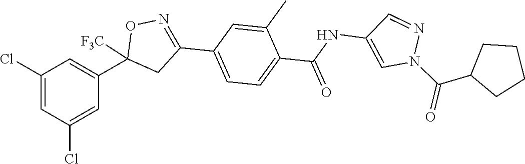 [SMILES: Cc1cc(C2=NOC(c3cc(Cl)cc(Cl)c3)(C(F)(F)F)C2)ccc1C(=O)Nc1cnn(C(=O)C2CCCC2)c1] | ¹H NMR (400 MHz, DMSO-d₆) δ 10.36 (s, 1H), 8.56 (s, 1H), 7.88 (s, 1H), 7.83 (t, J = 1.9 Hz, 1H), 7.71 (s, 1H), 7.67 (d, J = 8.0, 1.6 Hz, 1H), 7.64 (d, J = 1.9 Hz, 2H), 7.60 (d, J = 8.0 Hz, 1H), 4.43 (d, J = 18.4 Hz, 1H), 4.34 (d, J = 18.4 Hz, 1H), 2.76 (p, J = 7.9 Hz, 1H), 2.46 (s, 3H), 1.88-1.80 (m, 2H), 1.74-1.64 (m, 4H), 1.58-1.51 (m, 2H). |
| 66 | 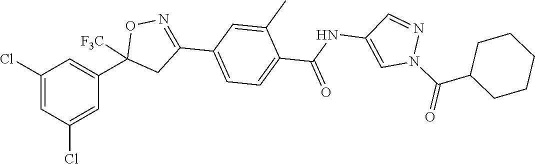 [SMILES: Cc1cc(C2=NOC(c3cc(Cl)cc(Cl)c3)(C(F)(F)F)C2)ccc1C(=O)Nc1cnn(C(=O)C2CCCCC2)c1] | ¹H NMR (600 MHz, DMSO-d₆) δ 10.44 (s, 1H), 8.41 (s, 1H), 7.97 (s, 1H), 7.82-7.77 (m, 4H), 7.68 (d, J = 7.8 Hz, 1H), 7.55 (s, 1H), 4.41 (d, J = 18.4 Hz, 1H), 4.34 (d, J = 18.4 Hz, 1H), 3.34-3.28 (m, 1H), 2.41 (s, 3H), 1.56-1.48 (m, 2H), 1.41 -1.37 (m, 2H), 1.28-1.24 (m, 2H), 1.14-1.10 (m, 2H), 0.95-0.88 (m, 2H). |
| 67 | 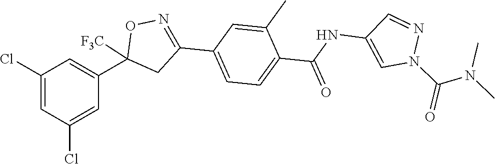 [SMILES: Cc1cc(C2=NOC(c3cc(Cl)cc(Cl)c3)(C(F)(F)F)C2)ccc1C(=O)Nc1cnn(C(=O)N(C)C)c1] | ¹H NMR (600 MHz, DMSO-d₆) δ 10.69 (s, 1H), 8.40 (s, 1H), 7.85 (s, 1H), 7.83 (s, 1H), 7.68 (s, 1H), 7.66 (s, 1H), 7.64 (s, 2H), 7.60 (d, J = 8.4 Hz, 1H), 4.41 (d, J = 18.4 Hz, 1H), 4.33 (d, J = 18.4 Hz, 1H), 3.13 (s, 6H), 2.43 (s, 3H). |
| 68 | 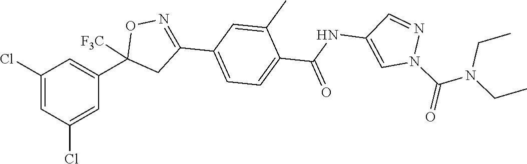 [SMILES: CCN(CC)C(=O)n1cc(NC(=O)c2ccc(C3=NOC(c4cc(Cl)cc(Cl)c4)(C(F)(F)F)C3)cc2C)cn1] | ¹H NMR (400 MHz, Chloroform-d) δ 8.45 (s, 1H), 7.85 (s, 1H), 7.72 (s, 1H), 7.54 (s, 2H), 7.51 (t, J = 2.6 Hz, 4H), 4.10 (d, J = 17.4 Hz, 1H), 3.72 (d, J = 17.4 Hz, 1H), 3.58 (q, J = 6.9Hz, 4H), 2.51 (s, 3H), 1.27 (t, J = 6.9 Hz, 6H). |
| 69 | 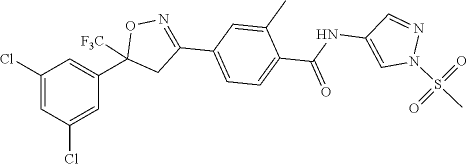 [SMILES: Cc1cc(C2=NOC(c3cc(Cl)cc(Cl)c3)(C(F)(F)F)C2)ccc1C(=O)Nc1cnn(S(C)(=O)=O)c1] | ¹H NMR (600 MHz, DMSO-d₆) δ 10.87 (s, 1H), 8.47 (s, 1H), 8.02 (s, 1H), 7.83 (t, J = 1.9 Hz, 1H), 7.68 (d, J = 9.6 Hz, 2H), 7.65-7.58 (m, 3H), 4.42 (d, J = 18.4 Hz, 1H), 4.33 (d, J = 18.4 Hz, 1H), 3.55 (s, 3H), 2.43 (s, 3H). |
| 70 | 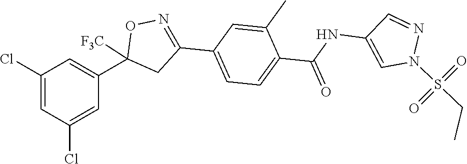 [SMILES: CCS(=O)(=O)n1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.86 (s, 1H), 8.47 (s, 1H), 8.04 (s, 1H), 7.83 (t, J = 1.9 Hz, 1H), 7.68 (d, J = 10.0 Hz, 2H), 7.66-7.60 (m, 3H), 4.42 (d, J = 18.4 Hz, 1H), 4.33 (d, J = 18.4 Hz, 1H), 3.71 (q, J = 7.3 Hz, 2H), 2.44 (s, 3H), 1.12 (t, J = 7.3 Hz, 3H). |
| 71 | 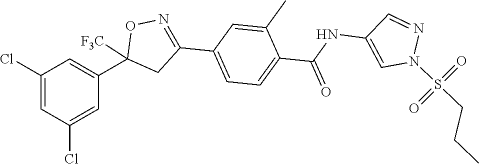 [SMILES: CCCS(=O)(=O)n1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.85 (s, 1H), 8.46 (s, 1H), 8.03 (s, 1H), 7.83 (s, 1H), 7.68 (d, J = 10.1 Hz, 2H), 7.65-7.60 (m, 3H), 4.41 (d, J = 18.4 Hz, 1H), 4.33 (d, J = 18.4 Hz, 1H), 3.68 (t, J = 7.6 Hz, 2H), 2.43 (s, 3H), 1.62-1.53 (m, 2H), 0.92 (t, J = 7.4 Hz, 3H). |
| 72 | 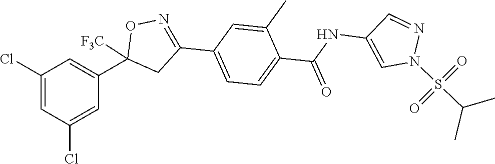 [SMILES: Cc1cc(C2=NOC(c3cc(Cl)cc(Cl)c3)(C(F)(F)F)C2)ccc1C(=O)Nc1cnn(S(=O)(=O)C(C)C)c1] | ¹H NMR (600 MHz, DMSO-d₆) δ 10.85 (s, 1H), 8.46 (s, 1H), 8.04 (s, 1H), 7.83 (s, 1H), 7.68 (d, J = 10.2 Hz, 2H), 7.65-7.61 (m, 3H), 4.41 (d, J = 18.4 Hz, 1H), 4.33 (d, J = 18.4 Hz, 1H), 3.94-3.86 (m, 1H), 2.44 (s, 3H), 1.24 (d, J = 6.8 Hz, 6H). |
| 73 | 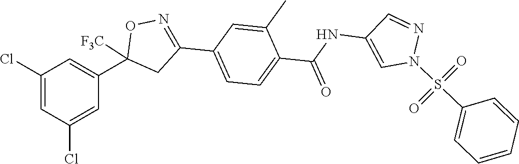 [SMILES: Cc1cc(C2=NOC(c3cc(Cl)cc(Cl)c3)(C(F)(F)F)C2)ccc1C(=O)Nc1cnn(S(=O)(=O)c2ccccc2)c1] | ¹H NMR (600 MHz, DMSO-d₆) δ 10.83 (s, 1H), 8.59 (s, 1H), 8.01 (d, J = 7.9 Hz, 2H), 7.96 (s, 1H), 7.84-7.79 (m, 2H), 7.71-7.64 (m, 4H), 7.63 (s, 2H), 7.59 (d, J = 7.9 Hz, 1H), 4.40 (d, J = 18.4 Hz, 1H), 4.32 (d, J = 18.4 Hz, 1H), 2.42 (s, 3H). |
| 74 | 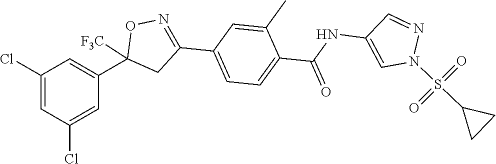 [SMILES: Cc1cc(C2=NOC(c3cc(Cl)cc(Cl)c3)(C(F)(F)F)C2)ccc1C(=O)Nc1cnn(S(=O)(=O)C2CC2)c1] | ¹H NMR (600 MHz, DMSO-d₆) δ 10.86 (s, 1H), 8.46 (s, 1H), 8.03 (s, 1H), 7.83 (d, J = 2.0 Hz, 1H), 7.68 (d, J = 10.2 Hz, 2H), 7.65-7.60 (m, 3H), 4.42 (d, J = 18.4 Hz, 1H), 4.33 (d, J = 18.4 Hz, 1H), 3.19- 3.12 (m, 1H), 2.44 (s, 3H), 1.29-1.25 (m, 2H), 1.25-1.22 (m, 2H). |
| 75 | 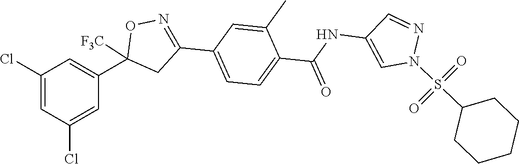 [SMILES: Cc1cc(C2=NOC(c3cc(Cl)cc(Cl)c3)(C(F)(F)F)C2)ccc1C(=O)Nc1cnn(S(=O)(=O)C2CCCCC2)c1] | ¹H NMR (600 MHz, DMSO-d₆) δ 10.72 (s, 1H), 8.30 (s, 1H), 7.89 (s, 1H), 7.65 (s, 1H), 7.53 (d, J = 8.3 Hz, 2H), 7.48 (d, J = 13.0 Hz, 3H), 4.26 (d, J = 18.4 Hz, 1H), 4.16 (d, J = 18.4 Hz, 1H), 3.58-3.52 (m, 1H), 2.29 (s, 3H), 1.72-1.66 (m, 2H), 1.63-1.58 (m, 2H), 1.46- 1.40 (m, 1H), 1.28-1.20 (m, 2H), 1.15-1.07 (m, 2H), 1.00-0.91 (m, 1H). |
| 76 | 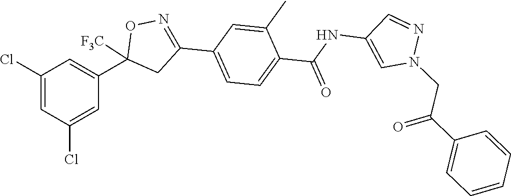 [SMILES: Cc1cc(C2=NOC(c3cc(Cl)cc(Cl)c3)(C(F)(F)F)C2)ccc1C(=O)Nc1cnn(CC(=O)c2ccccc2)c1] | ¹H NMR (600 MHz, DMSO-d₆) δ 10.49 (s, 1H), 8.33 (s, 1H), 8.17 (s, 1H), 7.95-7.87 (m, 4H), 7.74 (d, J = 8.9 Hz, 2H), 7.56 (t, J = 7.5 Hz, 2H), 7.47 (d, J = 7.2 Hz, 1H), 7.38 (d, J = 7.5 Hz, 2H), 5.81 (s, 2H), 4.40 (d, J = 18.4 Hz, 1H), 4.32 (d, J = 18.4 Hz, 1H), 2.40 (s, 3H). |
| 77 | 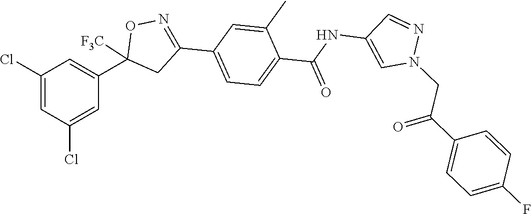 [SMILES: Cc1cc(C2=NOC(c3cc(Cl)cc(Cl)c3)(C(F)(F)F)C2)ccc1C(=O)Nc1cnn(CC(=O)c2ccc(F)cc2)c1] | ¹H NMR (600 MHz, DMSO-d₆) δ 10.51 (s, 1H), 8.16-8.11 (m, 2H), 8.10 (s, 1H), 7.83 (s, 1H), 7.67 (s, 1H), 7.66-7.63 (m, 3H), 7.60- 7.57 (m, 2H), 7.43 (t, J = 8.7 Hz, 2H), 5.82 (s, 2H), 4.41 (d, J = 18.4 Hz, 1H), 4.33 (d, J = 18.4 Hz, 1H), 2.43 (s, 3H). |
| 78 | 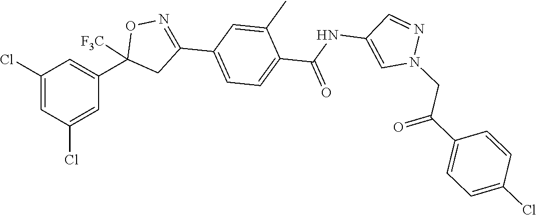 [SMILES: Cc1cc(C2=NOC(c3cc(Cl)cc(Cl)c3)(C(F)(F)F)C2)ccc1C(=O)Nc1cnn(CC(=O)c2ccc(Cl)cc2)c1] | ¹H NMR (600 MHz, DMSO-d₆) δ 10.51 (s, 1H), 8.10 (s, 1H), 8.05 (d, J = 8.2 Hz, 2H), 7.83 (s, 1H), 7.70-7.61 (m, 6H), 7.58 (t, J = 4.0 Hz, 2H), 5.82 (s, 2H), 4.41 (d, J = 18.4 Hz, 1H), 4.33 (d, J = 18.4 Hz, 1H), 2.43 (s, 3H). |
| 79 | 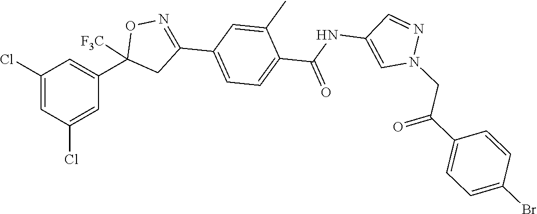 [SMILES: Cc1cc(C2=NOC(c3cc(Cl)cc(Cl)c3)(C(F)(F)F)C2)ccc1C(=O)Nc1cnn(CC(=O)c2ccc(Br)cc2)c1] | ¹H NMR (600 MHz, DMSO-d₆) δ 10.51 (s, 1H), 8.18 (s, 1H), 8.01 (s, 1H), 7.67 (s, 2H), 7.65 (s, 2H), 7.59-7.56 (m, 3H), 7.55 (s, 1H), 7.23 (s, 1H), 7.22 (s, 1H), 5.82 (s, 2H), 4.41 (d, J = 18.4 Hz, 1H), 4.33 (d, J = 18.4 Hz, 1H), 2.42 (s, 3H). |
| 80 | 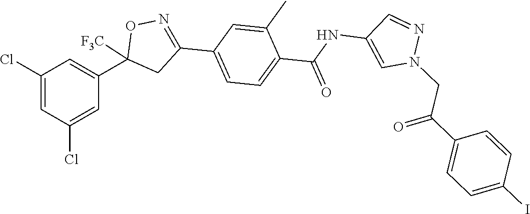 [SMILES: Cc1cc(C2=NOC(c3cc(Cl)cc(Cl)c3)(C(F)(F)F)C2)ccc1C(=O)Nc1cnn(CC(=O)c2ccc(I)cc2)c1] | ¹H NMR (600 MHz, DMSO-d₆) δ 10.54 (s, 1H), 8.24 (s, 1H), 8.13 8.10 (m, 1H), 7.91 (d, J = 3.8 Hz, 1H), 7.81 (t, J = 12.7 Hz, 5H), 7.71 (d, J = 7.3 Hz, 1H), 7.61-7.56 (m, 1H), 7.31-7.26 (m, 1H), 7.05 (d, J = 7.5 Hz, 1H), 5.85 (s, 2H), 4.42 (d, J = 18.4 Hz, 1H), 4.33 (d, J = 18.4 Hz, 1H), 2.43 (s, 3H). |
| 81 | 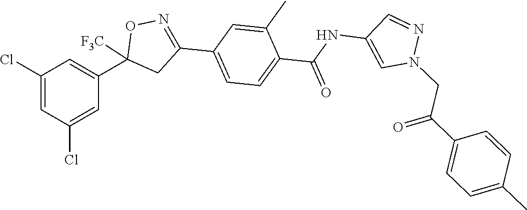 [SMILES: Cc1ccc(C(=O)Cn2cc(NC(=O)c3ccc(C4=NOC(c5cc(Cl)cc(Cl)c5)(C(F)(F)F)C4)cc3C)cn2)cc1] | ¹H NMR (600 MHz, DMSO-d₆) δ 10.48 (s, 1H), 8.30 (s, 1H), 8.11 (s, 1H), 7.67 (s, 2H), 7.65 (s, 2H), 7.56 (s, 1H), 7.55 (s, 1H), 7.25-7.11 (m, 4H), 5.76 (s, 2H), 4.41 (d, J = 18.4 Hz, 1H), 4.32 (d, J = 18.4 Hz, 1H), 2.41 (s, 3H), 2.28 (s, 3H). |
| 82 | 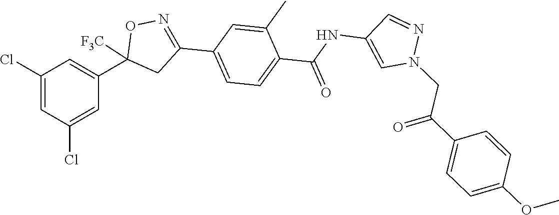 [SMILES: COc1ccc(C(=O)Cn2cc(NC(=O)c3ccc(C4=NOC(c5cc(Cl)cc(Cl)c5)(C(F)(F)F)C4)cc3C)cn2)cc1] | ¹H NMR (600 MHz, DMSO-d₆) δ 10.46 (s, 1H), 8.18 (d, J = 2.3 Hz, 1H), 8.02 (s, 1H), 7.66 (s, 1H), 7.64 (s, 3H), 7.55 (d, J = 8.1 Hz, 1H), 7.52 (s, 1H), 7.25 (d, J = 8.0 Hz, 2H), 6.94 (s, 1H), 6.91 (s, 1H), 5.73 (s, 2H), 4.40 (d, J = 18.4 Hz, 1H), 4.32 (d, J = 18.4 Hz, 1H), 3.74 (s, 3H), 2.40 (s, 3H). |
| 83 | 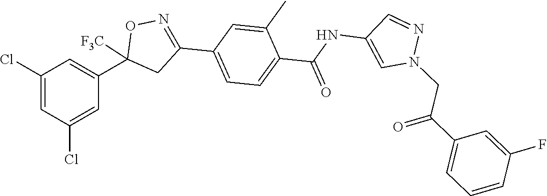 [SMILES: Cc1cc(C2=NOC(c3cc(Cl)cc(Cl)c3)(C(F)(F)F)C2)ccc1C(=O)Nc1cnn(CC(=O)c2cccc(F)c2)c1] | ¹H NMR (600 MHz, DMSO-d₆) δ 10.51 (s, 1H), 8.31 (s, 1H), 8.15 (s, 1H), 8.11 (s, 1H), 7.85 (s, 2H), 7.71 (d, J = 6.2 Hz, 2H), 7.42 (d, J = 7.5 Hz, 1H), 7.37 (s, 1H), 7.30 (t, J = 11.7 Hz, 3H), 5.86 (s, 2H), 4.42 (d, J = 18.4 Hz, 1H), 4.34 (d, J = 18.4 Hz, 1H), 2.42 (s, 3H). |
| 84 | 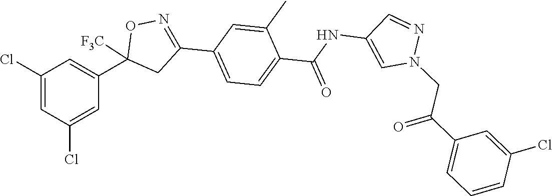 [SMILES: Cc1cc(C2=NOC(c3cc(Cl)cc(Cl)c3)(C(F)(F)F)C2)ccc1C(=O)Nc1cnn(CC(=O)c2cccc(Cl)c2)c1] | ¹H NMR (400 MHz, DMSO-d₆) δ 10.50 (s, 1H), 8.32 (s, 1H), 8.17 (t, J = 1.9 Hz, 1H), 7.93-7.83 (m, 4H), 7.70-7.63 (m, 2H), 7.53-7.44 (m, 2H), 7.39 (d, J = 2.3 Hz, 1H), 7.34-7.25 (m, 1H), 5.84 (s, 2H), 4.41 (d, J = 18.4 Hz, 1H), 4.32 (d, J = 18.4 Hz, 1H), 2.41 (s, 3H). |
| 85 | 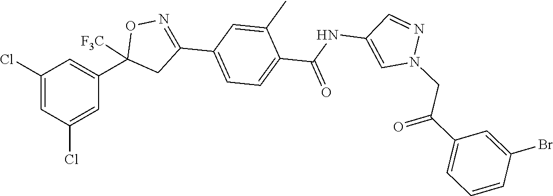 [SMILES: Cc1cc(C2=NOC(c3cc(Cl)cc(Cl)c3)(C(F)(F)F)C2)ccc1C(=O)Nc1cnn(CC(=O)c2cccc(Br)c2)c1] | ¹H NMR (600 MHz, DMSO-d₆) δ 10.52 (s, 1H), 8.34-8.28 (m, 1H), 8.21 (d, J = 9.9 Hz, 1H), 7.97 (s, 1H), 7.81 (s, 3H), 7.61-7.76 (m, 2H), 7.64 (d, J = 7.9 Hz, 1H), 7.58 (s, 1H), 7.51 (t, J = 7.6 Hz, 1H), 7.45 (d, J = 7.7 Hz, 1H), 5.84 (s, 2H), 4.41 (d, J = 18.4 Hz, 1H), 4.33 (d, J = 18.4 Hz, 1H), 2.42 (s, 3H). |
| 86 | 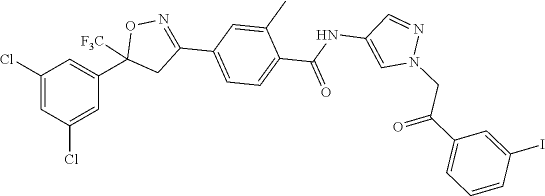 [SMILES: Cc1cc(C2=NOC(c3cc(Cl)cc(Cl)c3)(C(F)(F)F)C2)ccc1C(=O)Nc1cnn(CC(=O)c2cccc(I)c2)c1] | ¹H NMR (600 MHz, DMSO-d₆) δ 10.52 (s, 1H), 8.25 (s, 1H), 8.17- 8.14 (m, 1H), 7.97-7.85 (m, 4H), 7.74-7.67 (m, 2H), 7.62-7.47 (m, 2H), 7.41 (d, J = 2.3 Hz, 1H), 7.28 (s, 1H), 5.81 (s, 2H), 4.42 (d, J = 18.4 Hz, 1H), 4.32 (d, J = 18.4 Hz, 1H), 2.42 (s, 3H). |
| 87 | 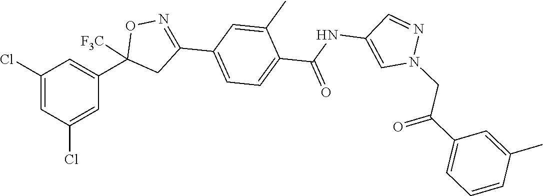 [SMILES: Cc1cccc(C(=O)Cn2cc(NC(=O)c3ccc(C4=NOC(c5cc(Cl)cc(Cl)c5)(C(F)(F)F)C4)cc3C)cn2)c1] | ¹H NMR (600 MHz, DMSO-d₆) δ 10.52 (s, 1H), 8.26 (s, 1H), 8.13 (s, 1H), 7.95 (s, 1H), 7.84 (s, 3H), 7.70 (d, J = 4.3 Hz, 1H), 7.67 (s, 1H), 7.43 (d, J = 7.7 Hz, 1H), 7.35 (d, J = 8.1 Hz, 2H), 7.28 (d, J = 8.0 Hz, 1H), 5.79 (s, 2H), 4.43 (d, J = 18.3 Hz, 1H), 4.34 (d, J = 18.3 Hz, 1H), 3.43 (s, 3H), 2.29 (s, 3H). |
| 88 | 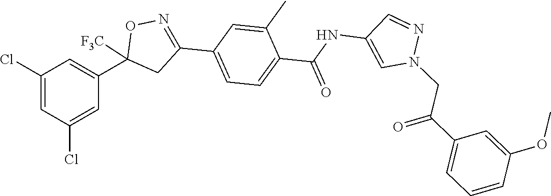 [SMILES: COc1cccc(C(=O)Cn2cc(NC(=O)c3ccc(C4=NOC(c5cc(Cl)cc(Cl)c5)(C(F)(F)F)C4)cc3C)cn2)c1] | ¹H NMR (600 MHz, DMSO-d₆) δ 10.47 (s, 1H), 8.33 (d, J = 2.6 Hz, 1H), 8.19-8.14 (m, 1H), 7.95 (d, J = 13.1 Hz, 4H), 7.83-7.75 (m, 2H), 7.64 (t, J = 7.9 Hz, 1H), 7.41 (s, 2H), 7.24 (d, J = 7.4 Hz, 1H), 5.76 (s, 2H), 4.38 (d, J = 18.4 Hz, 1H), 4.30 (d, J = 18.4 Hz, 1H), 3.71 (s, 3H), 2.40 (s, 3H). |
| 89 | 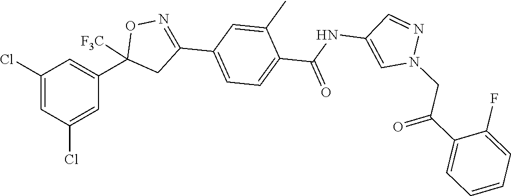 [SMILES: Cc1cc(C2=NOC(c3cc(Cl)cc(Cl)c3)(C(F)(F)F)C2)ccc1C(=O)Nc1cnn(CC(=O)c2ccccc2F)c1] | ¹H NMR (600 MHz, DMSO-d₆) δ 10.47 (s, 1H), 8.26 (s, 1H), 8.17 (s, 1H), 8.02 (d, J = 12.7 Hz, 4H), 7.85 (d, J = 6.9 Hz, 2H), 7.68-7.62 (m, 1H), 7.59-7.46 (m, 3H), 5.88 (s, 2H), 4.41 (d, J = 18.4 Hz, 1H), 4.32 (d, J = 18.4 Hz, 1H), 2.44 (s, 3H). |
| 90 | 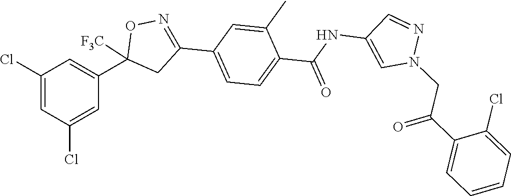 [SMILES: Cc1cc(C2=NOC(c3cc(Cl)cc(Cl)c3)(C(F)(F)F)C2)ccc1C(=O)Nc1cnn(CC(=O)c2ccccc2Cl)c1] | ¹H NMR (400 MHz, DMSO-d₆) δ 10.52 (s, 1H), 8.35 (s, 1H), 8.26 (s, 1H), 8.18-8.01 (m, 4H), 7.91-7.84 (m, 2H), 7.74 (s, 1H), 7.56- 7.48 (m, 2H), 7.36-7.29 (m, 1H), 5.93 (s, 2H), 4.40 (d, J = 18.4 Hz, 1H), 4.34 (d, J = 18.4 Hz, 1H), 2.45 (s, 3H). |
| 91 | 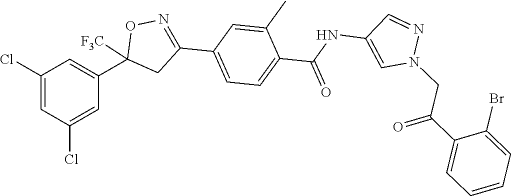 [SMILES: Cc1cc(C2=NOC(c3cc(Cl)cc(Cl)c3)(C(F)(F)F)C2)ccc1C(=O)Nc1cnn(CC(=O)c2ccccc2Br)c1] | ¹H NMR (600 MHz, DMSO-d₆) δ 10.49 (s, 1H), 8.31 (s, 1H), 8.25 (s, 1H), 8.11-8.03 (m, 6H), 7.94 (d, J = 7.9 Hz, 1H), 7.79 (t, J = 7.4 Hz, 1H), 7.68 (t, J = 7.7 Hz, 1H), 7.42 (d, J = 7.7 Hz, 1H), 5.91 (s, 2H), 4.42 (d, J = 18.4 Hz, 1H), 4.35 (d, J = 18.4 Hz, 1H), 2.41 (s, 3H). |
| 92 | 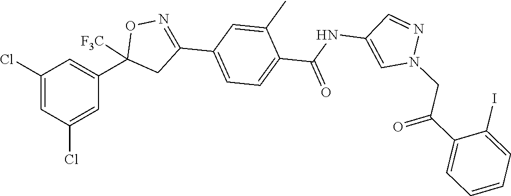 [SMILES: Cc1cc(C2=NOC(c3cc(Cl)cc(Cl)c3)(C(F)(F)F)C2)ccc1C(=O)Nc1cnn(CC(=O)c2ccccc2I)c1] | ¹H NMR (600 MHz, DMSO-d₆) δ 10.54 (s, 1H), 8.35 (s, 1H), 8.23 8.18 (m, 1H), 8.04 (d, J = 3.8 Hz, 1H), 7.85 (t, J = 12.7 Hz, 5H), 7.79 (d, J = 7.3 Hz, 1H), 7.61- 7.55 (m, 1H), 7.41 - 7.36 (m, 1H), 7.22 (d, J = 7.5 Hz, 1H), 5.85 (s, 2H), 4.45 (d, J = 18.3 Hz, 1H), 4.32 (d, J = 18.3 Hz, 1H), 2.41 (s, 3H). |
| 93 | 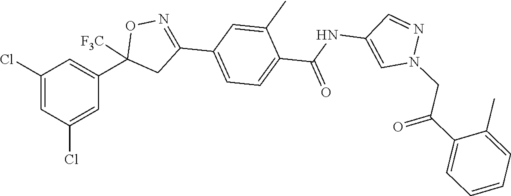 [SMILES: Cc1ccccc1C(=O)Cn1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.45 (s, 1H), 8.28 (s, 1H), 8.13 8.08 (m, 1H), 7.89-7.81 (m, 4H), 7.62 (d, J = 8.1 Hz, 2H), 7.41- 7.31 (m, 3H), 7.27 (d, J = 7.5 Hz, 1H), 5.82 (s, 2H), 4.39 (d, J = 18.4 Hz, 1H), 4.32 (d, J = 18.4 Hz, 1H), 2.42 (s, 3H), 2.33 (s, 3H). |
| 94 | 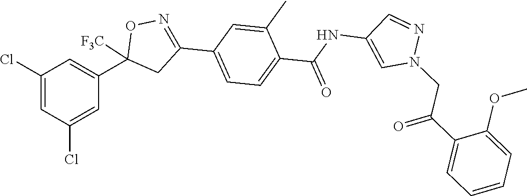 [SMILES: COc1ccccc1C(=O)Cn1cc(NC(=O)c2ccc(C3=NOC(c4cc(Cl)cc(Cl)c4)(C(F)(F)F)C3)cc2C)cn1] | ¹H NMR (600 MHz, DMSO-d₆) δ 10.45 (s, 1H), 8.23 (d, J = 2.8 Hz, 1H), 8.21-8.11 (m, 1H), 7.88 (d, J = 13.5 Hz, 4H), 7.78-7.70 (m, 2H), 7.63 (t, J =8.0 Hz, 1H), 7.50 (s, 2H), 7.33 (d, J = 7.6 Hz, 1H), 5.79 (s, 2H), 4.37 (d, J = 18.4 Hz, 1H), 4.30 (d, J = 18.4 Hz, 1H), 3.72 (s, 3H), 2.42 (s, 3H). |
| 95 | 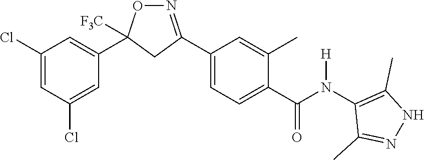 [SMILES: Cc1cc(C2=NOC(c3cc(Cl)cc(Cl)c3)(C(F)(F)F)C2)ccc1C(=O)Nc1c(C)n[nH]c1C] | ¹H NMR (600 MHz, DMSO-d₆) δ 12.16 (s, 1H), 9.43 (s, 1H), 7.83 (t, J = 1.9 Hz, 1H), 7.67-7.63 (m, 4H), 7.56 (d, J = 7.8 Hz, 1H), 4.40 (d, J = 18.4 Hz, 1H), 4.33 (d, J = 18.4 Hz, 1H), 2.45 (s, 3H), 2.09 (s, 6H). |
| 96 | 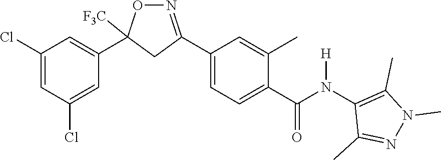 [SMILES: Cc1cc(C2=NOC(c3cc(Cl)cc(Cl)c3)(C(F)(F)F)C2)ccc1C(=O)Nc1c(C)nn(C)c1C] | ¹H NMR (600 MHz, DMSO-d₆) δ 9.47 (s, 1H), 7.82 (d, J = 13.0 Hz, 1H), 7.65 (d, J = 10.3 Hz, 4H), 7.57 (d, J = 7.4 Hz, 1H), 4.40 (d, J = 18.4 Hz, 1H), 4.32 (d, J = 18.4 Hz, 1H), 3.65 (s, 3H), 2.44 (s, 3H), 2.12 (s, 3H), 2.03 (s, 3H). |
| 97 | 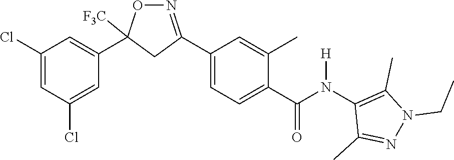 [SMILES: CCn1nc(C)c(NC(=O)c2ccc(C3=NOC(c4cc(Cl)cc(Cl)c4)(C(F)(F)F)C3)cc2C)c1C] | ¹H NMR (600 MHz, DMSO-d₆) δ 9.46 (s, 1H), 7.83 (s, 1H), 7.66 (d, J = 11.1 Hz, 4H), 7.57 (d, J = 7.8 Hz, 1H), 4.41 (d, J = 18.4 Hz, 1H), 4.33 (d, J = 18.4 Hz, 1H), 3.98 (q, J = 7.2 Hz, 2H), 2.45 (s, 3H), 2.14 (s, 3H), 2.05 (s, 3H), 1.28 (t, J = 7.2 Hz, 3H). |
| 98 | 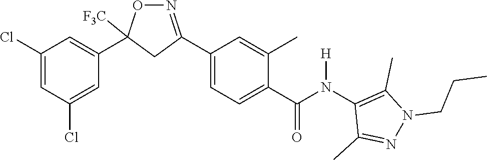 [SMILES: CCCn1nc(C)c(NC(=O)c2ccc(C3=NOC(c4cc(Cl)cc(Cl)c4)(C(F)(F)F)C3)cc2C)c1C] | ¹H NMR (600 MHz, DMSO-d₆) δ 9.47 (s, 1H), 7.83 (t, J = 1.9 Hz, 1H), 7.66 (d, J = 11.2 Hz, 4H), 7.58 (d, J = 7.8 Hz, 1H), 4.41 (d, J = 18.4 Hz, 1H), 4.33 (d, J = 18.4 Hz, 1H), 3.90 (t, J = 7.2 Hz, 2H), 2.45 (s, 3H), 2.14 (s, 3H), 2.06 (s, 3H), 1.78-1.64 (m, 2H), 0.86 (t, J = 7.4 Hz, 3H). |
| 99 | 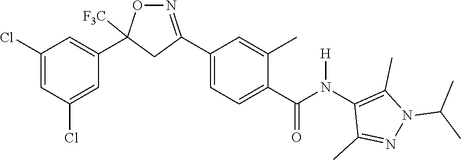 [SMILES: Cc1cc(C2=NOC(c3cc(Cl)cc(Cl)c3)(C(F)(F)F)C2)ccc1C(=O)Nc1c(C)nn(C(C)C)c1C] | ¹H NMR (600 MHz, DMSO-d₆) δ 9.44 (s, 1H), 7.83 (s, 1H), 7.66 (d, J = 11.0 Hz, 4H), 7.57 (d, J = 7.9 Hz, 1H), 4.46-4.42 (m, 1H), 4.41 (d, J = 18.4 Hz, 1H), 4.33 (d, J = 18.4 Hz, 1H), 2.45 (s, 3H), 2.15 (s, 3H), 2.06 (s, 3H), 1.35 (d, J = 6.6 Hz, 6H). |
| 100 | 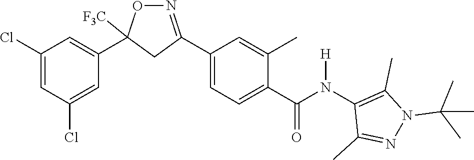 [SMILES: Cc1cc(C2=NOC(c3cc(Cl)cc(Cl)c3)(C(F)(F)F)C2)ccc1C(=O)Nc1c(C)nn(C(C)(C)C)c1C] | ¹H NMR (600 MHz, DMSO-d₆) δ 9.41 (s, 1H), 7.83 (s, 1H), 7.65 (d, J = 11.1 Hz, 4H), 7.57 (d, J = 7.9 Hz, 1H), 4.40 (d, J = 18.4 Hz, 1H), 4.32 (d, J = 18.4 Hz, 1H), 2.45 (s, 3H), 2.28 (s, 3H), 2.04 (s, 3H), 1.55 (s, 9H). |
| 101 | 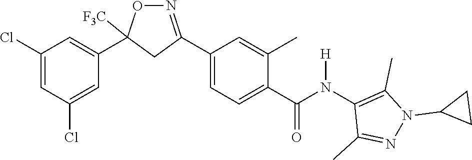 [SMILES: Cc1cc(C2=NOC(c3cc(Cl)cc(Cl)c3)(C(F)(F)F)C2)ccc1C(=O)Nc1c(C)nn(C2CC2)c1C] | ¹H NMR (400 MHz, Chloroform-d) δ 7.56 (d, J = 2.2 Hz, 3H), 7.52 (d, J = 1.8 Hz, 2H), 7.44 (t, J = 1.9 Hz, 1H), 7.00 (s, 1H), 4.10 (d, J = 17.4 Hz, 1H), 3.72 (d, J = 17.4 Hz, 1H), 3.33-3.25 (m, 1H), 2.54 (s, 3H), 2.31 (s, 3H), 2.20 (s, 3H), 1.20 - 1.14 (m, 2H), 1.09-1.02 (m, 2H). |
| 102 | 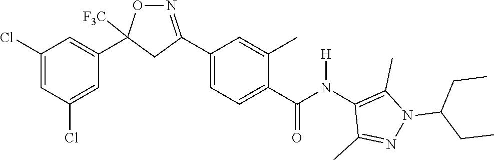 [SMILES: CCC(CC)n1nc(C)c(NC(=O)c2ccc(C3=NOC(c4cc(Cl)cc(Cl)c4)(C(F)(F)F)C3)cc2C)c1C] | ¹H NMR (600 MHz, DMSO-d₆) δ 9.44 (s, 1H), 7.83 (s, 1H), 7.69- 7.52 (m, 5H), 4.41 (d, J = 18.4 Hz, 1H), 4.33 (d, J = 18.4 Hz, 1H), 3.93- 3.80 (m, 1H), 2.45 (s, 3H), 2.14 (s, 3H), 2.08 (s, 3H), 1.85-1.75 (m, 2H), 1.75-1.63 (m, 2H), 0.68 (t, J = 7.2 Hz, 6H). |
| 103 | 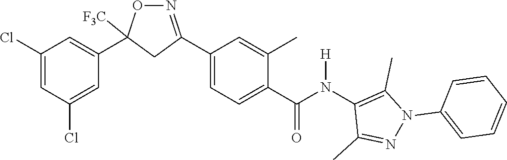 [SMILES: Cc1cc(C2=NOC(c3cc(Cl)cc(Cl)c3)(C(F)(F)F)C2)ccc1C(=O)Nc1c(C)nn(-c2ccccc2)c1C] | ¹H NMR (400 MHz, DMSO-d₆) δ 9.68 (s, 1H), 7.83 (t, J = 1.9 Hz, 1H), 7.71-7.59 (m, 5H), 7.53 (d, J = 4.3 Hz, 4H), 7.45-7.37 (m, 1H), 4.42 (d, J = 18.4 Hz, 1H), 4.33 (d, J = 18.4 Hz, 1H), 2.48 (s, 3H), 2.25 (s, 3H), 2.17 (s, 3H). |
| 104 | 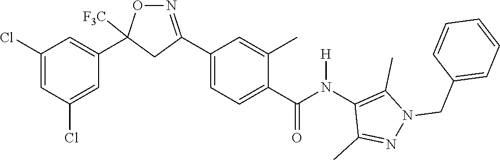 [SMILES: Cc1cc(C2=NOC(c3cc(Cl)cc(Cl)c3)(C(F)(F)F)C2)ccc1C(=O)Nc1c(C)nn(Cc2ccccc2)c1C] | ¹H NMR (400 MHz, Chloroform-d) δ 7.55 (s, 3H), 7.52 (d, J = 1.9 Hz, 2H), 7.44 (t, J = 1.9 Hz, 1H), 7.32 (d, J = 1.8 Hz, 1H), 7.31-7.26 (m, 2H), 7.16-7.11 (m, 2H), 6.95 (s, 1H), 5.23 (s, 2H), 4.10 (d, J = 17.4 Hz, 1H), 3.72 (d, J = 17.4 Hz, 1H), 2.54 (s, 3H), 2.23 (s, 3H), 2.14 (s, 3H). |
| 105 | 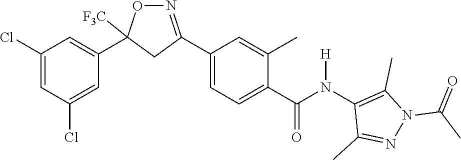 [SMILES: CC(=O)n1nc(C)c(NC(=O)c2ccc(C3=NOC(c4cc(Cl)cc(Cl)c4)(C(F)(F)F)C3)cc2C)c1C] | ¹H NMR (600 MHz, DMSO-d₆) δ 9.85 (s, 1H), 7.83 (s, 1H), 7.68 (d, J = 11.0 Hz, 2H), 7.65 (s, 3H), 4.42 (d, J = 18.4 Hz, 1H), 4.34 (d, J = 18.4 Hz, 1H), 2.60 (s, 3H), 2.51 (s, 3H), 2.41 (s, 3H), 2.18 (s, 3H). |
| 106 | 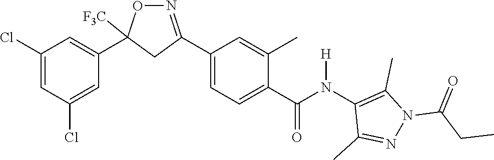 [SMILES: CCC(=O)n1nc(C)c(NC(=O)c2ccc(C3=NOC(c4cc(Cl)cc(Cl)c4)(C(F)(F)F)C3)cc2C)c1C] | ¹H NMR (400 MHz, DMSO-d₆) δ 9.83 (s, 1H), 7.85 (s, 1H), 7.73-7.65 (m, 5H) 4.43 (d, J = 18.4 Hz, 1H), 4.35 (d, J = 18.4 Hz, 1H), 3.15 (q, J = 7.6 Hz, 2H), 2.52 (s, 3H), 2.25 (s, 3H), 2.18 (s, 3H), 1.59 (t, J = 7.6 Hz, 3H). |
| 107 | 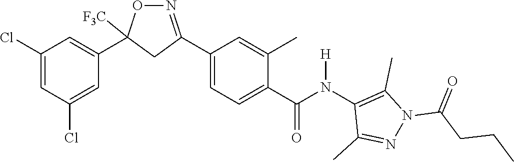 [SMILES: CCCC(=O)n1nc(C)c(NC(=O)c2ccc(C3=NOC(c4cc(Cl)cc(Cl)c4)(C(F)(F)F)C3)cc2C)c1C] | ¹H NMR (400 MHz, DMSO-d₆) δ 9.84 (s, 1H), 7.85 (t, J = 1.9 Hz, 1H), 7.71-7.63 (m, 5H), 4.43 (d, J = 18.4 Hz, 1H), 4.34 (d, J = 18.4 Hz, 1H), 3.07 (t, J = 7.3 Hz, 2H), 2.47 (s, 3H), 2.43 (s, 3H), 2.19 (s, 3H), 1.75-1.63 (m, 2H), 0.98 (t, J = 7.4 Hz, 3H). |
| 108 | 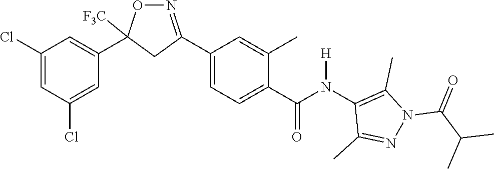 [SMILES: Cc1cc(C2=NOC(c3cc(Cl)cc(Cl)c3)(C(F)(F)F)C2)ccc1C(=O)Nc1c(C)nn(C(=O)C(C)C)c1C] | ¹H NMR (400 MHz, DMSO-d₆) δ 9.84 (s, 1H), 7.82 (s, 1H), 7.78-7.72 (m, 5H) 4.44 (d, J = 18.4 Hz, 1H), 4.35 (d, J = 18.4 Hz, 1H), 3.48- 3.42 (m, 1H), 2.48 (s, 3H), 2.40 (s, 3H), 2.17 (s, 3H), 1.37 (d, J = 6.7 Hz, 6H). |
| 109 | 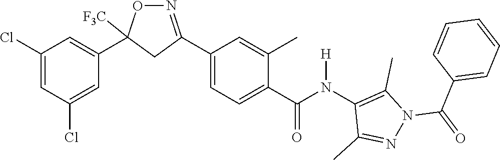 [SMILES: Cc1cc(C2=NOC(c3cc(Cl)cc(Cl)c3)(C(F)(F)F)C2)ccc1C(=O)Nc1c(C)nn(C(=O)c2ccccc2)c1C] | ¹H NMR (400 MHz, DMSO-d₆) δ 10.90 (s, 1H), 8.52 (s, 1H), 8.04 (s, 1H), 8.01 (d, J = 1.2 Hz, 1H), 7.86 (d, J = 1.6 Hz, 1H), 7.82 (t, J = 1.9 Hz, 1H), 7.77-7.68 (m, 2H), 7.63-7.60 (m, 2H), 7.49 (t, J = 7.6 Hz, 2H), 4.43 (d, J = 18.4 Hz, 1H), 4.34 (d, J = 18.4 Hz, 1H), 2.48 (s, 3H), 2.41 (s, 3H), 2.19 (s, 3H). |
| 110 | 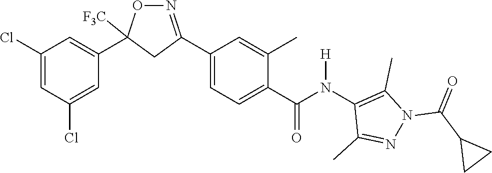 [SMILES: Cc1cc(C2=NOC(c3cc(Cl)cc(Cl)c3)(C(F)(F)F)C2)ccc1C(=O)Nc1c(C)nn(C(=O)C2CC2)c1C] | ¹H NMR (600 MHz, DMSO-d₆) δ 9.86 (s, 1H), 7.83 (d, J = 2.7 Hz, 1H), 7.69 (s, 1H), 7.67 (s, 1H), 7.65 (s, 3H), 4.42 (d, J = 18.4 Hz, 1H), 4.33 (d, J = 18.4 Hz, 1H), 3.16-3.10 (m, 1H), 2.46 (s, 3H), 2.40 (s, 3H), 2.21 (s, 3H), 1.13-1.09 (m, 2H), 1.08-1.04 (m, 2H). |
| 111 | 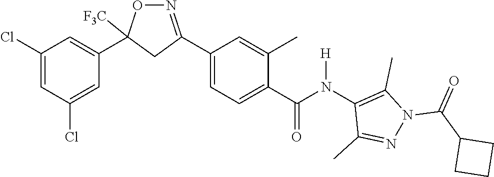 [SMILES: Cc1cc(C2=NOC(c3cc(Cl)cc(Cl)c3)(C(F)(F)F)C2)ccc1C(=O)Nc1c(C)nn(C(=O)C2CCC2)c1C] | ¹H NMR (600 MHz, DMSO-d₆) δ 9.81 (s, 1H), 7.82 (s, 1H), 7.67 (d, J = 9.9 Hz, 2H), 7.64 (d, J = 6.1 Hz, 3H), 4.41 (d, J = 18.4 Hz, 1H), 4.33 (d, J = 18.4 Hz, 1H), 4.26-4.19 (m, 1H), 2.45 (s, 3H), 2.42 (s, 3H), 2.32-2.27 (m, 2H), 2.25-2.21 (m, 2H), 2.16 (s, 3H), 2.06- 1.98 (m, 2H). |
| 112 | 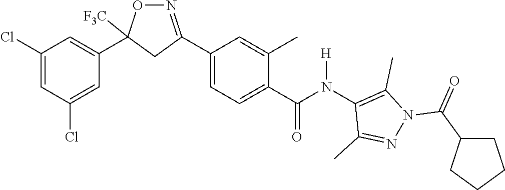 [SMILES: Cc1cc(C2=NOC(c3cc(Cl)cc(Cl)c3)(C(F)(F)F)C2)ccc1C(=O)Nc1c(C)nn(C(=O)C2CCCC2)c1C] | ¹H NMR (600 MHz, DMSO-d₆) δ 9.81 (s, 1H), 7.81 (d, J = 2.5 Hz, 1H), 7.65 (d, J = 9.9 Hz, 2H), 7.62 (s, 3H), 4.38 (d, J = 18.4 Hz, 1H), 4.31 (d, J = 18.4 Hz, 1H), 3.98-3.91 (m, 1H), 2.43 (s, 3H), 2.39 (s, 3H), 2.15 (s, 3H), 1.79-1.71 (m, 2H), 1.71-1.64 (m, 2H), 1.63- 1.56 (m, 2H), 0.86- 0.78 (m, 2H). |
| 113 | 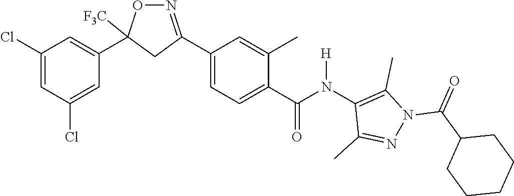 [SMILES: Cc1cc(C2=NOC(c3cc(Cl)cc(Cl)c3)(C(F)(F)F)C2)ccc1C(=O)Nc1c(C)nn(C(=O)C2CCCCC2)c1C] | ¹H NMR (600 MHz, DMSO-d₆) δ 9.84 (s, 1H), 7.83 (s, 1H), 7.68 (d, J = 9.8 Hz, 2H), 7.66-7.61 (m, 3H), 4.42 (d, J = 18.4 Hz, 1H), 4.33 (d, J = 18.4 Hz, 1H), 3.68-3.58 (m, 1H), 2.46 (s, 3H), 2.40 (s, 3H), 2.19 (s, 3H), 2.11-2.06 (m, 1H), 1.93-1.86 (m, 2H), 1.81-1.72 (m, 2H), 1.71-1.62 (m, 1H), 1.48-1.40 (m, 2H), 1.40-1.31 (m, 2H). |
| 114 | 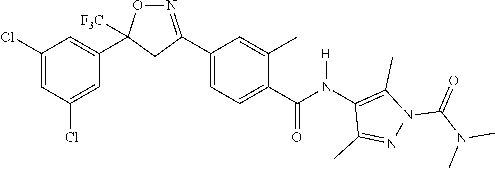 [SMILES: Cc1cc(C2=NOC(c3cc(Cl)cc(Cl)c3)(C(F)(F)F)C2)ccc1C(=O)Nc1c(C)nn(C(=O)N(C)C)c1C] | ¹H NMR (600 MHz, DMSO-d₆) δ 9.68 (s, 1H), 7.84-7.80 (m, 1H), 7.68 (d, J = 10.6 Hz, 2H), 7.65 (s, 2H), 7.61 (d, J = 7.9 Hz, 1H), 4.41 (d, J = 18.4 Hz, 1H), 4.33 (d, J = 18.4 Hz, 1H), 3.03 (s, 6H), 2.46 (s, 3H), 2.25 (s, 3H), 2.13 (s, 3H). |
| 115 | 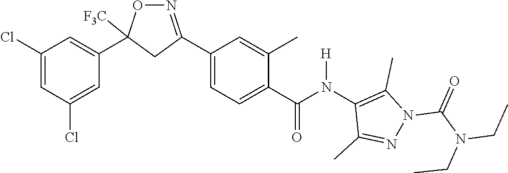 [SMILES: CCN(CC)C(=O)n1nc(C)c(NC(=O)c2ccc(C3=NOC(c4cc(Cl)cc(Cl)c4)(C(F)(F)F)C3)cc2C)c1C] | ¹H NMR (400 MHz, DMSO-d₆) δ 9.68 (s, 1H), 7.83 (t, J = 1.9 Hz, 1H), 7.68 (s, 1H), 7.65 (d, J = 5.7, 3H), 7.61 (d, J = 7.9 Hz, 1H), 4.39 (d, J = 18.4 Hz, 1H), 4.33 (d, J = 18.4 Hz, 1H), 3.39 (q, J = 7.5 Hz, 4H), 2.46 (s, 3H), 2.23 (s, 3H), 2.13 (s, 3H), 1.16 (t, J = 7.5 Hz, 6H). |
| 116 | 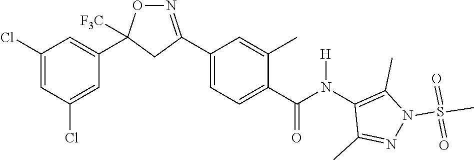 [SMILES: Cc1cc(C2=NOC(c3cc(Cl)cc(Cl)c3)(C(F)(F)F)C2)ccc1C(=O)Nc1c(C)nn(S(C)(=O)=O)c1C] | ¹H NMR (400 MHz, Chloroform-d) δ 7.58 (s, 3H), 7.52 (d, J = 1.9 Hz, 2H), 7.44 (t, J = 1.9 Hz, 1H), 7.10 (s, 1H), 4.11 (d, J = 17.4 Hz, 1H), 3.73 (d, J = 17.4 Hz, 1H), 3.32 (s, 3H), 2.54 (s, 3H), 2.44 (s, 3H), 2.26 (s, 3H). |
| 117 | 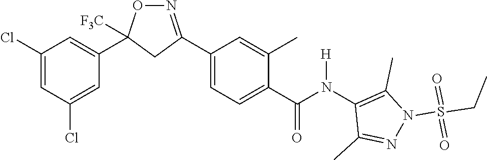 [SMILES: CCS(=O)(=O)n1nc(C)c(NC(=O)c2ccc(C3=NOC(c4cc(Cl)cc(Cl)c4)(C(F)(F)F)C3)cc2C)c1C] | ¹H NMR (600 MHz, DMSO-d₆) δ 9.86 (s, 1H), 7.83 (t, J = 2.3 Hz, 1H), 7.68 (d, J = 10.8 Hz, 2H), 7.66-7.63 (m, 3H), 4.42 (d, J = 18.4 Hz, 1H), 4.34 (d, J = 18.4 Hz, 1H), 3.65 (q, J = 7.4 Hz, 2H), 2.46 (s, 3H), 2.37 (s, 3H), 2.18 (s, 3H), 1.13 (t, J = 7.4 Hz, 3H). |
| 118 | 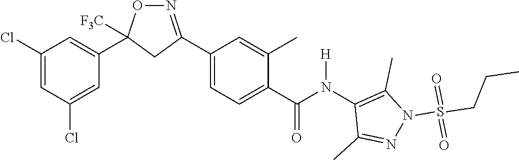 [SMILES: CCCS(=O)(=O)n1nc(C)c(NC(=O)c2ccc(C3=NOC(c4cc(Cl)cc(Cl)c4)(C(F)(F)F)C3)cc2C)c1C] | ¹H NMR (600 MHz, DMSO-d₆) δ 9.84 (s, 1H), 7.83 (s, 1H), 7.67 (d, J = 11.0 Hz, 2H), 7.64 (s, 3H), 4.41 (d, J = 18.4 Hz, 1H), 4.33 (d, J = 18.4 Hz, 1H), 3.62 (t, J = 8.7 Hz, 2H), 2.46 (s, 3H), 2.36 (s, 3H), 2.17 (s, 3H), 1.62-1.53 (m, 2H), 0.93 (t, J = 6.5, 3H). |
| 119 | 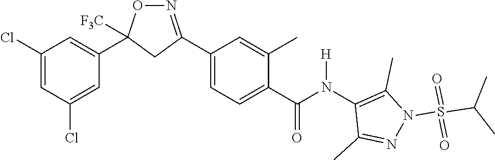 [SMILES: Cc1cc(C2=NOC(c3cc(Cl)cc(Cl)c3)(C(F)(F)F)C2)ccc1C(=O)Nc1c(C)nn(S(=O)(=O)C(C)C)c1C] | ¹H NMR (600 MHz, DMSO-d₆) δ 9.85 (s, 1H), 7.84 (s, 1H), 7.68 (d, J = 11.9 Hz, 2H), 7.64 (s, 3H), 4.42 (d, J = 18.4 Hz, 1H), 4.33 (d, J = 18.4 Hz, 1H), 3.82 (m, 1H), 2.46 (s, 3H), 2.36 (s, 3H), 2.18 (s, 3H), 1.22 (d, J = 6.6 Hz, 6H). |
| 120 | 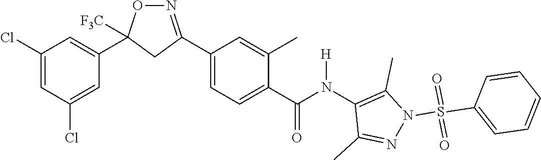 [SMILES: Cc1cc(C2=NOC(c3cc(Cl)cc(Cl)c3)(C(F)(F)F)C2)ccc1C(=O)Nc1c(C)nn(S(=O)(=O)c2ccccc2)c1C] | ¹H NMR (600 MHz, DMSO-d₆) δ 9.80 (s, 1H), 7.98 (d, J = 7.4 Hz, 2H), 7.83 (s, 1H), 7.79 (d, J = 7.9 Hz, 1H), 7.69 (d, J = 7.6 Hz, 1H), 7.66 (s, 2H), 7.63 (s, 3H), 7.60 (d, J = 7.8 Hz, 1H), 4.40 (d, J = 18.4 Hz, 1H), 4.32 (d, J = 18.4 Hz, 1H), 2.42 (d, J = 2.8 Hz, 6H), 2.11 (s, 3H). |
| 121 | 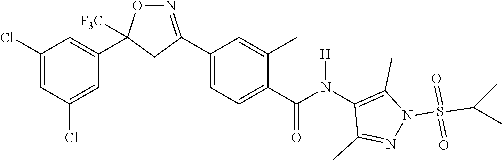 [SMILES: Cc1cc(C2=NOC(c3cc(Cl)cc(Cl)c3)(C(F)(F)F)C2)ccc1C(=O)Nc1c(C)nn(S(=O)(=O)C(C)C)c1C] | ¹H NMR (600 MHz, DMSO-d₆) δ 9.81 (s, 1H), 7.80 (s, 1H), 7.65 (d, J = 10.6 Hz, 2H), 7.62 (d, J = 2.3 Hz, 3H), 4.39 (d, J = 18.4 Hz, 1H), 4.30 (d, J = 18.4 Hz, 1H), 3.17-3.12 (m, 1H), 2.44 (s, 3H), 2.34 (s, 3H), 2.15 (s, 3H), 1.22-1.20 (m, 2H), 1.16-1.13 (m, 2H). |
| 122 | 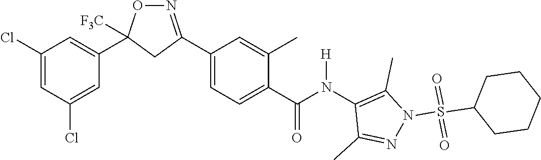 [SMILES: Cc1cc(C2=NOC(c3cc(Cl)cc(Cl)c3)(C(F)(F)F)C2)ccc1C(=O)Nc1c(C)nn(S(=O)(=O)C2CCCCC2)c1C] | ¹H NMR (600 MHz, DMSO-d₆) δ 9.83 (s, 1H), 7.83 (d, J = 2.0 Hz, 1H), 7.68 (d, J = 11.4 Hz, 2H), 7.65 (d, J = 4.6 Hz, 3H), 4.41 (d, J = 18.4 Hz, 1H), 4.33 (d, J = 18.4 Hz, 1H), 3.67-3.61 (m, 1H), 2.46 (s, 3H), 2.36 (s, 3H), 2.18 (s, 3H), 2.18-2.11 (m, 1H), 2.14-2.08 (m, 1H), 1.82-1.76 (m, 4H), 1.76-1.72 (m, 4H). |
| 123 | 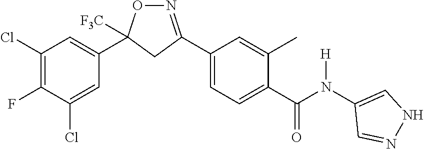 [SMILES: Cc1cc(C2=NOC(c3cc(Cl)c(F)c(Cl)c3)(C(F)(F)F)C2)ccc1C(=O)Nc1cn[nH]c1] | ¹H NMR (600 MHz, DMSO-d₆) δ 12.69 (s, 1H), 10.44 (s, 1H), 7.82 (d, J = 5.9 Hz, 3H), 7.64 (d, J = 10.2 Hz, 3H), 7.56 (d, J = 7.9 Hz, 1H), 4.40 (d, J = 18.4 Hz, 1H), 4.34 (d, J = 18.4 Hz, 1H), 2.42 (s, 3H). |
| 124 | 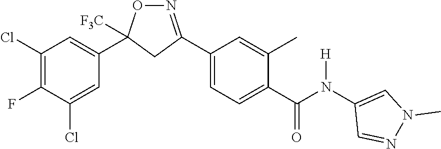 [SMILES: Cc1cc(C2=NOC(c3cc(Cl)c(F)c(Cl)c3)(C(F)(F)F)C2)ccc1C(=O)Nc1cnn(C)c1] | ¹H NMR (600 MHz, DMSO-d₆) δ 10.44 (s, 1H), 8.01 (s, 1H), 7.82 (d, J = 5.9 Hz, 2H), 7.64 (d, J = 10.3 Hz, 2H), 7.55 (d, J = 7.9 Hz, 1H), 7.49 (s, 1H), 4.40 (d, J = 18.4 Hz, 1H), 4.34 (d, J = 18.4 Hz, 1H), 3.82 (s, 3H), 2.41 (s, 3H). |
| 125 | 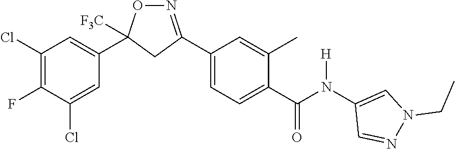 [SMILES: CCn1cc(NC(=O)c2ccc(C3=NOC(c4cc(Cl)c(F)c(Cl)c4)(C(F)(F)F)C3)cc2C)cn1] | ¹H NMR (600 MHz, DMSO-d₆) δ 10.44 (s, 1H), 8.04 (s, 1H), 7.82 (d, J = 6.1 Hz, 2H), 7.64 (d, J = 10.2 Hz, 2H), 7.55 (d, J = 7.9 Hz, 1H), 7.50 (s, 1H), 4.39 (d, J = 18.4 Hz, 1H), 4.34 (d, J = 18.4 Hz, 1H), 4.11 (q, J = 7.3 Hz, 2H), 2.41 (s, 3H), 1.36 (t, J = 7.3 Hz, 3H). |
| 126 | 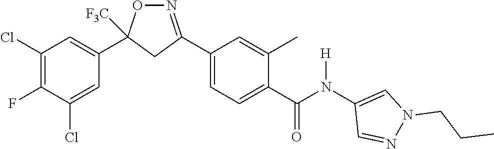 [SMILES: CCCn1cc(NC(=O)c2ccc(C3=NOC(c4cc(Cl)c(F)c(Cl)c4)(C(F)(F)F)C3)cc2C)cn1] | ¹H NMR (600 MHz, DMSO-d₆) δ 10.44 (s, 1H), 8.03 (s, 1H), 7.82 (d, J = 6.0 Hz, 2H), 7.64 (d, J = 10.7 Hz, 2H), 7.56 (d, J = 7.9 Hz, 1H), 7.51 (s, 1H), 4.40 (d, J = 18.4 Hz, 1H), 4.34 (d, J = 18.4 Hz, 1H), 4.04 (t, J = 7.0 Hz, 2H), 2.42 (s, 3H), 1.83-1.72 (m, 2H), 0.83 (t, J = 7.3 Hz, 3H). |
| 127 | 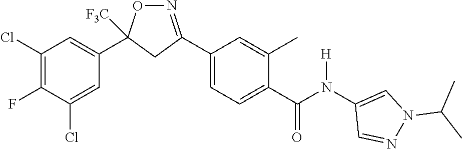 [SMILES: Cc1cc(C2=NOC(c3cc(Cl)c(F)c(Cl)c3)(C(F)(F)F)C2)ccc1C(=O)Nc1cnn(C(C)C)c1] | ¹H NMR (600 MHz, DMSO-d₆) δ 10.44 (s, 1H), 8.04 (s, 1H), 7.83 (d, J = 6.0 Hz, 2H), 7.65 (d, J = 10.0 Hz, 2H), 7.56 (d, J = 7.8 Hz, 1H), 7.52 (s, 1H), 4.54 - 4.45 (m, 1H), 4.40 (d, J = 18.4 Hz, 1H), 4.34 (d, J = 18.4 Hz, 1H), 2.42 (s, 3H), 1.41 (d, J = 6.6 Hz, 6H). |
| 128 | 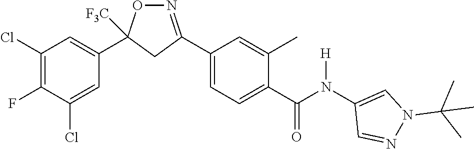 [SMILES: Cc1cc(C2=NOC(c3cc(Cl)c(F)c(Cl)c3)(C(F)(F)F)C2)ccc1C(=O)Nc1cnn(C(C)(C)C)c1] | ¹H NMR (600 MHz, DMSO-d₆) δ 10.42 (s, 1H), 8.06 (s, 1H), 7.82 (d, J = 6.0 Hz, 2H), 7.64 (d, J = 10.2 Hz, 2H), 7.54 (d, J = 9.0 Hz, 2H), 4.39 (d, J = 18.4 Hz, 1H), 4.33 (d, J = 18.4 Hz, 1H), 2.41 (s, 3H), 1.52 (s, 9H). |
| 129 | 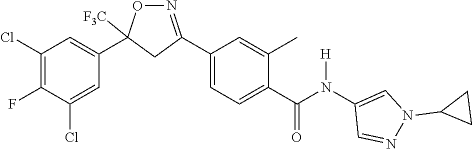 [SMILES: Cc1cc(C2=NOC(c3cc(Cl)c(F)c(Cl)c3)(C(F)(F)F)C2)ccc1C(=O)Nc1cnn(C2CC2)c1] | ¹H NMR (600 MHz, DMSO-d₆) δ 10.42 (s, 1H), 8.00 (s, 1H), 7.79 (d, J = 6.1 Hz, 2H), 7.61 (d, J = 10.3 Hz, 2H), 7.52 (d, J = 8.0 Hz, 1H), 7.45 (s, 1H), 4.37 (d, J = 18.4 Hz, 1H), 4.31 (d, J = 18.4 Hz, 1H), 3.73- 3.64 (m, 1H), 2.38 (s, 3H), 1.01-0.97 (m, 2H), 0.94-0.89 (m, 2H). |
| 130 | 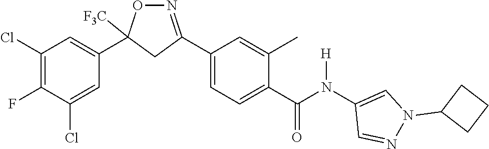 [SMILES: Cc1cc(C2=NOC(c3cc(Cl)c(F)c(Cl)c3)(C(F)(F)F)C2)ccc1C(=O)Nc1cnn(C2CCC2)c1] | ¹H NMR (600 MHz, DMSO-d6) 8 10.46 (s, 1H), 8.08 (s, 1H), 7.82 (d, J = 6.0 Hz, 2H), 7.64 (d, J = 9.6 Hz, 2H), 7.55 (d, J = 9.0 Hz, 2H), 4.86-4.78 (m, 1H), 4.40 (d, J = 18.4 Hz, 1H), 4.34 (d, J = 18.4 Hz, 1H), 2.48-2.42 (m, 2H), 2.41 (s, 3H), 2.39-2.31 (m, 2H), 1.82 1.71 (m, 2H). |
| 131 | 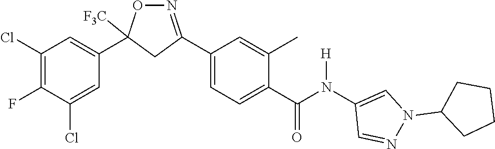 [SMILES: Cc1cc(C2=NOC(c3cc(Cl)c(F)c(Cl)c3)(C(F)(F)F)C2)ccc1C(=O)Nc1cnn(C2CCCC2)c1] | ¹H NMR (600 MHz, DMSO-d₆) δ 10.43 (s, 1H), 8.03 (s, 1H), 7.82 (d, J = 6.1 Hz, 2H), 7.64 (d, J = 10.0 Hz, 2H), 7.55 (d, J = 7.8 Hz, 1H), 7.51 (s, 1H), 4.72-4.65 (m, 1H), 4.39 (d, J = 18.4 Hz, 1H), 4.34 (d, J = 18.4 Hz, 1H), 2.41 (s, 3H), 2.10-2.01 (m, 2H), 1.95-1.84 (m, 2H), 1.84-1.72 (m, 2H), 1.69-1.58 (m, 2H). |
| 132 | 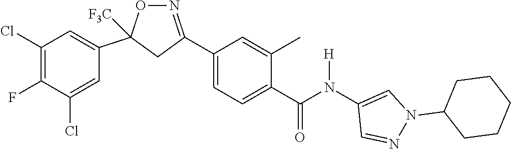 [SMILES: Cc1cc(C2=NOC(c3cc(Cl)c(F)c(Cl)c3)(C(F)(F)F)C2)ccc1C(=O)Nc1cnn(C2CCCCC2)c1] | ¹H NMR (600 MHz, DMSO-d₆) δ 10.44 (s, 1H), 8.01 (s, 1H), 7.82 (d, J = 6.0 Hz, 2H), 7.64 (d, J = 10.4 Hz, 2H), 7.55 (d, J = 7.9 Hz, 1H), 7.51 (s, 1H), 4.39 (d, J = 18.4 Hz, 1H), 4.34 (d, J = 18.4 Hz, 1H), 4.15- 4.08 (m, 1H), 2.41 (s, 3H), 2.00-1.95 (m, 2H), 1.84-1.76 (m, 2H), 1.75-1.61 (m, 2H), 1.46-1.33 (m, 2H), 1.29-1.15 (m, 2H). |
| 133 | 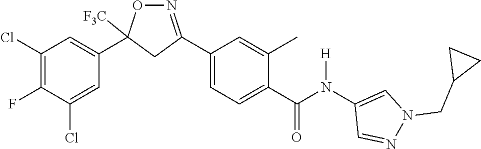 [SMILES: Cc1cc(C2=NOC(c3cc(Cl)c(F)c(Cl)c3)(C(F)(F)F)C2)ccc1C(=O)Nc1cnn(CC2CC2)c1] | ¹H NMR (600 MHz, DMSO-d₆) δ 10.45 (s, 1H), 8.09 (s, 1H), 7.83 (d, J = 6.0 Hz, 2H), 7.65 (d, J = 10.3 Hz, 2H), 7.56 (d, J = 7.9 Hz, 1H), 7.51 (s, 1H), 4.40 (d, J = 18.4 Hz, 1H), 4.34 (d, J = 18.4 Hz, 1H), 3.95 (d, J = 7.1 Hz, 2H), 2.42 (s, 3H), 1.28-1.20 (m, 1H), 0.57-0.49 (m, 2H), 0.40-0.33 (m, 2H). |
| 134 | 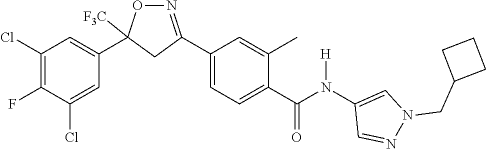 [SMILES: Cc1cc(C2=NOC(c3cc(Cl)c(F)c(Cl)c3)(C(F)(F)F)C2)ccc1C(=O)Nc1cnn(CC2CCC2)c1] | ¹H NMR (600 MHz, DMSO-d₆) δ 10.42 (s, 1H), 7.98 (s, 1H), 7.80 (d, J = 6.0 Hz, 2H), 7.64-7.58 (m, 2H), 7.53 (d, J = 7.9 Hz, 1H), 7.47 (s, 1H), 4.37 (d, J = 18.4 Hz, 1H), 4.31 (d, J = 18.4 Hz, 1H), 4.08 (d, J = 7.2 Hz, 2H), 2.74-2.66 (m, 1H), 2.39 (s, 3H), 1.96-1.90 (m, 2H), 1.85-1.77 (m, 2H), 1.77-1.69 (m, 2H). |
| 135 | 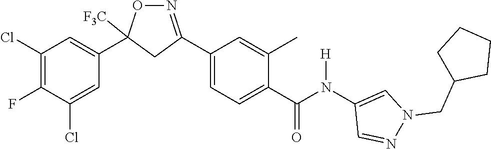 [SMILES: Cc1cc(C2=NOC(c3cc(Cl)c(F)c(Cl)c3)(C(F)(F)F)C2)ccc1C(=O)Nc1cnn(CC2CCCC2)c1] | ¹H NMR (600 MHz, DMSO-d₆) δ 10.44 (s, 1H), 8.03 (s, 1H), 7.82 (d, J = 6.0 Hz, 2H), 7.64 (d, J = 10.9 Hz, 2H), 7.56 (d, J = 7.9 Hz, 1H), 7.50 (s, 1H), 4.40 (d, J = 18.4 Hz, 1H), 4.34 (d, J = 18.4 Hz, 1H), 4.00 (d, J = 7.4 Hz, 2H), 2.41 (s, 3H), 2.38-2.31 (m, 1H), 1.65-1.55 (m, 4H), 1.54-1.44 (m, 2H), 1.28-1.20 (m, 2H). |
| 136 | 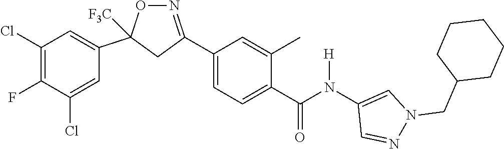 [SMILES: Cc1cc(C2=NOC(c3cc(Cl)c(F)c(Cl)c3)(C(F)(F)F)C2)ccc1C(=O)Nc1cnn(CC2CCCCC2)c1] | ¹H NMR (600 MHz, DMSO-d₆) δ 10.44 (s, 1H), 7.99 (s, 1H), 7.82 (d, J = 6.0 Hz, 2H), 7.64 (d, J = 10.7 Hz, 2H), 7.56 (d, J = 7.9 Hz, 1H), 7.51 (s, 1H), 4.40 (d, J = 18.4 Hz, 1H), 4.34 (d, J = 18.4 Hz, 1H), 4.07- 3.98 (m, 1H), 3.92 (d, J = 7.1 Hz, 2H), 2.42 (s, 3H), 1.83-1.57 (m, 4H), 1.56-1.46 (m, 2H), 1.20-1.17 (m, 2H), 1.00-0.88 (m, 2H). |
| 137 | 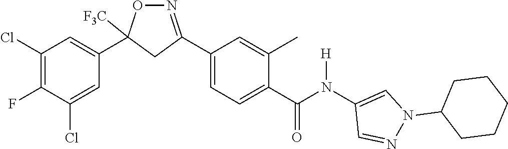 [SMILES: Cc1cc(C2=NOC(c3cc(Cl)c(F)c(Cl)c3)(C(F)(F)F)C2)ccc1C(=O)Nc1cnn(C2CCCCC2)c1] | ¹H NMR (600 MHz, DMSO-d₆) δ 10.43 (s, 1H), 8.01 (s, 1H), 7.82 (d, J = 6.1 Hz, 2H), 7.67-7.61 (m, 2H), 7.58 (d, J = 7.9 Hz, 1H), 7.54 (s, 1H), 4.39 (d, J = 18.4 Hz, 1H), 4.34 (d, J = 18.4 Hz, 1H), 4.00-3.92 (m, 1H), 2.42 (s, 3H), 1.84-1.69 (m, 4H), 0.70 (t, J = 7.4 Hz, 6H). |
| 138 | 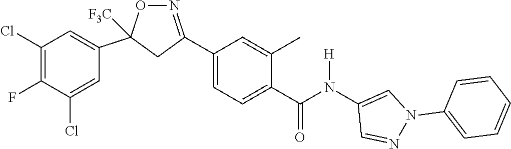 [SMILES: Cc1cc(C2=NOC(c3cc(Cl)c(F)c(Cl)c3)(C(F)(F)F)C2)ccc1C(=O)Nc1cnn(-c2ccccc2)c1] | ¹H NMR (600 MHz, DMSO-d₆) δ 10.66 (s, 1H), 8.65 (s, 1H), 7.84 - 7.77 (m, 5H), 7.65 (d, J = 9.1 Hz, 2H), 7.60 (d, J = 7.8 Hz, 1H), 7.48 (t, J = 7.9 Hz, 2H), 7.28 (t, J = 7.5 Hz, 1H), 4.38 (d, J = 18.4 Hz, 1H), 4.33 (d, J = 18.4 Hz, 1H), 2.43 (s, 3H). |
| 139 | 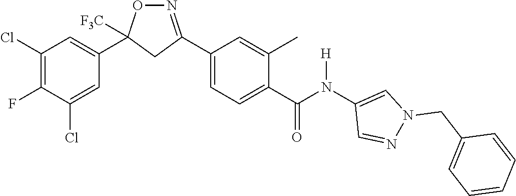 [SMILES: Cc1cc(C2=NOC(c3cc(Cl)c(F)c(Cl)c3)(C(F)(F)F)C2)ccc1C(=O)Nc1cnn(Cc2ccccc2)c1] | ¹H NMR (600 MHz, DMSO-d₆) δ 10.48 (s, 1H), 8.14 (s, 1H), 7.82 (d, J = 6.0 Hz, 2H), 7.65 (d, J = 10.1 Hz, 2H), 7.57 (d, J = 7.3 Hz, 2H), 7.36 (t, J = 7.4 Hz, 2H), 7.31 (d, J = 7.3 Hz, 1H), 7.27 (d, J = 7.5 Hz, 2H), 5.32 (s, 2H), 4.40 (d, J = 18.4 Hz, 1H), 4.34 (d, J = 18.4 Hz, 1H), 2.42 (s, 3H). |
| 140 | 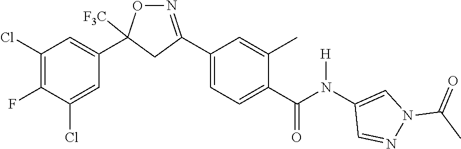 [SMILES: CC(=O)n1cc(NC(=O)c2ccc(C3=NOC(c4cc(Cl)c(F)c(Cl)c4)(C(F)(F)F)C3)cc2C)cn1] | ¹H NMR (400 MHz, DMSO-d₆) δ 10.42 (s, 1H), 8.55 (s, 1H), 7.85 (s, 1H), 7.82 (d, J = 6.1 Hz, 2H), 7.70 (s, 1H), 7.66 (d, J = 8.3 Hz, 1H), 7.61 (d, J = 8.1 Hz, 1H), 4.43 (d, J = 18.4 Hz, 1H), 4.35 (d, J = 18.4 Hz, 1H), 2.25 (s, 3H), 2.06 (s, 3H). |
| 141 | 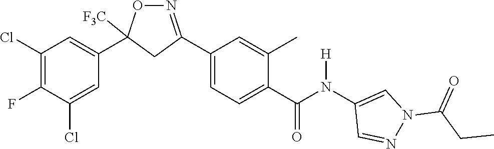 [SMILES: CCC(=O)n1cc(NC(=O)c2ccc(C3=NOC(c4cc(Cl)c(F)c(Cl)c4)(C(F)(F)F)C3)cc2C)cn1] | ¹H NMR (600 MHz, DMSO-d₆) δ 10.34 (s, 1H), 8.56 (s, 1H), 7.87 (s, 1H), 7.82 (d, J = 6.0 Hz, 2H), 7.70 (s, 1H), 7.66 (d, J = 8.0 Hz, 1H), 7.61 (d, J = 8.1 Hz, 1H), 4.42 (d, J = 18.4 Hz, 1H), 4.35 (d, J = 18.4 Hz, 1H), 2.33 (q, J = 7.6 Hz, 2H), 2.25 (s, 3H), 1.09 (t, J = 7.6 Hz, 3H). |
| 142 | 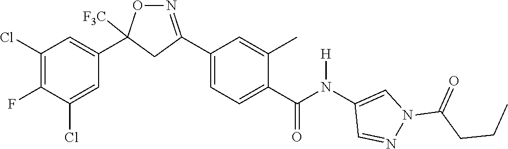 [SMILES: CCCC(=O)n1cc(NC(=O)c2ccc(C3=NOC(c4cc(Cl)c(F)c(Cl)c4)(C(F)(F)F)C3)cc2C)cn1] | ¹H NMR (600 MHz, DMSO-d₆) δ 10.37 (s, 1H), 8.57 (s, 1H), 7.85 (s, 1H), 7.81 (d, J = 6.2 Hz, 2H), 7.68 (s, 1H), 7.65 (d, J = 8.2 Hz, 1H), 7.63 (d, J = 8.0 Hz, 1H), 4.41 (d, J = 18.4 Hz, 1H), 4.35 (d, J = 18.4 Hz, 1H), 3.07 (t, J = 7.3 Hz, 2H), 2.43 (s, 3H), 1.76 - 1.64 (m, 2H), 0.96 (t, J = 7.4 Hz, 3H). |
| 143 | 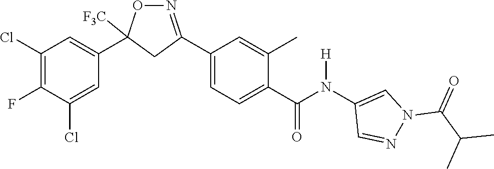 [SMILES: Cc1cc(C2=NOC(c3cc(Cl)c(F)c(Cl)c3)(C(F)(F)F)C2)ccc1C(=O)Nc1cnn(C(=O)C(C)C)c1] | ¹H NMR (600 MHz, DMSO-d₆) δ 10.35 (s, 1H), 8.55 (s, 1H), 7.87 (s, 1H), 7.83 (d, J = 6.1 Hz, 2H), 7.66 (s, 1H), 7.63 (d, J = 7.9 Hz, 1H), 7.61 (d, J = 8.1 Hz, 1H), 4.43 (d, J = 18.4 Hz, 1H), 4.36 (d, J = 18.4 Hz, 1H), 3.56-3.34 (m, 1H), 2.47 (s, 3H), 1.32 (d, J = 6.7 Hz, 6H). |
| 144 | 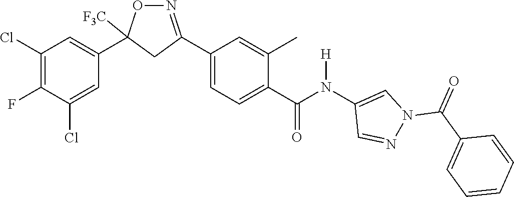 [SMILES: Cc1cc(C2=NOC(c3cc(Cl)c(F)c(Cl)c3)(C(F)(F)F)C2)ccc1C(=O)Nc1cnn(C(=O)c2ccccc2)c1] | ¹H NMR (600 MHz, DMSO-d₆) δ 10.31 (s, 1H), 8.61 (s, 1H), 8.04 (d, J = 7.7 Hz, 2H), 7.94 (s, 1H), 7.83-7.79 (m, 3H), 7.74-7.65 (m, 4H), 7.60 (d, J = 8.1 Hz, 1H), 4.41 (d, J = 18.4 Hz, 1H), 4.33 (d, J = 18.4 Hz, 1H), 2.42 (s, 3H). |
| 145 | 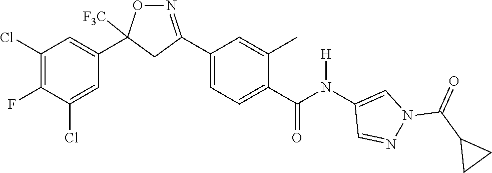 [SMILES: Cc1cc(C2=NOC(c3cc(Cl)c(F)c(Cl)c3)(C(F)(F)F)C2)ccc1C(=O)Nc1cnn(C(=O)C2CC2)c1] | ¹H NMR (600 MHz, DMSO-d₆) δ 10.33 (s, 1H), 8.73 (s, 1H), 7.86 (s, 1H), 7.81 (d, J = 6.3 Hz, 2H), 7.64 (s, 1H), 7.61 (d, J = 8.1 Hz, 1H), 7.58 (d, J = 8.2 Hz, 1H), 4.41 (d, J = 18.4 Hz, 1H), 4.35 (d, J = 18.4 Hz, 1H), 3.13-3.04 (m, 1H), 2.43 (s, 3H), 0.99-0.93 (m, 2H), 0.91- 0.85 (m, 2H). |
| 146 | 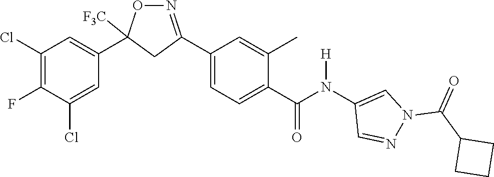 [SMILES: Cc1cc(C2=NOC(c3cc(Cl)c(F)c(Cl)c3)(C(F)(F)F)C2)ccc1C(=O)Nc1cnn(C(=O)C2CCC2)c1] | ¹H NMR (400 MHz, DMSO-d₆) δ 10.41 (s, 1H), 9.82 (s, 1H), 7.86 (s, 1H), 7.83 (s, 1H), 7.63 (s, 2H), 7.61 (d, J = 1.9 Hz, 1H), 7.56 (s, 1H), 4.42 (d, J = 18.4 Hz, 1H), 4.35 (d, J = 18.4 Hz, 1H), 2.43 (s, 3H), 2.03- 1.95 (m, 1H), 1.92-1.86 (m, 2H), 1.82-1.77 (m, 2H), 1.72- 1.65 (m, 2H). |
| 147 | 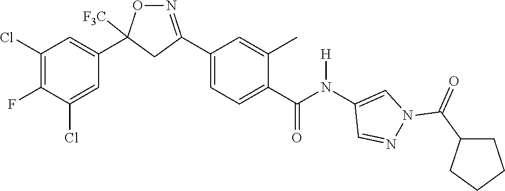 [SMILES: Cc1cc(C2=NOC(c3cc(Cl)c(F)c(Cl)c3)(C(F)(F)F)C2)ccc1C(=O)Nc1cnn(C(=O)C2CCCC2)c1] | ¹H NMR (400 MHz, DMSO-d₆) δ 10.45 (s, 1H), 9.84 (s, 1H), 7.83 (s, 1H), 7.82 (s, 1H), 7.66 (s, 2H), 7.63 (d, J = 1.7 Hz, 1H), 7.58 (s, 1H), 4.41 (d, J = 18.4 Hz, 1H), 4.34 (d, J = 18.4 Hz, 1H), 2.71-2.66 (m, 1H), 2.42 (s, 3H), 1.86-1.75 (m, 2H), 1.72-1.60 (m, 4H), 1.59- 1.48 (m, 2H). |
| 148 | 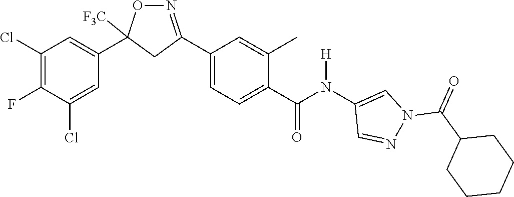 [SMILES: Cc1cc(C2=NOC(c3cc(Cl)c(F)c(Cl)c3)(C(F)(F)F)C2)ccc1C(=O)Nc1cnn(C(=O)C2CCCCC2)c1] | ¹H NMR (400 MHz, DMSO-d₆) δ 10.43 (s, 1H), 9.83 (s, 1H), 7.84 (s, 1H), 7.81 (s, 1H), 7.68 (s, 2H), 7.65 (d, J = 1.7 Hz, 1H), 7.59 (s, 1H), 4.39 (d, J = 18.4 Hz, 1H), 4.34 (d, J = 18.4 Hz, 1H), 2.75-2.69 (m, 1H), 2.46 (s, 3H), 1.52-1.45 (m, 2H), 1.42-1.36 (m, 2H), 1.32- 1.26 (m, 4H), 1.12-1.05 (m, 2H). |
| 149 | 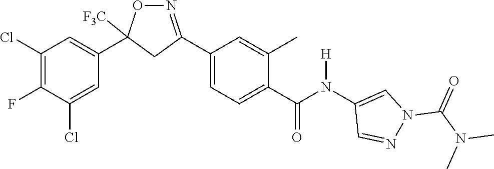 [SMILES: Cc1cc(C2=NOC(c3cc(Cl)c(F)c(Cl)c3)(C(F)(F)F)C2)ccc1C(=O)Nc1cnn(C(=O)N(C)C)c1] | ¹H NMR (600 MHz, DMSO-d₆) δ 10.69 (s, 1H), 8.41 (s, 1H), 7.94- 7.77 (m, 3H), 7.73-7.53 (m, 3H), 4.41 (d, J = 18.4 Hz, 1H), 4.35 (d, J = 18.4 Hz, 1H), 3.14 (s, 6H), 2.44 (s, 3H). |
| 150 | 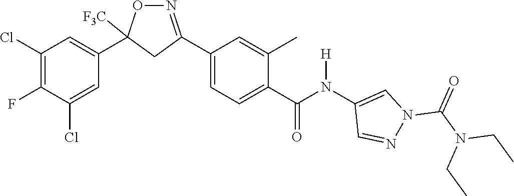 [SMILES: CCN(CC)C(=O)n1cc(NC(=O)c2ccc(C3=NOC(c4cc(Cl)c(F)c(Cl)c4)(C(F)(F)F)C3)cc2C)cn1] | ¹H NMR (600 MHz, DMSO-d₆) δ 10.72 (s, 1H), 8.43 (d, J = 4.1 Hz, 1H), 7.87-7.79 (m, 3H), 7.67 (d, J = 7.6 Hz, 2H), 7.61 (d, J = 7.4 Hz, 1H), 4.41 (d, J = 18.4 Hz, 1H), 4.35 (d, J = 18.4 Hz, 1H), 3.52 (q, J = 6.3 Hz, 4H), 2.43 (s, 3H), 1.20 (t, J = 6.3 Hz, 6H). |
| 151 | 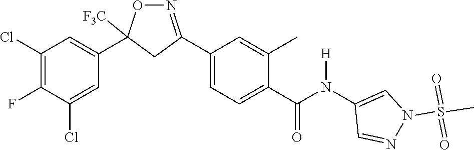 [SMILES: Cc1cc(C2=NOC(c3cc(Cl)c(F)c(Cl)c3)(C(F)(F)F)C2)ccc1C(=O)Nc1cnn(S(C)(=O)=O)c1] | ¹H NMR (600 MHz, DMSO-d₆) δ 10.86 (s, 1H), 8.47 (s, 1H), 8.02 (s, 1H), 7.82 (d, J = 5.9 Hz, 2H), 7.67 (d, J = 8.6 Hz, 2H), 7.62 (d, J = 7.9 Hz, 1H), 4.40 (d, J = 18.4 Hz, 1H), 4.34 (d, J = 18.4 Hz, 1H), 3.55 (s, 3H), 2.44 (s, 3H). |
| 152 | 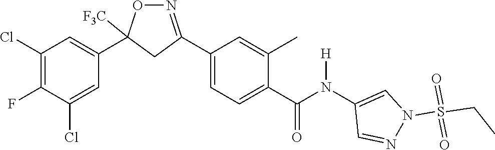 [SMILES: CCS(=O)(=O)n1cc(NC(=O)c2ccc(C3=NOC(c4cc(Cl)c(F)c(Cl)c4)(C(F)(F)F)C3)cc2C)cn1] | ¹H NMR (600 MHz, DMSO-d₆) δ 10.85 (s, 1H), 8.47 (s, 1H), 8.04 (s, 1H), 7.82 (d, J = 6.0 Hz, 2H), 7.67 (d, J = 8.6 Hz, 2H), 7.63 (d, J = 7.9 Hz, 1H), 4.41 (d, J = 18.4 Hz, 1H), 4.34 (d, J = 18.4 Hz, 1H), 3.72 (q, J = 7.3 Hz, 2H), 2.44 (s, 3H), 1.13 (t, J = 7.3 Hz, 3H). |
| 153 | 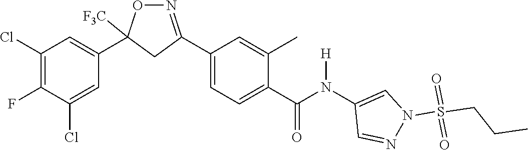 [SMILES: CCCS(=O)(=O)n1cc(NC(=O)c2ccc(C3=NOC(c4cc(Cl)c(F)c(Cl)c4)(C(F)(F)F)C3)cc2C)cn1] | ¹H NMR (600 MHz, DMSO-d₆) δ 10.86 (s, 1H), 8.47 (s, 1H), 8.03 (s, 1H), 7.82 (d, J = 4.4 Hz, 2H), 7.68 (d, J = 8.4 Hz, 2H), 7.63 (d, J = 6.6 Hz, 1H), 4.40 (d, J = 18.4 Hz, 1H), 4.35 (d, J = 18.4 Hz, 1H), 3.68 (t, J = 8.8 Hz, 2H), 2.44 (s, 3H), 1.61 - 1.53 (m, 2H), 0.92 (t, J = 7.8 Hz, 3H). |
| 154 | 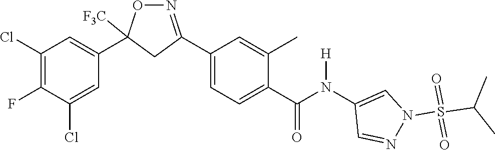 [SMILES: Cc1cc(C2=NOC(c3cc(Cl)c(F)c(Cl)c3)(C(F)(F)F)C2)ccc1C(=O)Nc1cnn(S(=O)(=O)C(C)C)c1] | ¹H NMR (600 MHz, DMSO-d₆) δ 10.87 (s, 1H), 8.48 (s, 1H), 8.06 (s, 1H), 7.83 (d, J = 5.9 Hz, 2H), 7.68 (d, J = 8.4 Hz, 2H), 7.64 (d, J = 8.0 Hz, 1H), 4.41 (d, J = 18.4 Hz, 1H), 4.35 (d, J = 18.4 Hz, 1H), 3.95- 3.87 (m, 1H), 2.45 (s, 3H), 1.25 (d, J = 6.9 Hz, 6H). |
| 155 | 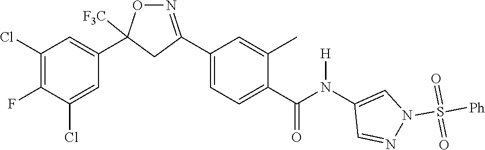 [SMILES: Cc1cc(C2=NOC(c3cc(Cl)c(F)c(Cl)c3)(C(F)(F)F)C2)ccc1C(=O)Nc1cnn(S(=O)(=O)c2ccccc2)c1] | ¹H NMR (600 MHz, DMSO-d₆) δ 10.84 (s, 1H), 8.60 (s, 1H), 8.01 (d, J = 7.8 Hz, 2H), 7.97 (s, 1H), 7.84-7.78 (m, 3H), 7.72-7.63 (m, 4H), 7.60 (d, J = 8.2 Hz, 1H), 4.40 (d, J = 18.4 Hz, 1H), 4.33 (d, J = 18.4 Hz, 1H), 2.42 (s, 3H). |
| 156 | 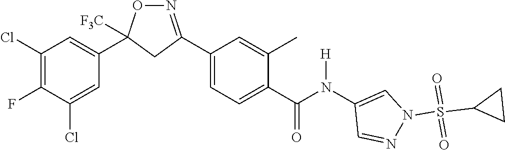 [SMILES: Cc1cc(C2=NOC(c3cc(Cl)c(F)c(Cl)c3)(C(F)(F)F)C2)ccc1C(=O)Nc1cnn(S(=O)(=O)C2CC2)c1] | ¹H NMR (600 MHz, DMSO-d₆) δ 10.87 (s, 1H), 8.47 (s, 1H), 8.04 (s, 1H), 7.83 (d, J = 6.0 Hz, 2H), 7.68 (d, J = 8.6 Hz, 2H), 7.64 (d, J = 8.0 Hz, 1H), 4.41 (d, J = 18.4 Hz, 1H), 4.35 (d, J = 18.4 Hz, 1H), 3.20- 3.13 (m, 1H), 2.45 (s, 3H), 1.30-1.26 (m, 2H), 1.22-1.20 (m, 2H). |
| 157 | 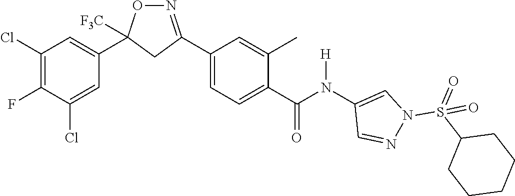 [SMILES: Cc1cc(C2=NOC(c3cc(Cl)c(F)c(Cl)c3)(C(F)(F)F)C2)ccc1C(=O)Nc1cnn(S(=O)(=O)C2CCCCC2)c1] | ¹H NMR (600 MHz, DMSO-d₆) δ 10.87 (s, 1H), 8.45 (s, 1H), 8.04 (s, 1H), 7.86-7.76 (m, 2H), 7.74-7.58 (m, 3H), 4.41 (d, J = 18.4 Hz, 1H), 4.35 (d, J = 18.4 Hz, 1H), 3.76-3.64 (m, 1H), 2.44 (s, 3H), 1.87- 1.80 (m, 2H), 1.80-1.72 (m, 2H), 1.43-1.33 (m, 2H), 1.32-1.22 (m, 2H), 1.21-1.13 (m, 2H). |
| 158 | 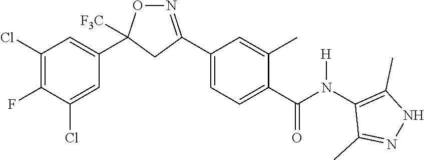 [SMILES: Cc1cc(C2=NOC(c3cc(Cl)c(F)c(Cl)c3)(C(F)(F)F)C2)ccc1C(=O)Nc1c(C)n[nH]c1C] | ¹H NMR (600 MHz, DMSO-d₆) δ 12.16 (s, 1H), 9.43 (s, 1H), 7.83 (d, J = 6.0 Hz, 2H), 7.64 (d, J = 9.2 Hz, 2H), 7.57 (d, J = 7.8 Hz, 1H), 4.40 (d, J = 18.4 Hz, 1H), 4.34 (d, J = 18.4 Hz, 1H), 2.45 (s, 3H), 2.09 (s, 6H). |
| 159 | 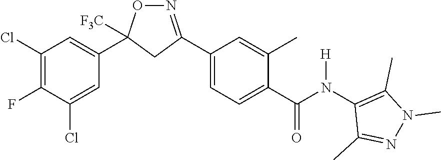 [SMILES: Cc1cc(C2=NOC(c3cc(Cl)c(F)c(Cl)c3)(C(F)(F)F)C2)ccc1C(=O)Nc1c(C)nn(C)c1C] | ¹H NMR (600 MHz, DMSO-d₆) δ 9.48 (s, 1H), 7.83 (d, J = 6.0 Hz, 2H), 7.64 (d, J = 9.3 Hz, 2H), 7.57 (d, J = 7.9 Hz, 1H), 4.40 (d, J = 18.4 Hz, 1H), 4.34 (d, J = 18.4 Hz, 1H), 3.66 (s, 3H), 2.45 (s, 3H), 2.13 (s, 3H), 2.04 (s, 3H). |
| 160 | 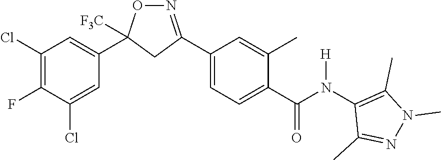 [SMILES: Cc1cc(C2=NOC(c3cc(Cl)c(F)c(Cl)c3)(C(F)(F)F)C2)ccc1C(=O)Nc1c(C)nn(C)c1C] | ¹H NMR (600 MHz, DMSO-d₆) δ 9.46 (s, 1H), 7.83 (d, J = 6.1 Hz, 2H), 7.65 (d, J = 9.3 Hz, 2H), 7.58 (d, J = 7.8 Hz, 1H), 4.40 (d, J = 18.4 Hz, 1H), 4.34 (d, J = 18.4 Hz, 1H), 3.98 (q, J = 7.2 Hz, 2H), 2.45 (s, 3H), 2.14 (s, 3H), 2.05 (s, 3H), 1.28 (t, J = 7.2 Hz, 3H). |
| 161 | 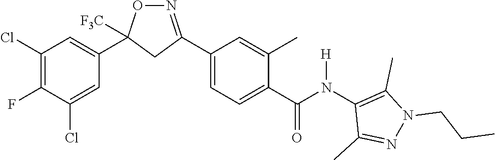 [SMILES: CCCn1nc(C)c(NC(=O)c2ccc(C3=NOC(c4cc(Cl)c(F)c(Cl)c4)(C(F)(F)F)C3)cc2C)c1C] | ¹H NMR (600 MHz, DMSO-d₆) δ 9.45 (s, 1H), 7.82 (d, J = 6.0 Hz, 2H), 7.64 (d, J = 9.6 Hz, 2H), 7.57 (d, J = 7.8 Hz, 1H), 4.39 (d, J = 18.4 Hz, 1H), 4.34 (d, J = 18.4 Hz, 1H), 3.90 (t, J = 6.8 Hz, 2H), 2.45 (s, 3H), 2.14 (s, 3H), 2.05 (s, 3H), 1.74-1.68 (m, 2H), 0.85 (t, J = 7.4 Hz, 3H). |
| 162 | 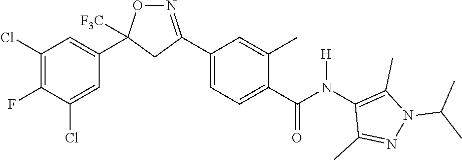 [SMILES: Cc1cc(C2=NOC(c3cc(Cl)c(F)c(Cl)c3)(C(F)(F)F)C2)ccc1C(=O)Nc1c(C)nn(C(C)C)c1C] | ¹H NMR (600 MHz, DMSO-d₆) δ 9.44 (s, 1H), 7.83 (d, J = 6.0 Hz, 2H), 7.65 (d, J = 9.3 Hz, 2H), 7.57 (d, J = 7.8 Hz, 1H), 4.46-4.42 (m, 1H), 4.40 (d, J = 18.4 Hz, 1H), 4.34 (d, J = 18.4 Hz, 1H), 2.45 (s, 3H), 2.15 (s, 3H), 2.06 (s, 3H), 1.35 (d, J = 6.6 Hz, 6H). |
| 163 | 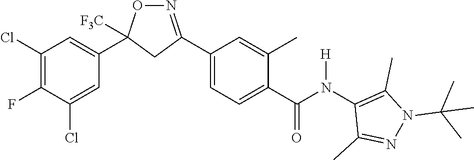 [SMILES: Cc1cc(C2=NOC(c3cc(Cl)c(F)c(Cl)c3)(C(F)(F)F)C2)ccc1C(=O)Nc1c(C)nn(C(C)(C)C)c1C] | ¹H NMR (600 MHz, Deuterium Oxide) δ 9.42 (s, 1H), 7.83 (d, J = 6.0 Hz, 2H), 7.65 (d, J = 9.1 Hz, 2H), 7.57 (d, J = 7.8 Hz, 1H), 4.39 (d, J = 18.4 Hz, 1H), 4.34 (d, J = 18.4 Hz, 1H), 2.45 (s, 3H), 2.28 (s, 3H), 2.04 (s, 3H), 1.55 (s, 9H). |
| 164 | 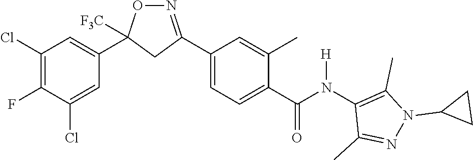 [SMILES: Cc1cc(C2=NOC(c3cc(Cl)c(F)c(Cl)c3)(C(F)(F)F)C2)ccc1C(=O)Nc1c(C)nn(C2CC2)c1C] | ¹H NMR (400 MHz, DMSO-d₆) δ 9.48 (s, 1H), 7.86-7.80 (m, 2H), 7.64 (d, J = 7.5 Hz, 2H), 7.57 (d, J = 7.9 Hz, 1H), 4.40 (d, J = 18.4 Hz, 1H), 4.33 (d, J = 18.4 Hz, 1H), 3.46-3.37 (m, 1H), 2.44 (s, 3H), 2.21 (s, 3H), 2.03 (s, 3H), 1.02 - 0.97 (m, 2H), 0.97-0.93 (m, 2H). |
| 165 | 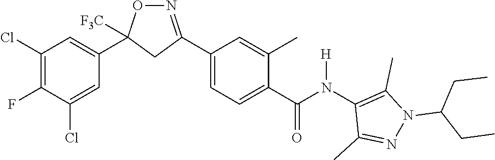 [SMILES: CCC(CC)n1nc(C)c(NC(=O)c2ccc(C3=NOC(c4cc(Cl)c(F)c(Cl)c4)(C(F)(F)F)C3)cc2C)c1C] | ¹H NMR (600 MHz, DMSO-d₆) δ 9.45 (s, 1H), 7.83 (d, J = 6.1 Hz, 2H), 7.64 (d, J = 10.5 Hz, 2H), 7.58 (d, J = 7.9 Hz, 1H), 4.40 (d, J = 18.4 Hz, 1H), 4.34 (d, J = 18.4 Hz, 1H), 3.92-3.82 (m, 1H), 2.46 (s, 3H), 2.13 (s, 3H), 2.08 (s, 3H), 1.85-1.77 (m, 2H), 1.74-1.67 (m, 2H), 0.68 (t, J = 7.3 Hz, 6H). |
| 166 | 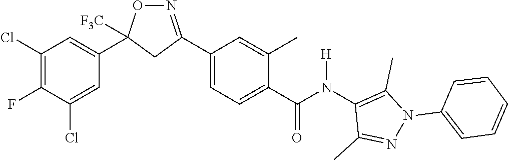 [SMILES: Cc1cc(C2=NOC(c3cc(Cl)c(F)c(Cl)c3)(C(F)(F)F)C2)ccc1C(=O)Nc1c(C)nn(-c2ccccc2)c1C] | ¹H NMR (600 MHz, DMSO-d₆) δ 9.70 (s, 1H), 7.83 (d, J = 6.1 Hz, 2H), 7.68 (d, J = 8.6 Hz, 2H), 7.63 (d, J = 7.8 Hz, 1H), 7.56-7.50 (m, 4H), 7.43-7.38 (m, 1H), 4.41 (d, J = 18.4 Hz, 1H), 4.35 (d, J = 18.4 Hz, 1H), 2.49 (s, 3H), 2.26 (s, 3H), 2.18 (s, 3H). |
| 167 | 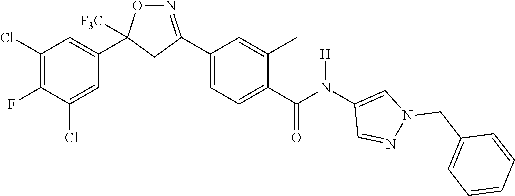 [SMILES: Cc1cc(C2=NOC(c3cc(Cl)c(F)c(Cl)c3)(C(F)(F)F)C2)ccc1C(=O)Nc1cnn(Cc2ccccc2)c1] | ¹H NMR (400 MHz, Chloroform-d) δ 7.59 (d, J = 6.0 Hz, 2H), 7.56 (s, 3H), 7.36-7.27 (m, 3H), 7.17-7.11 (m, 2H), 6.90 (s, 1H), 5.25 (s, 2H), 4.10 (d, J = 17.4 Hz, 1H), 3.71 (d, J = 17.4 Hz, 1H), 2.55 (s, 3H), 2.24 (s, 3H), 2.15 (s, 3H). |
| 168 | 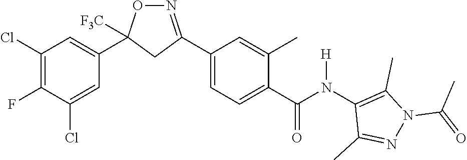 [SMILES: CC(=O)n1nc(C)c(NC(=O)c2ccc(C3=NOC(c4cc(Cl)c(F)c(Cl)c4)(C(F)(F)F)C3)cc2C)c1C] | ¹H NMR (600 MHz, DMSO-d₆) δ 9.85 (s, 1H), 7.83 (d, J = 6.0 Hz, 2H), 7.65 (s, 3H), 4.41 (d, J = 18.4 Hz, 1H), 4.35 (d, J = 18.4 Hz, 1H), 2.60 (s, 3H), 2.46 (s, 3H), 2.41 (s, 3H), 2.18 (s, 3H). |
| 169 | 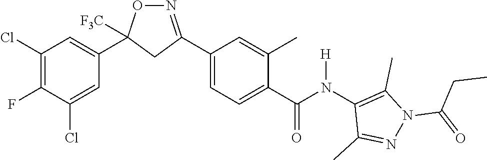 [SMILES: CCC(=O)n1nc(C)c(NC(=O)c2ccc(C3=NOC(c4cc(Cl)c(F)c(Cl)c4)(C(F)(F)F)C3)cc2C)c1C] | ¹H NMR (400 MHz, DMSO-d₆) δ 9.84 (s, 1H), 7.85 (d, J = 6.2 Hz, 2H), 7.73-7.65 (m, 3H), 4.41 (d, J = 18.4 Hz, 1H), 4.35 (d, J = 18.4 Hz, 1H), 2.96 (q, J = 7.3 Hz, 2H), 2.46 (s, 3H), 2.42 (s, 3H), 2.16 (s, 3H), 1.32 (t, J = 7.4 Hz, 3H). |
| 170 | 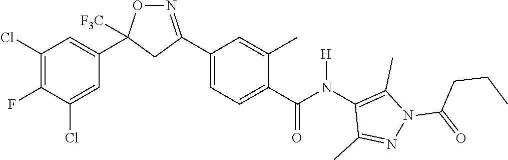 [SMILES: CCCC(=O)n1nc(C)c(NC(=O)c2ccc(C3=NOC(c4cc(Cl)c(F)c(Cl)c4)(C(F)(F)F)C3)cc2C)c1C] | ¹H NMR (400 MHz, DMSO-d₆) δ 9.85 (s, 1H), 7.84 (d, J = 6.2 Hz, 2H), 7.71-7.63 (m, 3H), 4.42 (d, J = 18.4 Hz, 1H), 4.35 (d, J = 18.4 Hz, 1H), 3.07 (t, J = 7.3 Hz, 2H), 2.47 (s, 3H), 2.43 (s, 3H), 2.19 (s, 3H), 1.76-1.61 (m, 2H), 0.98 (t, J = 7.4 Hz, 3H). |
| 171 | 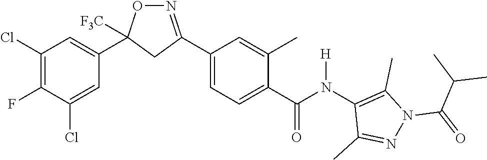 [SMILES: Cc1cc(C2=NOC(c3cc(Cl)c(F)c(Cl)c3)(C(F)(F)F)C2)ccc1C(=O)Nc1c(C)nn(C(=O)C(C)C)c1C] | ¹H NMR (400 MHz, DMSO-d₆) δ 9.83 (s, 1H), 7.86 (d, J = 6.2 Hz, 2H), 7.69-7.61 (m, 3H), 4.41 (d, J = 18.4 Hz, 1H), 4.33 (d, J = 18.4 Hz, 1H), 3.28-3.16 (m, 1H), 2.49 (s, 3H), 2.44 (s, 3H), 2.21 (s, 3H), 1.27 (d, J = 6.6 Hz, 6H). |
| 172 | 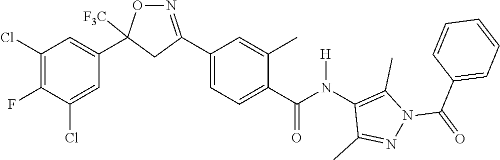 [SMILES: Cc1cc(C2=NOC(c3cc(Cl)c(F)c(Cl)c3)(C(F)(F)F)C2)ccc1C(=O)Nc1c(C)nn(C(=O)c2ccccc2)c1C] | ¹H NMR (600 MHz, DMSO-d₆) δ 9.83 (s, 1H), 7.96 (d, J = 7.6 Hz, 2H), 7.80 (d, J = 5.4 Hz, 2H), 7.79 (s, 1H), 7.71 (s, 2H), 7.66 (d, J = 10.8 Hz, 2H), 7.60 (d, J = 7.8 Hz, 1H), 4.41 (d, J = 18.4 Hz, 1H), 4.35 (d, J = 18.4 Hz, 1H), 2.47 (s, 3H), 2.43 (s, 3H), 2.19 (s, 3H). |
| 173 | 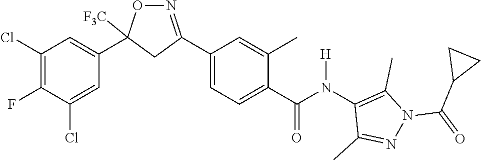 [SMILES: Cc1cc(C2=NOC(c3cc(Cl)c(F)c(Cl)c3)(C(F)(F)F)C2)ccc1C(=O)Nc1c(C)nn(C(=O)C2CC2)c1C] | ¹H NMR (600 MHz, DMSO-d₆) δ 9.86 (s, 1H), 7.83 (d, J = 6.3 Hz, 2H), 7.68 (s, 1H), 7.67-7.63 (m, 2H), 4.41 (d, J = 18.4 Hz, 1H), 4.35 (d, J = 18.4 Hz, 1H), 3.15-3.10 (m, 1H), 2.46 (s, 3H), 2.40 (s, 3H), 2.21 (s, 3H), 1.13-1.08 (m, 2H), 1.08-1.04 (m, 2H). |
| 174 | 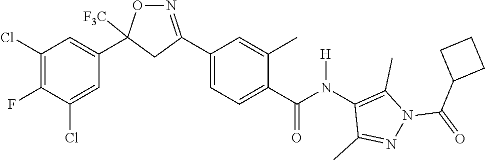 [SMILES: Cc1cc(C2=NOC(c3cc(Cl)c(F)c(Cl)c3)(C(F)(F)F)C2)ccc1C(=O)Nc1c(C)nn(C(=O)C2CCC2)c1C] | ¹H NMR (600 MHz, DMSO-d₆) δ 9.82 (s, 1H), 7.84 - 7.80 (m, 2H), 7.66 (d, J = 8.9 Hz, 2H), 7.63 (d, J = 7.8 Hz, 1H), 4.40 (d, J = 18.4 Hz, 1H), 4.34 (d, J = 18.4 Hz, 1H), 4.25-4.20 (m, 1H), 2.45 (s, 3H), 2.42 (s, 3H), 2.32-2.27 (m, 2H), 2.27-2.19 (m, 2H), 2.15 (s, 3H), 2.06- 2.00 (m, 1H), 1.87-1.79 (m, 1H). |
| 175 | 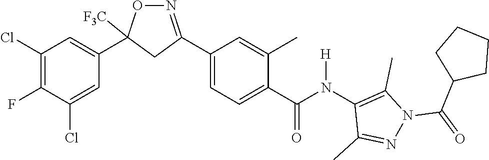 [SMILES: Cc1cc(C2=NOC(c3cc(Cl)c(F)c(Cl)c3)(C(F)(F)F)C2)ccc1C(=O)Nc1c(C)nn(C(=O)C2CCCC2)c1C] | ¹H NMR (600 MHz, DMSO-d₆) δ 9.83 (s, 1H), 7.82 (d, J = 5.7 Hz, 2H), 7.67 (s, 1H), 7.66 (s, 1H), 7.64 (s, 1H), 4.40 (d, J = 18.4 Hz, 1H), 4.35 (d, J = 18.4 Hz, 1H), 4.00-3.94 (m, 1H), 2.46 (s, 3H), 2.41 (s, 3H), 2.18 (s, 3H), 1.81-1.73 (m, 2H), 1.73-1.67 (m, 2H), 1.66- 1.60 (m, 2H), 0.89-0.80 (m, 2H). |
| 176 | 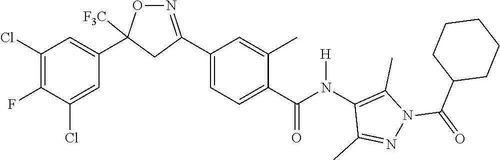 [SMILES: Cc1cc(C2=NOC(c3cc(Cl)c(F)c(Cl)c3)(C(F)(F)F)C2)ccc1C(=O)Nc1c(C)nn(C(=O)C2CCCCC2)c1C] | ¹H NMR (600 MHz, DMSO-d₆) δ 9.84 (s, 1H), 7.83 (d, J = 6.0 Hz, 2H), 7.67 (d, J = 9.2 Hz, 2H), 7.63 (d, J = 7.8 Hz, 1H), 4.40 (d, J = 18.4 Hz, 1H), 4.35 (d, J = 18.4 Hz, 1H), 3.68-3.56 (m, 1H), 2.46 (s, 3H), 2.40 (s, 3H), 2.18 (s, 3H), 1.94-1.88 (m, 2H), 1.80-1.74 (m, 2H), 1.72-1.65 (m, 2H), 1.45-1.40 (m, 2H), 1.39-1.33 (m, 2H). |
| 177 | 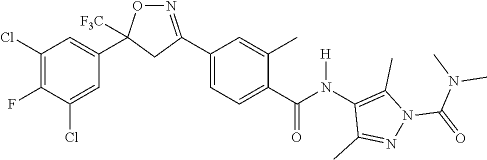 [SMILES: Cc1cc(C2=NOC(c3cc(Cl)c(F)c(Cl)c3)(C(F)(F)F)C2)ccc1C(=O)Nc1c(C)nn(C(=O)N(C)C)c1C] | ¹H NMR (600 MHz, DMSO-d₆) δ 9.68 (s, 1H), 7.83 (d, J = 6.1 Hz, 2H), 7.67 (d, J = 9.4 Hz, 2H), 7.62 (d, J = 7.8 Hz, 1H), 4.40 (d, J = 18.4 Hz, 1H), 4.34 (d, J = 18.4 Hz, 1H), 3.03 (s, 6H), 2.47 (s, 3H), 2.25 (s, 3H), 2.13 (s, 3H). |
| 178 | 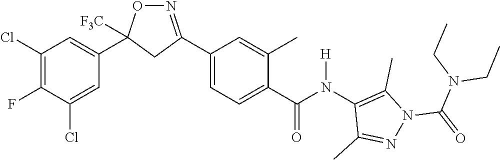 [SMILES: CCN(CC)C(=O)n1nc(C)c(NC(=O)c2ccc(C3=NOC(c4cc(Cl)c(F)c(Cl)c4)(C(F)(F)F)C3)cc2C)c1C] | ¹H NMR (400 MHz, DMSO-d₆) δ 9.68 (s, 1H), 7.83 (s, 1H), 7.82 (s, 1H), 7.67 (s, 1H), 7.65 (d, J = 1.6 Hz, 1H), 7.63-7.60 (m, 1H), 4.38 (d, J = 18.4 Hz, 1H), 4.34 (d, J = 18.4 Hz, 1H), 3.38 (q, J = 7.0 Hz, 4H), 2.46 (s, 3H), 2.23 (s, 3H), 2.13 (s, 3H), 1.16 (t, J = 7.0 Hz, 6H). |
| 179 | 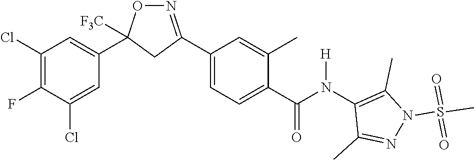 [SMILES: Cc1cc(C2=NOC(c3cc(Cl)c(F)c(Cl)c3)(C(F)(F)F)C2)ccc1C(=O)Nc1c(C)nn(S(C)(=O)=O)c1C] | ¹H NMR (600 MHz, DMSO-d₆) δ 9.85 (s, 1H), 7.83 (d, J = 6.1 Hz, 2H), 7.68 (s, 1H), 7.65 (t, J = 9.0 Hz, 2H), 4.41 (d, J = 18.4 Hz, 1H), 4.35 (d, J = 18.4 Hz, 1H), 3.51 (s, 3H), 2.47 (s, 3H), 2.38 (s, 3H), 2.18 (s, 3H). |
| 180 | 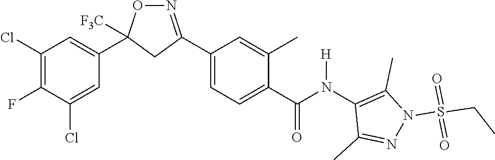 [SMILES: CCS(=O)(=O)n1nc(C)c(NC(=O)c2ccc(C3=NOC(c4cc(Cl)c(F)c(Cl)c4)(C(F)(F)F)C3)cc2C)c1C] | ¹H NMR (600 MHz, DMSO-d₆) δ 9.86 (s, 1H), 7.83 (d, J = 6.0 Hz, 2H), 7.68 (s, 1H), 7.66 (d, J = 5.2 Hz, 2H), 4.41 (d, J = 18.4 Hz, 1H), 4.35 (d, J = 18.4 Hz, 1H), 3.65 (q, J = 7.3 Hz, 2H), 2.46 (s, 3H), 2.37 (s, 3H), 2.18 (s, 3H), 1.13 (t, J = 7.3 Hz, 3H). |
| 181 | 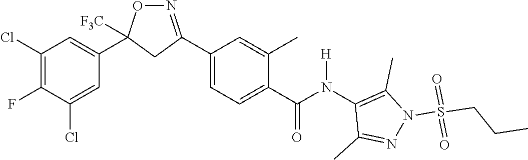 [SMILES: CCCS(=O)(=O)n1nc(C)c(NC(=O)c2ccc(C3=NOC(c4cc(Cl)c(F)c(Cl)c4)(C(F)(F)F)C3)cc2C)c1C] | ¹H NMR (600 MHz, DMSO-d₆) δ 9.84 (s, 1H), 7.85-7.80 (m, 2H), 7.66 (d, J = 13.4 Hz, 3H), 4.40 (d, J = 18.4 Hz, 1H), 4.34 (d, J = 18.4 Hz, 1H), 3.62 (t, J = 8.6 Hz, 2H), 2.46 (s, 3H), 2.37 (s, 3H), 2.17 (s, 3H), 1.62-1.56 (m, 2H), 0.94 (t, J = 7.7 Hz, 3H). |
| 182 | 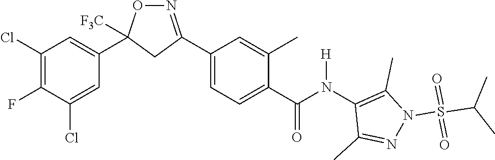 [SMILES: Cc1cc(C2=NOC(c3cc(Cl)c(F)c(Cl)c3)(C(F)(F)F)C2)ccc1C(=O)Nc1c(C)nn(S(=O)(=O)C(C)C)c1C] | ¹H NMR (600 MHz, DMSO-d₆) δ 9.83 (s, 1H), 7.80 (d, J = 5.9 Hz, 2H), 7.64 (d, J = 11.4 Hz, 3H), 4.38 (d, J = 18.4 Hz, 1H), 4.32 (d, J = 18.4 Hz, 1H), 3.85 - 3.75 (m, 1H), 2.44 (s, 3H), 2.34 (s, 3H), 2.15 (s, 3H), 1.20 (t, J = 5.2 Hz, 6H). |
| 183 | 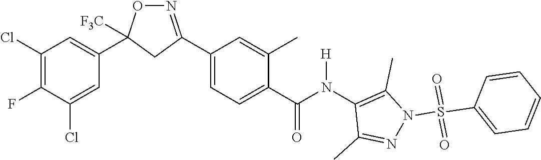 [SMILES: Cc1cc(C2=NOC(c3cc(Cl)c(F)c(Cl)c3)(C(F)(F)F)C2)ccc1C(=O)Nc1c(C)nn(S(=O)(=O)c2ccccc2)c1C] | ¹H NMR (600 MHz, DMSO-d₆) δ 9.79 (s, 1H), 7.98 (d, J = 7.7 Hz, 2H), 7.81 (d, J = 5.2 Hz, 2H), 7.79 (s, 1H), 7.69 (s, 2H), 7.64 (d, J = 11.2 Hz, 2H), 7.61 (d, J = 7.8 Hz, 1H), 4.39 (d, J = 18.4 Hz, 1H), 4.33 (d, J = 18.4 Hz, 1H), 2.42 (s, 6H), 2.10 (s, 3H). |
| 184 | 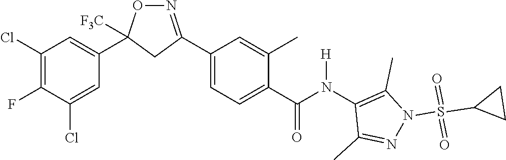 [SMILES: Cc1cc(C2=NOC(c3cc(Cl)c(F)c(Cl)c3)(C(F)(F)F)C2)ccc1C(=O)Nc1c(C)nn(S(=O)(=O)C2CC2)c1C] | ¹H NMR (600 MHz, DMSO-d₆) δ 9.83 (s, 1H), 7.82 (d, J = 6.1 Hz, 2H), 7.68 (s, 1H), 7.65 (t, J = 7.5 Hz, 2H), 4.40 (d, J = 18.4 Hz, 1H), 4.34 (d, J = 18.4 Hz, 1H), 3.19-3.14 (m, 1H), 2.46 (s, 3H), 2.37 (s, 3H), 2.27-2.20 (m, 1H), 2.18 (s, 3H), 2.12-2.07 (m, 1H), 1.19-1.15 (m, 2H). |
| 185 | 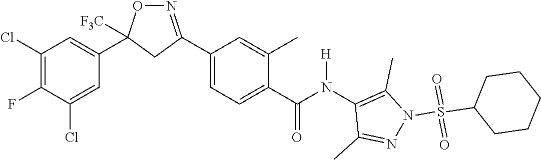 [SMILES: Cc1cc(C2=NOC(c3cc(Cl)c(F)c(Cl)c3)(C(F)(F)F)C2)ccc1C(=O)Nc1c(C)nn(S(=O)(=O)C2CCCCC2)c1C] | ¹H NMR (400 MHz, DMSO-d₆) δ 9.86 (s, 1H), 7.84 (d, J = 6.2 Hz, 2H), 7.71-7.66 (m, 3H), 4.43 (d, J = 18.4 Hz, 1H), 4.36 (d, J = 18.4 Hz, 1H), 3.71-3.61 (m, 1H), 2.48 (s, 3H), 2.38 (s, 3H), 2.20 (s, 3H), 1.84-1.77 (m, 4H), 1.66-1.58 (m, 1H), 1.51-1.40 (m, 2H), 1.33- 1.25 (m, 2H), 1.20-1.10 (m, 1H). |

### Preparation Example 1

This preparation serves to illustrate the synthesis of compound 1:

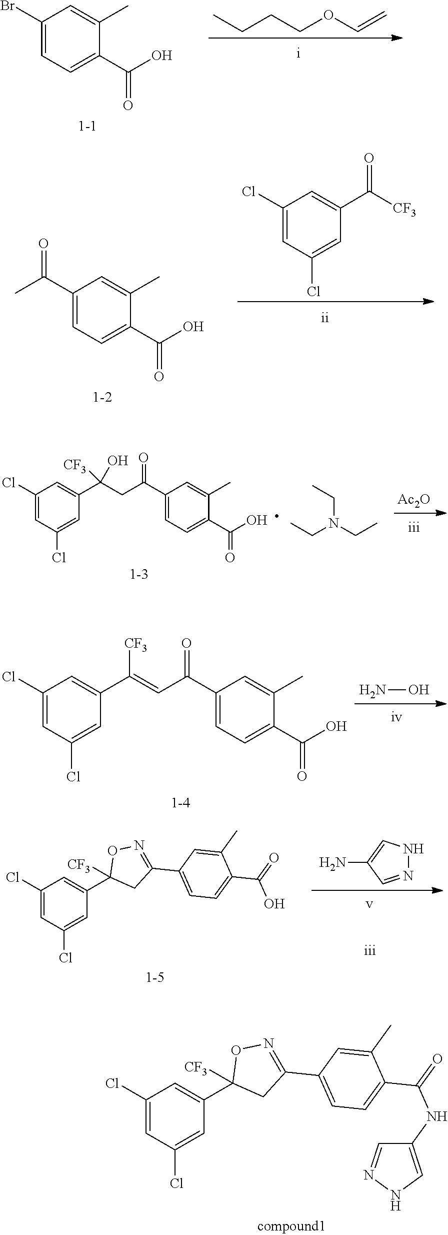

Preparation of intermediates 1-2 (step i): placing a compound (21.50 g) shown in the formula 1-1, palladium acetate (0.23 g) and potassium carbonate (2′7.64 g) in a Schlenk tube in sequence, pumping and filling nitrogen for three times, then adding n-butyl vinyl ether (30.00 g) and 100 mL of n-butanol, carrying out reflux reaction at 120° C. for 6 h, after the reaction is finished, evaporating the solvent to dryness under reduced pressure, adding a 1N HCl solution, acidifying to pH 2, carrying out suction filtration and drying to obtain an intermediate 1-2, wherein the yield is 92%.

Preparation of intermediates 1-3 (step ii): sequentially taking 1-2 (10.00 g) of the intermediate, 1-(3,5-dichlorophenyl)-2,2,2-trifluoroethanone (16.38 g), 11.37 g of triethylamine and 100 mL of toluene into a three-neck flask, reacting for 16 h at 50° C., cooling to room temperature after the reaction is finished, performing suction filtration, and drying the solid to obtain 1-3 of the intermediate, wherein the yield is 98%.

Preparation of intermediates 1-4 (step iii): sequentially taking the compound 1-3 (26.52 g), 4-dimethylaminopyridine (6.20 g) and toluene 100 mL into a three-necked bottle, adding acetic anhydride (10.36 g), reacting at 60° C. for 12 hours, adding a 1N HCl solution after the reaction is finished, acidifying until the pH value is 2, carrying out suction filtration and drying to obtain the intermediate 1-4, wherein the yield is 95%.

Preparation of intermediates 1-5 (step iv): sequentially taking the intermediate 1-4 (16.70 g), tetrabutylammonium bromide (4.01 g) and 300 mL of toluene into a three-neck bottle, adding 5.47 g of 50% hydroxylamine aqueous solution, reacting at room temperature for 7 hours, adding 1N HCl solution after the reaction is finished, acidifying until the pH value is 2, carrying out suction filtration and drying to obtain the intermediate 1-5, wherein the yield is 95%.

Preparation of compound 1 (step v): putting the intermediates 1-5 (0.50 g), CDI (0.39 g), DIPEA (0.56 g) and 10 mL of dichloromethane into a three-necked bottle in sequence, adding 4-aminopyrazole (0.16 g), reacting at normal temperature for 4 h, washing with saturated NaHCO₃ solution for 3 times after the reaction is finished, extracting an aqueous phase for 3 times with 20 mL of dichloromethane, merging organic phase after the extraction is finished, drying the organic phase with anhydrous Na₂SO₄, and purifying by column chromatography to obtain the compound 1 with the yield of 83%.

### Preparation Example 2

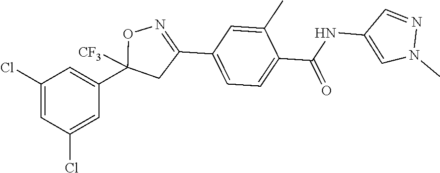 [SMILES: Cc1cc(C2=NOC(c3cc(Cl)cc(Cl)c3)(C(F)(F)F)C2)ccc1C(=O)Nc1cnn(C)c1]

This preparation serves to illustrate the synthesis of compound 2:

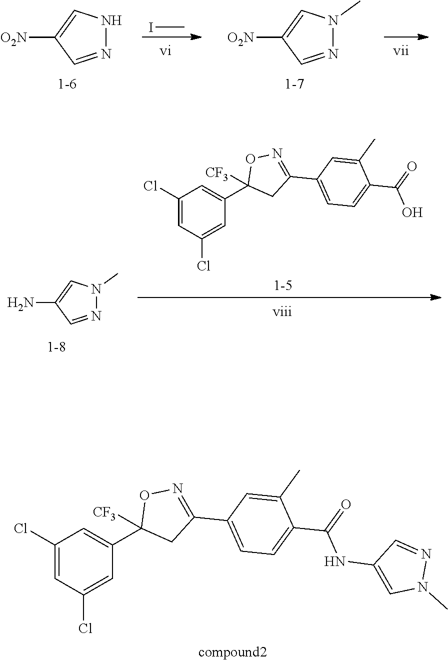

preparation of intermediates 1-7 (step vi): sequentially taking 20 mL of intermediates 1-6 (1.00 g), potassium carbonate (1.47 g), methyl iodide (1.50 g) and N, N-dimethylaminocarboxamide into a three-necked bottle, reacting at normal temperature for 3 h, adding 100 mL of ethyl acetate after the reaction is finished, washing the mixture with saturated saline solution for three times, drying an organic phase by using anhydrous Na₂SO₄, and purifying by column chromatography to obtain intermediates 1-7 with the yield of 93%.

Preparation of intermediates 1-8 (step vii): taking 1-7 intermediates (0.50 g), ammonium chloride (0.95 g), 5 mL of water and 15 mL of ethanol into a three-necked bottle in sequence, heating to boil, adding reduced iron powder (1.54 g) in batches, refluxing for 30 min, after the reaction is finished, adding 50 mL of ethyl acetate, washing with saturated saline solution for three times, drying an organic phase with anhydrous Na₂SO₄, and carrying out column chromatography purification to obtain 1-8 intermediates, wherein the yield is 78%.

Preparation of compound 2 (step viii): putting the intermediates 1-5 (0.50 g), CDI (0.39 g), DIPEA (0.56 g) and dichloromethane (10 mL) in a three-necked bottle in sequence, adding the intermediates 1-8 (0.18 g), reacting at normal temperature for 4 h, after the reaction is finished, washing with saturated NaHCO₃ solution for 3 times, extracting an aqueous phase for 3 times with 20 mL dichloromethane, combining organic phase after the extraction is finished, drying the organic phase with anhydrous Na₂SO₄, and purifying by column chromatography to obtain the compound 2, wherein the yield is 82%.

### Preparation of Compound 3-Compound 57, Compounds 76-94

Compound 3 to 57 and 76 to 94 were prepared according to a similar manner to that of preparation example 2.

### Preparation of Compound 58

Compound 58 is prepared similarly to preparation example 2, except that methyl iodide, potassium carbonate, and N, N-dimethylaminocarboxamide in step vi are replaced with acetyl chloride, triethylamine, and dichloromethane in equimolar amounts, and specifically, intermediates 1-7 are prepared by the steps comprising: sequentially taking the intermediate 1-6, triethylamine and dichloromethane into a three-necked bottle, dropwise adding acetyl chloride at 0° C., reacting at normal temperature for 3 h, after the reaction is finished, adding 100 mL of ethyl acetate, washing with saturated saline solution for three times, drying an organic phase with anhydrous Na₂SO₄, and purifying by column chromatography to obtain the intermediate 1-7 with the yield of 68%.

### Preparation of Compound 59-Compound 75

Compound 59-compound 75 were prepared according to a similar manner to that of compound 58.

### Preparation of Compound 95

Compound 95 is prepared analogously to preparation 1, except that the 4-aminopyrazole in step v is replaced with an equimolar amount of 3,5-dimethyl-1H-pyrazol-4-amine, specifically, in the preparation of compound 95: putting the intermediates 1-5 (0.50 g), CDI (0.39 g), DIPEA (0.56 g) and 10 mL of dichloromethane into a three-necked bottle in sequence, adding 3,5-dimethyl-1H-pyrazol-4-amine, reacting for 4H at normal temperature, washing 3 times with saturated NaHCO₃ solution after the reaction is finished, extracting an aqueous phase for 3 times with 20 mL of dichloromethane, combining organic phase after the extraction is finished, drying the organic layer with anhydrous Na₂SO₄, and purifying by column chromatography to obtain the compound 95, wherein the yield is 89%.

### Preparation of Compound 96

Compound 96 was prepared similarly to preparation 2, except that intermediates 1-6 in step vi were replaced with equimolar 3,5-dimethyl-4-nitropyrazole, specifically the preparation of intermediates 1-7: 3, 5-dimethyl-4-nitropyrazole, potassium carbonate (1.47 g), methyl iodide (1.50 g) and N,N-dimethylaminocarboxamide 20 mL are sequentially put into a three-necked bottle and reacted at normal temperature for 3 h, after the reaction is finished, 100 mL of ethyl acetate is added and washed with saturated saline three times, and then an organic phase is dried by anhydrous Na₂SO₄ and purified by column chromatography to obtain an intermediate 1-7 with the yield of 94%.

### Preparation of Compound 97

Compound 97 is prepared similarly to preparation 96, except that the iodomethane in step vi is replaced with an equimolar amount of iodoethane, and specifically, intermediates 1-7 are prepared by steps comprising:
sequentially taking 3,5-dimethyl-4-nitropyrazole, potassium carbonate, ethyl iodide and 20 mL of N,N-dimethylaminocarboxamide into a three-necked bottle, reacting for 3 hours at normal temperature, adding 100 mL of ethyl acetate after the reaction is finished, washing the mixture for three times by saturated saline solution, drying an organic phase by using anhydrous Na₂SO₄, and purifying by column chromatography to obtain an intermediate 1-7 with the yield of 96%.

### Preparation of Compound 98-Compound 104

Compound 98 to compound 104 were prepared according to a similar method to that for compound 96.

### Preparation of Compound 105

Compound 105 was prepared similarly to compound 58, except that intermediates 1-6 in step vi were replaced with equimolar 3,5-dimethyl-4-nitropyrazole, specifically the preparation of intermediates 1-7: placing 3, 5-dimethyl-4-nitropyrazole, triethylamine and dichloromethane in sequence into a three-necked bottle, dropwise adding acetyl chloride (0.83 g) at 0° C., reacting at normal temperature for 3 h, adding 100 mL of ethyl acetate after the reaction is finished, washing with saturated saline solution for three times, drying an organic phase with anhydrous Na₂SO₄, and purifying by column chromatography to obtain intermediates 1-7 with the yield of 63%.

### Preparation of Compound 106-Compound 122

Compound 106-compound 122 were prepared according to a similar procedure as that for compound 105.

### Preparation of Compound 123

Compound 123 was prepared according to a similar manner to that of preparation 1, except that 1-(3,5-dichlorophenyl)-2,2,2-trifluoroethanone in step ii was replaced with an equimolar amount of 1-(3,5-dichloro-4-fluorophenyl)-2,2,2-trifluoroethanone, specifically, the preparation of intermediates 1-3 involved: sequentially taking 1-2 (10.00 g) of the intermediate, 1-(3,5-dichloro-4-fluorophenyl)-2,2,2-trifluoroethanone, 11.37 g of triethylamine and 100 mL of toluene into a three-neck flask, reacting for 16 h at 50° C., cooling to room temperature after the reaction is finished, performing suction filtration, and drying the solid to obtain 1-3 of the intermediate, wherein the yield is 93%.

### Preparation of Compound 124

Compound 124 was prepared analogously to preparation 2, except that 1-(3,5-dichlorophenyl)-2,2,2-trifluoroethanone in step ii was replaced by an equimolar amount of 1-(3,5-dichloro-4-fluorophenyl)-2,2,2-trifluoroethanone, and in particular intermediates 1-3 were prepared by: sequentially taking the intermediates 1-2, 1-(3,5-dichloro-4-fluorophenyl)-2,2,2-trifluoroethanone, triethylamine and 100 mL of toluene in a three-neck flask, reacting at 50° C. for 16 hours, cooling to room temperature after the reaction is finished, performing suction filtration, and drying the solid to obtain the intermediates 1-3 with the yield of 93%.

### Preparation of Compound 125-Compound 139

Compound 125-compound 139 was prepared according to a similar procedure to that used to prepare compound 124.

### Preparation of Compound 140

Compound 140 is prepared similarly to compound 58, except that 1-(3,5-dichlorophenyl)-2,2,2-trifluoroethanone in step ii is replaced with an equimolar amount of 1-(3,5-dichloro-4-fluorophenyl)-2,2,2-trifluoroethanone, specifically, the preparation of intermediates 1-3 comprises: sequentially taking the intermediates 1-2, 1-(3,5-dichloro-4-fluorophenyl)-2,2,2-trifluoroethanone, triethylamine and 100 mL of toluene in a three-neck flask, reacting at 50° C. for 16 hours, cooling to room temperature after the reaction is finished, performing suction filtration, and drying the solid to obtain the intermediates 1-3 with the yield of 93%.

### Preparation of Compound 141-Compound 157

Compounds 141-157 were prepared according to a similar procedure to that for compound 140.

### Preparation of Compound 158

Compound 158 is prepared analogously to compound 123 by replacing 4-aminopyrazole in step v with 3,5-dimethyl-1H-pyrazol-4-amine, specifically by placing intermediate 1-5 (0.50 g), CDI (0.39 g), DIPEA (0.56 g), and 10 mL of methylene chloride in a three-necked flask, adding 3,5-dimethyl-1H-pyrazol-4-amine, reacting for 4 h at room temperature, after completion of the reaction, washing 3 times with saturated NaHCO₃ solution, extracting the aqueous phase 3 times with 20 mL of methylene chloride, combining the organic phase after completion of the extraction, drying the organic phase with anhydrous Na₂SO₄, and purifying the organic phase by column chromatography to give compound 158 in 88% yield.

### Preparation of Compound 159

Compound 159 was prepared similarly to compound 124, except that intermediates 1-6 in step vi were replaced with equimolar 3,5-dimethyl-4-nitropyrazole, specifically the preparation of intermediates 1-7: 3,5-dimethyl-4-nitropyrazole, potassium carbonate (1.47 g), methyl iodide (1.50 g) and N,N-dimethylaminocarboxamide 20 mL are sequentially put into a three-necked bottle and reacted at normal temperature for 3 h, after the reaction is finished, 100 mL of ethyl acetate is added and washed with saturated saline three times, and then an organic phase is dried by anhydrous Na₂SO₄ and purified by column chromatography to obtain an intermediate 1-7 with the yield of 94%.

### Preparation of Compound 160-Compound 167

Compound 160 to compound 167 were prepared according to a similar method to that for compound 159.

### Preparation of Compound 168

Compound 168 is prepared similarly to compound 140, except that intermediates 1-6 in step vi are replaced with equimolar 3,5-dimethyl-4-nitropyrazole, specifically intermediates 1-7 are prepared by: placing 3,5-dimethyl-4-nitropyrazole, triethylamine and dichloromethane in a three-necked bottle in sequence, dropwise adding acetyl chloride at 0° C., reacting for 3 hours at normal temperature, adding 100 mL of ethyl acetate after the reaction is finished, washing with saturated saline solution for three times, drying an organic phase with anhydrous Na₂SO₄, and purifying by column chromatography to obtain an intermediate 1-7 with the yield of 68%.

### Preparation of Compound 169-Compound 185

Compound 169-compound 185 were prepared according to a similar procedure as that used to prepare compound 168.

### Test Example 1

Determining the toxicity activity of part of compounds on diamondback moth (Plutella xylostella) instar larvae by the leafdipping method.

The test agents of the compounds in Table 2 were weighed out accurately with an electronic analytical balance, and the original drug was dissolved in a 10000 ppm solution with dimethyl sulfoxide as the solvent and then diluted with 0.1% of Triton solution to 1 ppm and 0.25 ppm solutions. Taking the cabbage not contacted with the liquid medicine, and cutting the leaves into 6 cm-diameter circular leaves. Soaking the leaves in the prepared liquid medicine for 15 seconds, taking out and drying, and placing the leaves into 6 cm culture dishes, wherein 1 cabbage leaf is placed in each culture dish. The same standard second instar larvae were placed in petri dishes with 10 heads per dish and 4 replicates per concentration for a total of 40 heads. After inoculation, the culture dish is placed in a constant-temperature culture chamber with the temperature of 25±1° C. and the illumination period of 16 (D) to 8 (L) h, and the number of dead and live larvae is investigated after 48 h and 72 h. A blank and a control of compound K1 and compound K2 were also provided.

The mortality is calculated according to the following formula 1, then the corrected mortality is calculated according to the formula 2, and finally the classification is carried out according to the corrected mortality value and the rating standard shown in the table 2, and the specific results are shown in the tables 3 and 4.

mortality %=(number of dead insects/total number of insects)×100% Equation 1;

corrected mortality %=[(treatment-control mortality)/(1-control mortality)]×100% Equation 2.

### Test Example 2

The toxicity activity of part of the compounds on the fourth instar larvae of the rice leaf rollers (Cnaphalocrocis medinalis) is determined by the leafdipping method.

The test agents of the compounds in Table 2 were weighed out accurately with an electronic analytical balance, and the original drug was dissolved completely in dimethylsulfoxide to 10000 ppm solution and then diluted with 0.1% aqueous solution of Triton to 5 ppm solution. And (4) selecting rice plants which are planted in a laboratory and are not contacted with the liquid medicine, and cutting leaves of the rice plants to about 8 cm. Soaking the leaves in the prepared liquid medicine for 15 seconds, taking out and drying, wrapping the roots with absorbent cotton, and putting the wrapped roots into 9 cm culture dishes, wherein 3-5 rice leaves are placed in each culture dish. The water bamboo sections used as the feed for larvae are peeled off gently, and the four-instar larvae of the same standard are placed in culture dishes with 10 heads per dish and 4 repetitions per concentration for a total of 40 heads. After the inoculation, the culture dish is placed in a constant temperature culture room with the temperature of 26±1° C. and the illumination period of 16 (D): 8 (L) h, and the number of dead and live larvae is investigated after 48 h. A blank and a control of compound K1 and compound K2 were also provided.

The mortality was calculated according to the following formula 1, then the corrected mortality was calculated according to the following formula 2, and finally the rating criteria shown in table 2 were combined for ranking according to the corrected mortality value, and the specific results are shown in table 5.

mortality %=(number of dead insects/total number of insects)×100% Equation 1;

corrected mortality %=[(treatment-control mortality)/(1-control mortality)]×100% Equation 2.

TABLE 2

| grade | mortality |
|---|---|
| A | 100% |
| B | 90% to less than 100% |
| C | 80% to less than 90% |
| D | 70% to less than 80% |
| E | 50% to less than 70% |
| F | less than 50% |

TABLE 3

| Compound | mortality (1 ppm) | | Compound | mortality (1 ppm) | |
|---|---|---|---|---|---|
| number | 48 h | 72 h | number | 48 h | 72 h |
| 1 | E | D | 2 | C | A |
| 3 | D | B | 4 | E | E |
| 5 | B | A | 8 | E | C |
| 7 | / | C | 10 | E | A |
| 9 | / | C | 12 | E | E |
| 11 | E | A | 16 | / | D |
| 23 | A | A | 24 | / | E |
| 27 | E | D | 28 | C | A |
| 33 | / | E | 30 | / | E |
| 39 | / | E | 36 | / | E |
| 47 | / | E | 40 | E | E |
| 51 | / | E | 42 | / | E |
| 59 | / | E | 44 | / | E |
| 67 | / | B | 52 | / | E |
| 69 | / | E | 58 | / | E |
| 71 | / | D | 60 | / | E |
| 73 | / | E | 64 | / | E |
| 75 | B | B | 68 | / | E |
| 97 | / | C | 72 | / | E |
| 123 | B | A | 98 | / | E |
| 125 | A | A | 102 | / | E |
| 127 | A | A | 104 | / | E |
| 129 | A | A | 124 | C | A |
| 131 | C | A | 130 | B | A |
| 133 | C | A | 132 | E | A |
| 137 | / | C | 134 | / | D |
| 139 | / | E | 140 | / | E |
| 149 | A | A | 150 | C | A |
| 151 | / | D | 154 | C | A |
| 153 | D | B | 158 | E | B |
| 155 | / | E | 160 | E | D |
| 157 | B | A | 162 | E | A |
| 159 | / | B | 168 | / | B |
| 161 | / | B | 170 | / | C |
| 169 | / | C | 174 | / | D |
| 171 | / | C | Compound K1 | F | F |
| 177 | / | C | | | |

Note: “/” indicates that no test record was made.

TABLE 4

| Compound number | mortality(0.25 ppm)(48 h) |
|---|---|
| 127 | A |
| Compound K1 | F |
| Compound K2 | F |

TABLE 5

| Compound number | mortality(5 ppm)(48 h) |
|---|---|
| 127 | A |
| Compound K1 | F |
| Compound K2 | D |

The compound provided in the present invention can, at low concentrations, successfully kill insects, mites, nematodes and piercing-sucking insects.

In particular, the compound containing the pyrazole structure provided by the invention has excellent insecticidal activity on agricultural insects such as Plutella xylostella, Ostrinia furnacalis, Helicoverpa armigera, Chilo suppressalis, Cnaphalocrocis medinalis, Spodoptera frugiperda, Spodoptera exigua, Spodoptera litura, Bemisia tabaci, rice planthopper, aphid, thrips, flea beetle and the like.

Specifically, the lethality of the compound 127 containing a pyrazole structure to Plutella xylostella can reach 100% under the concentration of 0.25 ppm, and the effects of the control compounds K1 and K2 are both less than 50%. And the lethality of the compound 127 to Cnaphalocrocis medinalis at the concentration of 5 ppm is 100 percent, which is far better than that of the control compounds K1 and K2.

The preferred embodiments of the present invention have been described above in detail, but the present invention is not limited thereto. Within the scope of the technical idea of the invention, many simple modifications can be made to the technical solution of the invention, including various technical features being combined in any other suitable way, and these simple modifications and combinations should also be regarded as the disclosure of the invention, and all fall within the scope of the invention.

1-10. (canceled)

11. A compound having a pyrazole structure of the following formula (I): 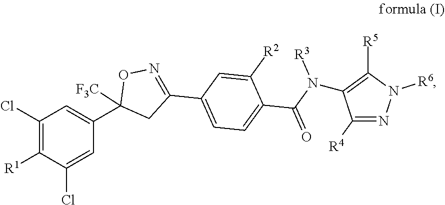
wherein, in the formula (I),
R¹ is selected from H, halogen, at least one halogen-substituted C₁₋₆ alkyl, at least one halogen-substituted C₁₋₆ alkoxy;
R² is selected from C₁₋₁₂ alkyl, C₁₋₁₂ alkoxy, halogen, at least one halogen-substituted C₁₋₆ alkyl, at least one halogen-substituted C₁₋₆ alkoxy and cyano;
R³ is selected from H, C₁₋₁₂ alkyl, C₁₋₁₂ alkoxy, C₃₋₁₂ cycloalkyl, —CO—R₃₁, —CO—CH₂—R₃₁; R³¹ is selected from C₁₋₁₂ alkyl, C₃₋₁₂ cycloalkyl and C₁₋₁₂ alkoxy;
R⁴, R⁵ are respectively and independently selected from H, C₁₋₁₂ alkyl, C₁₋₁₂ alkoxy, halogen, at least one halogen-substituted C₁₋₆ alkyl and at least one halogen-substituted C₁₋₆ alkoxy;
R⁶ is selected from the group consisting of H, C₁₋₁₂ alkyl unsubstituted or substituted by at least one group from combination A, C₁₋₁₂ alkoxy unsubstituted or substituted by at least one group from combination A, C₃₋₁₂ cycloalkyl unsubstituted or substituted by at least one group from combination A, phenyl unsubstituted or substituted by at least one group from combination A, C₃₋₁₂ cycloalkyl containing at least one hetero atom as a ring-forming atom, C₂₋₁₂ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂, R₆₁ is selected from the group consisting of C₃₋₁₂ cycloalkyl, C₃₋₁₂ cycloalkyl containing at least one heteroatom as a ring-forming atom, phenyl, R₆₂ is selected from the group consisting of C₁₋₁₂ alkyl, phenyl unsubstituted or substituted by at least one of group A, C₃₋₁₂ cycloalkyl, C₃₋₁₂ cycloalkyl containing at least one heteroatom as a ring-forming atom; R₆₃, R₆₄ are independently selected from C₁₋₁₂ alkyl; each of the heteroatoms is independently at least one selected from N, O, S; each n is independently 1 or 2; and
the combination A consists of hydroxyl, halogen, C₁₋₁₂ alkyl, C₁₋₁₂ alkoxy and trimethylsilyl,
or an agrochemically acceptable salt, a hydrate thereof, or a solvate thereof.

12. The compound, an agrochemically acceptable salt, a hydrate thereof, or a solvate thereof according to claim 11, wherein, in formula (I),
R¹ is selected from H, halogen, at least one halogen-substituted C₁₋₆ alkyl, at least one halogen-substituted C₁₋₆ alkoxy;
R² is selected from C₁₋₈ alkyl, C₁₋₈ alkoxy, halogen, at least one halogen-substituted C₁₋₆ alkyl, at least one halogen-substituted C₁₋₆ alkoxy and cyano;
R³ is selected from H, C₁₋₈ alkyl, C₁₋₈ alkoxy, C₃₋₁₀ cycloalkyl, —CO—R₃₁, —CO—CH₂—R₃₁; R₃₁ is selected from C₁₋₈ alkyl, C₃₋₁₀ cycloalkyl and C₁₋₈ alkoxy;
R⁴ and R⁵ are respectively and independently selected from H, C₁₋₈ alkyl, C₁₋₈ alkoxy, halogen, at least one halogen-substituted C₁₋₆ alkyl and at least one halogen-substituted C₁₋₆ alkoxy;
R⁶ is selected from the group consisting of H, C₁₋₁₀ alkyl unsubstituted or substituted by at least one group from combination A, C₁₋₁₀ alkoxy unsubstituted or substituted by at least one group from combination A, C₃₋₁₀ cycloalkyl unsubstituted or substituted by at least one group from combination A, phenyl unsubstituted or substituted by at least one group from combination A, C₃₋₁₀ cycloalkyl containing at least one heteroatom as a ring-forming atom, C₂₋₁₀ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂; R₆₁ is selected from the group consisting of C₃₋₁₀ cycloalkyl, C₃₋₁₀ cycloalkyl containing at least one heteroatom as a ring-forming atom, phenyl; R₆₂ is selected from the group consisting of C₁₋₁₀ alkyl, phenyl unsubstituted or substituted by at least one of the groups in combination A, C₃₋₁₀ cycloalkyl, C₃₋₁₀ cycloalkyl containing at least one heteroatom as a ring-forming atom; R₆₃ and R₆₄ are independently selected from C₁₋₁₀ alkyl of, each of the heteroatoms is independently at least one selected from N, O, S; each n is independently 1 or 2; and
the combination A consists of hydroxyl, halogen, C₁₋₈ alkyl, C₁₋₈ alkoxy and trimethylsilyl.

13. The compound, an agrochemically acceptable salt, a hydrate thereof, or a solvate thereof according to claim 12, wherein, in formula (I),
R¹ is selected from H, F, chloro, bromo;
R² is selected from methyl, ethyl, n-propyl, isopropyl, n-butyl, tert-butyl, cyano;
R³ is selected from the group consisting of H, methyl, ethyl, n-propyl, isopropyl, n-butyl, tert-butyl, methoxy, ethoxy, n-propoxy, isopropoxy, n-butoxy, tert-butoxy, cyclopropyl, cyclobutyl, cyclopentyl, cyclohexyl, —CO—R₃₁, —CO—CH₂—R₃₁; R₃₁ is selected from C₁₋₆ alkyl, C₃₋₆ cycloalkyl and C₁₋₆ alkoxy;
R⁴, R⁵ are each independently selected from H, methyl, ethyl, n-propyl, isopropyl, n-butyl, tert-butyl;
R⁶ is selected from the group consisting of H, C₁₋₁₀ alkyl unsubstituted or substituted by at least one group from combination A, C₁₋₁₀ alkoxy unsubstituted or substituted by at least one group from combination A, C₃₋₁₀ cycloalkyl unsubstituted or substituted by at least one group from combination A, phenyl unsubstituted or substituted by at least one group from combination A, C₃₋₁₀ cycloalkyl containing at least one heteroatom as a ring-forming atom, C₂₋₁₀ hydrocarbon containing at least one unsaturated carbon-carbon double bond or unsaturated carbon-carbon triple bond which is unsubstituted or substituted by at least one group from combination A, —(CH₂)ₙ—R₆₁, —CO—R₆₂, —COO—R₆₂, —CO—N(R₆₃R₆₄), —S(═O)₂—R₆₂, —(CH₂)ₙ—CO—R₆₂; R₆₁ is selected from the group consisting of C₃₋₁₀ cycloalkyl, C₃₋₁₀ cycloalkyl containing at least one heteroatom as a ring-forming atom, phenyl; R₆₂ is selected from the group consisting of C₁₋₁₀ alkyl, phenyl unsubstituted or substituted by at least one of the groups in combination A, C₃₋₁₀ cycloalkyl, C₃₋₁₀ cycloalkyl containing at least one heteroatom as a ring-forming atom; R₆₃ and R₆₄ are independently selected from C₁₋₁₀ alkyl; each of the heteroatoms is independently at least one selected from N, O, S; each n is independently 1 or 2; and
the combination A consists of hydroxyl, halogen, C₁₋₈ alkyl, C₁₋₈ alkoxy and trimethylsilyl.

14. The compound, an agrochemically acceptable salt, a hydrate thereof, or a solvate thereof according to claim 13, wherein the compound is selected from any one of the following:
Number A1: R¹ is H, R² is CH₃, R³ is H, R⁴ is H, R⁵ is H, R⁶ is —H;
Number A2: R¹ is H, R² is CH₃, R³ is H, R⁴ is H, R⁵ is H, R⁶ is —CH₃;
Number A3: R¹ is H, R² is CH₃, R³ is H, R⁴ is H, R⁵ is H, R⁶ is —CHF₂;
Number A4: R¹ is H, R² is CH₃, R³ is H, R⁴ is H, R⁵ is H, R⁶ is —CF₃;
Number A5: R¹ is H, R² is CH₃, R³ is H, R⁴ is H, R⁵ is H, R⁶ is —CH₂CH₃;
Number A6: R¹ is H, R² is CH₃, R³ is H, R⁴ is H, R⁵ is H, R⁶ is —CH₂CH₂CH₃;
Number A7: R¹ is H, R² is CH₃, R³ is H, R⁴ is H, R⁵ is H, R⁶ is —CH(CH₃)₂;
Number A8: R¹ is H, R² is CH₃, R³ is H, R⁴ is H, R⁵ is H, R⁶ is —CH₂CH₂OH;
Number A9: R¹ is H, R² is CH₃, R³ is H, R⁴ is H, R⁵ is H, R⁶ is —CH₂CF₃;
Number A10: R¹ is H, R² is CH₃, R³ is H, R⁴ is H, R⁵ is H, R⁶ is —CH₂CHF₂;
Number A11: R¹ is H, R² is CH₃, R³ is H, R⁴ is H, R⁵ is H, R⁶ is —CH₂CH₂F;
Number A12: R¹ is H, R² is CH₃, R³ is H, R⁴ is H, R⁵ is H, R⁶ is —CH₂CH₂CF₃;
Number A13: R¹ is H, R² is CH₃, R³ is H, R⁴ is H, R⁵ is H, R⁶ is —CH₂CH₂CH₂F;
Number A14: R¹ is H, R² is CH₃, R³ is H, R⁴ is H, R⁵ is H, R⁶ is —CH₂CH₂OCH₃;
Number A15: R¹ is H, R² is CH₃, R³ is H, R⁴ is H, R⁵ is H, R⁶ is —CH₂CH₂OCH₂CH₃;
Number A16: R¹ is H, R² is CH₃, R³ is H, R⁴ is H, R⁵ is H, R⁶ is —CH₂(CH₂)₂OCH₃;
Number A17: R¹ is H, R² is CH₃, R³ is H, R⁴ is H, R⁵ is H, R⁶ is —CH₂CH₂CH₂CH₃;
Number A18: R¹ is H, R² is CH₃, R³ is H, R⁴ is H, R⁵ is H, R⁶ is —CH(CH₃)CH₂CH₃;
Number A19: R¹ is H, R² is CH₃, R³ is H, R⁴ is H, R⁵ is H, R⁶ is —CH₂(CH₂)₃CH₃;
Number A20: R¹ is H, R² is CH₃, R³ is H, R⁴ is H, R⁵ is H, R⁶ is 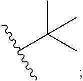 [SMILES: *C(C)(C)C]
Number A21: R¹ is H, R² is CH₃, R³ is H, R⁴ is H, R⁵ is H, R⁶ is 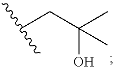 [SMILES: *CC(C)(C)O]
Number A22: R¹ is H, R² is CH₃, R³ is H, R⁴ is H, R⁵ is H, R⁶ is 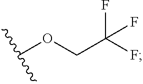 [SMILES: *OCC(F)(F)F]
Number A23: R¹ is H, R² is CH₃, R³ is H, R⁴ is H, R⁵ is H, R⁶ is 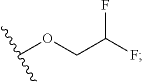 [SMILES: *OCC(F)F]
Number A24: R¹ is H, R² is CH₃, R³ is H, R⁴ is H, R⁵ is H, R⁶ is 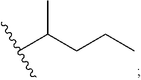 [SMILES: *C(C)CCC]
Number A25: R¹ is H, R² is CH₃, R³ is H, R⁴ is H, R⁵ is H, R⁶ is 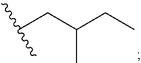 [SMILES: *CC(C)CC]
Number A26: R¹ is H, R² is CH₃, R³ is H, R⁴ is H, R⁵ is H, R⁶ is 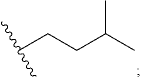 [SMILES: *CCC(C)C]
Number A27: R¹ is H, R² is CH₃, R³ is H, R⁴ is H, R⁵ is H, R⁶ is 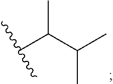 [SMILES: *C(C)C(C)C]
Number A28: R¹ is H, R² is CH₃, R³ is H, R⁴ is H, R⁵ is H, R⁶ is 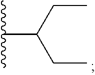 [SMILES: *C(CC)CC]
Number A29: R¹ is H, R² is CH₃, R³ is H, R⁴ is H, R⁵ is H, R⁶ is 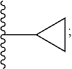 [SMILES: *C1CC1]
Number A30: R¹ is H, R² is CH₃, R³ is H, R⁴ is H, R⁵ is H, R⁶ is 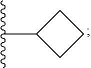 [SMILES: *C1CCC1]
Number A31: R¹ is H, R² is CH₃, R³ is H, R⁴ is H, R⁵ is H, R⁶ is 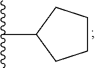 [SMILES: *C1CCCC1]
Number A32: R¹ is H, R² is CH₃, R³ is H, R⁴ is H, R⁵ is H, R⁶ is 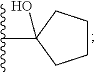 [SMILES: *C1(O)CCCC1]
Number A33: R¹ is H, R² is CH₃, R³ is H, R⁴ is H, R⁵ is H, R⁶ is 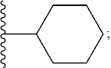 [SMILES: *C1CCCCC1]
Number A34: R¹ is H, R² is CH₃, R³ is H, R⁴ is H, R⁵ is H, R⁶ is 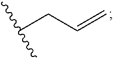 [SMILES: *CC=C]
Number A35: R¹ is H, R² is CH₃, R³ is H R⁴ is H R⁵ is H R⁶ is 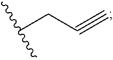 [SMILES: *CC#C]
Number A36: R¹ is H, R² is CH₃, R³ is H, R⁴ is H, R⁵ is H, R⁶ is 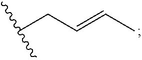 [SMILES: *C/C=C/C]
Number A37: R¹ is H, R² is CH₃, R³ is H, R⁴ is H, R⁵ is H, R⁶ is 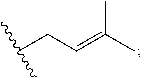 [SMILES: *CC=C(C)C]
Number A38: R¹ is H, R² is CH₃, R³ is H, R⁴ is H, R⁵ is H, R⁶ is 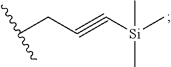 [SMILES: *CC#C[Si](C)(C)C]
Number A39: R¹ is H, R² is CH₃, R³ is H, R⁴ is H, R⁵ is H, R⁶ is 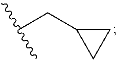 [SMILES: *CC1CC1]
Number A40: R¹ is H, R² is CH₃, R³ is H, R⁴ is H, R⁵ is H, R⁶ is 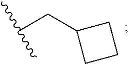 [SMILES: *CC1CCC1]
Number A41: R¹ is H, R² is CH₃, R³ is H, R⁴ is H, R⁵ is H, R⁶ is 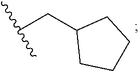 [SMILES: *CC1CCCC1]
Number A42: R¹ is H, R² is CH₃, R³ is H, R⁴ is H, R⁵ is H, R⁶ is 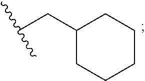 [SMILES: *CC1CCCCC1]
Number A43: R¹ is H, R² is CH₃, R³ is H, R⁴ is H, R⁵ is H, R⁶ is 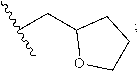 [SMILES: *CC1CCCO1]
Number A44: R¹ is H, R² is CH₃, R³ is H, R⁴ is H, R⁵ is H, R⁶ is 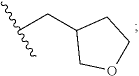 [SMILES: *CC1CCOC1]
Number A45: R¹ is H, R² is CH₃, R³ is H, R⁴ is H, R⁵ is H, R⁶ is 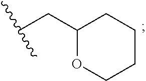 [SMILES: *CC1CCCCO1]
Number A46: R¹ is H, R² is CH₃, R³ is H, R⁴ is H, R⁵ is H, R⁶ is 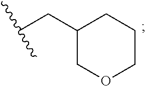 [SMILES: *CC1CCCOC1]
Number A47: R¹ is H, R² is CH₃, R³ is H, R⁴ is H, R⁵ is H, R⁶ is 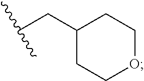 [SMILES: *CC1CCOCC1]
Number A48: R¹ is H, R² is CH₃, R³ is H, R⁴ is H, R⁵ is H, R⁶ is 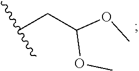 [SMILES: *CC(OC)OC]
Number A49: R¹ is H, R² is CH₃, R³ is H, R⁴ is H, R⁵ is H, R⁶ is 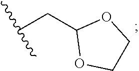 [SMILES: *CC1OCCO1]
Number A50: R¹ is H, R² is CH₃, R³ is H, R⁴ is H, R⁵ is H, R⁶ is 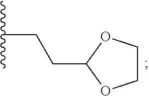 [SMILES: *CCC1OCCO1]
Number A51: R¹ is H, R² is CH₃, R³ is H, R⁴ is H, R⁵ is H, R⁶ is 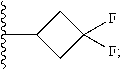 [SMILES: *C1CC(F)(F)C1]
Number A52: R¹ is H, R² is CH₃, R³ is H, R⁴ is H, R⁵ is H, R⁶ is 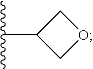 [SMILES: *C1COC1]
Number A53: R¹ is H, R² is CH₃, R³ is H, R⁴ is H, R⁵ is H, R⁶ is 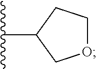 [SMILES: *C1CCOC1]
Number A54: R¹ is H, R² is CH₃, R³ is H, R⁴ is H, R⁵ is H, R⁶ is 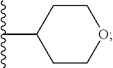 [SMILES: *C1CCOCC1]
Number A55: R¹ is H, R² is CH₃, R³ is H, R⁴ is H, R⁵ is H, R⁶ is 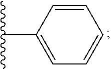 [SMILES: *c1ccccc1]
Number A56: R¹ is H, R² is CH₃, R³ is H, R⁴ is H, R⁵ is H, R⁶ is 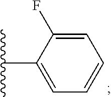 [SMILES: *c1ccccc1F]
Number A57: R¹ is H, R² is CH₃, R³ is H, R⁴ is H, R⁵ is H, R⁶ is 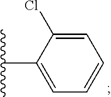 [SMILES: *c1ccccc1Cl]
Number A58: R¹ is H, R² is CH₃, R³ is H, R⁴ is H, R⁵ is H, R⁶ is 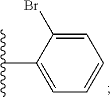 [SMILES: *c1ccccc1Br]
Number A59: R¹ is H, R² is CH₃, R³ is H, R⁴ is H, R⁵ is H, R⁶ is 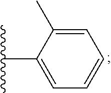 [SMILES: *c1ccccc1C ; ";"]
Number A60: R¹ is H, R² is CH₃, R³ is H, R⁴ is H, R⁵ is H, R⁶ is 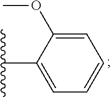 [SMILES: *c1ccccc1OC ; ";"]
Number A61: R¹ is H, R² is CH₃, R³ is H, R⁴ is H, R⁵ is H, R⁶ is 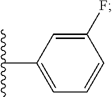 [SMILES: *c1cccc(F)c1]
Number A62: R¹ is H, R² is CH₃, R³ is H, R⁴ is H, R⁵ is H, R⁶ is 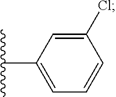 [SMILES: *c1cccc(Cl)c1]
Number A63: R¹ is H, R² is CH₃, R³ is H, R⁴ is H, R⁵ is H, R⁶ is 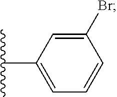 [SMILES: *c1cccc(Br)c1]
Number A64: R¹ is H, R² is CH₃, R³ is H R⁴ is H R⁵ is H R⁶ is 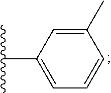 [SMILES: *c1cccc(C)c1 ; ";"]
Number A65: R¹ is H, R²H is C₃, R³ is H, R⁴ is H, R⁵ is H, R⁶ is 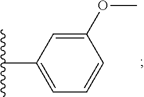 [SMILES: *c1cccc(OC)c1 ; ";"]
Number A66: R¹ is H, R² is CH₃, R³ is H, R⁴ is H, R⁵ is H, R⁶ is 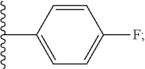 [SMILES: *c1ccc(F)cc1]
Number A67: R¹ is H, R² is CH₃, R³ is H, R⁴ is H, R⁵ is H, R⁶ is 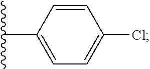 [SMILES: *c1ccc(Cl)cc1]
Number A68: R¹ is H, R² is CH₃, R³ is H, R⁴ is H, R⁵ is H, R⁶ is 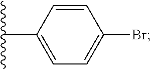 [SMILES: *c1ccc(Br)cc1]
Number A69: R¹ is H, R² is CH₃, R³ is H, R⁴ is H, R⁵ is H, R⁶ is 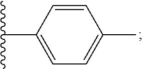 [SMILES: *c1ccc(C)cc1]
Number A70: R¹ is H, R² is CH₃, R³ is H, R⁴ is H, R⁵ is H, R⁶ is 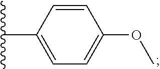 [SMILES: *c1ccc(OC)cc1]
Number A71: R¹ is H, R² is CH₃, R³ is H, R⁴ is H, R⁵ is H, R⁶ is 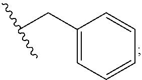 [SMILES: *Cc1ccccc1]
Number A72: R¹ is H, R² is CH₃, R³ is H, R⁴ is H, R⁵ is H, R⁶ is —COCH₃;
Number A73: R¹ is H, R² is CH₃, R³ is H, R⁴ is H, R⁵ is H, R⁶ is —COCH₂CH₃;
Number A74: R¹ is H, R² is CH₃, R³ is H, R⁴ is H, R⁵ is H, R⁶ is —COCH₂CH₂CH₃;
Number A75: R¹ is H, R² is CH₃, R³ is H, R⁴ is H, R⁵ is H, R⁶ is —COCH(CH₃)₂;
Number A76: R¹ is H, R² is CH₃, R³ is H, R⁴ is H, R⁵ is H, R⁶ is —COC₆H₅;
Number A77: R¹ is H, R² is CH₃, R³ is H, R⁴ is H, R⁵ is H, R⁶ is 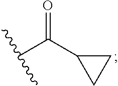 [SMILES: *C(=O)C1CC1]
Number A78: R¹ is H, R² is CH₃, R³ is H, R⁴ is H, R⁵ is H, R⁶ is 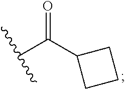 [SMILES: *C(=O)C1CCC1]
Number A79: R¹ is H, R² is CH₃, R³ is H, R⁴ is H, R⁵ is H, R⁶ is 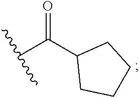 [SMILES: *C(=O)C1CCCC1]
Number A80: R¹ is H, R² is CH₃, R³ is H, R⁴ is H, R⁵ is H, R⁶ is 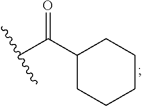 [SMILES: *C(=O)C1CCCCC1]
Number A81: R¹ is H, R² is CH₃, R³ is H, R⁴ is H, R⁵ is H, R⁶ is 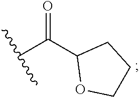 [SMILES: *C(=O)C1CCCO1]
Number A82: R¹ is H, R² is CH₃, R³ is H, R⁴ is H, R⁵ is H, R⁶ is 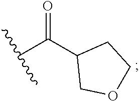 [SMILES: *C(=O)C1CCOC1]
Number A83: R¹ is H, R² is CH₃, R³ is H, R⁴ is H, R⁵ is H, R⁶ is 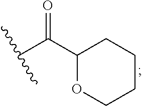 [SMILES: *C(=O)C1CCCCO1]
Number A84: R¹ is H, R² is CH₃, R³ is H, R⁴ is H, R⁵ is H, R⁶ is 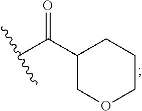 [SMILES: *C(=O)C1CCCOC1]
Number A85: R¹ is H, R² is CH₃, R³ is H, R⁴ is H, R⁵ is H, R⁶ is 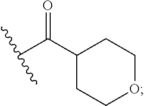 [SMILES: *C(=O)C1CCOCC1]
Number A86: R¹ is H, R² is CH₃, R³ is H, R⁴ is H, R⁵ is H, R⁶ is 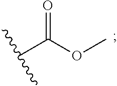 [SMILES: *C(=O)OC]
Number A87: R¹ is H, R² is CH₃, R³ is H, R⁴ is H, R⁵ is H, R⁶ is 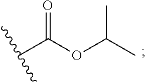 [SMILES: *C(=O)OC(C)C]
Number A88: R¹ is H, R² is CH₃, R³ is H, R⁴ is H, R₅ is H, R⁶ is 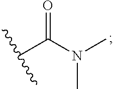 [SMILES: *C(=O)N(C)C]
Number A89: R¹ is H, R² is CH₃, R³ is H, R⁴ is H, R⁵ is H, R⁶ is 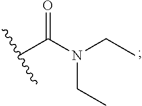 [SMILES: *C(=O)N(CC)CC]
Number A90: R¹ is H, R² is CH₃, R³ is H, R⁴ is H, R⁵ is H, R⁶ is —S(═O)₂CH₃;
Number A91: R¹ is H, R² is CH₃, R³ is H, R⁴ is H, R⁵ is H, R⁶ is —S(═O)₂CH₂CH₃,
Number A92: R¹ is H, R² is CH₃, R³ is H, R⁴ is H, R⁵ is H, R⁶ is —S(═O)₂CH₂CH₂CH₃;
Number A93: R¹ is H, R² is CH₃, R³ is H, R⁴ is H, R⁵ is H, R⁶ is —S(═O)₂CH(CH₃)₂;
Number A94: R¹ is H, R² is CH₃, R³ is H, R⁴ is H, R⁵ is H, R⁶ is —S(═O)₂C₆H₅;
Number A95: R¹ is H, R² is CH₃, R³ is H, R⁴ is H, R⁵ is H, R⁶ is 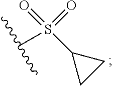 [SMILES: *S(=O)(=O)C1CC1]
Number A96: R¹ is H, R² is CH₃, R³ is H, R⁴ is H, R⁵ is H, R⁶ is 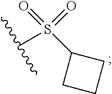 [SMILES: *S(=O)(=O)C1CCC1]
Number A97: R¹ is H, R² is CH₃, R³ is H, R⁴ is H, R⁵ is H, R⁶ is 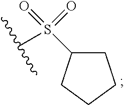 [SMILES: *S(=O)(=O)C1CCCC1]
Number A98: R¹ is H, R² is CH₃, R³ is H, R⁴ is H, R⁵ is H, R⁶ is 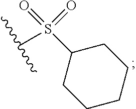 [SMILES: *S(=O)(=O)C1CCCCC1]
Number A99: R¹ is H, R² is CH₃, R³ is H, R⁴ is H, R⁵ is H, R⁶ is 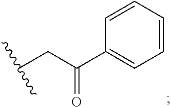 [SMILES: *CC(=O)c1ccccc1]
NumberA100: R¹ is H, R² is CH₃, R³ is H, R⁴ is H, R⁵ is H, R⁶ is 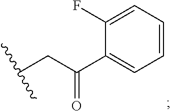 [SMILES: *CC(=O)c1ccccc1F]
NumberA101: R¹ is H, R² is CH₃, R³ is H, R⁴ is H, R⁵ is H, R⁶ is 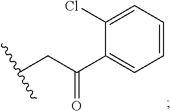 [SMILES: *CC(=O)c1ccccc1Cl]
NumberA102: R¹ is H, R² is CH₃, R³ is H, R⁴ is H, R⁵ is H, R⁶ is 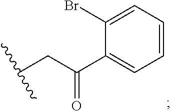 [SMILES: *CC(=O)c1ccccc1Br]
Number A103: R¹ is H, R² is CH₃, R³ is H, R⁴ is H, R⁵ is H, R⁶ is 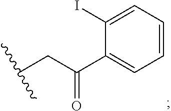 [SMILES: *CC(=O)c1ccccc1I]
Number A104: R¹ is H, R² is CH₃, R³ is H, R⁴ is H, R³ is H, R⁶ is 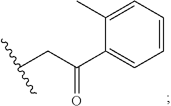 [SMILES: *CC(=O)c1ccccc1C]
Number A105: R¹ is H, R² is CH₃, R³ is H, R⁴ is H, R⁵ is H, R⁶ is 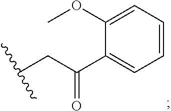 [SMILES: *CC(=O)c1ccccc1OC]
Number A106: R¹ is H, R² is CH₃, R³ is H, R⁴ is H, R⁵ is H, R⁶ is 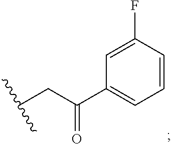 [SMILES: *CC(=O)c1cccc(F)c1]
Number A107: R¹ is H, R² is CH₃, R³ is H, R⁴ is H, R⁵ is H, R⁶ is 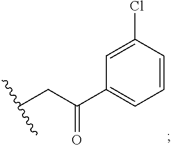 [SMILES: *CC(=O)c1cccc(Cl)c1]
Number A108: R¹ is H, R² is CH₃, R³ is H, R⁴ is H, R⁵ is H, R⁶ is 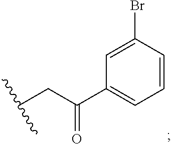 [SMILES: *CC(=O)c1cccc(Br)c1]
Number A109: R¹ is H, R² is CH₃, R³ is H, R⁴ is H, R⁵ is H, R⁶ is 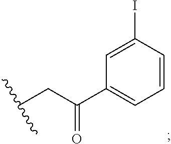 [SMILES: *CC(=O)c1cccc(I)c1]
Number A110: R¹ is H, R² is CH₃, R³ is H, R⁴ is H, R⁵ is H, R⁶ is 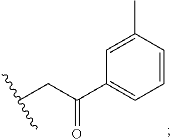 [SMILES: *CC(=O)c1cccc(C)c1]
Number A111: R¹ is H, R² is CH₃, R³ is H, R⁴ is H, R⁵ is H, R⁶ is 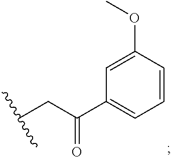 [SMILES: *CC(=O)c1cccc(OC)c1]
Number A112: R¹ is H, R² is CH₃, R³ is H, R⁴ is H, R⁵ is H, R⁶ is 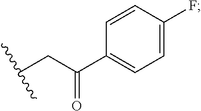 [SMILES: *CC(=O)c1ccc(F)cc1]
Number A113: R¹ is H, R² is CH₃, R³ is H, R⁴ is H, R⁵ is H, R⁶ is 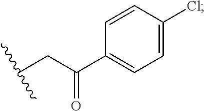 [SMILES: *CC(=O)c1ccc(Cl)cc1]
Number A114: R¹ is H, R² is CH₃, R³ is H, R⁴ is H, R⁵ is H, R⁶ is 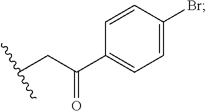 [SMILES: *CC(=O)c1ccc(Br)cc1]
Number A115: R¹ is H, R² is CH₃, R³ is H, R⁴ is H, R⁵ is H, R⁶ is 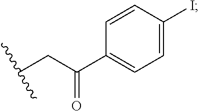 [SMILES: *CC(=O)c1ccc(I)cc1]
Number A116: R¹ is H, R² is CH₃, R³ is H, R⁴ is H, R⁵ is H, R⁶ is 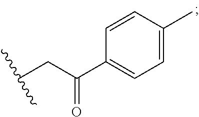 [SMILES: *CC(=O)c1ccc(C)cc1]
Number A117: R¹ is H, R² is CH₃, R³ is H, R⁴ is H, R⁵ is CH₃, R⁶ is 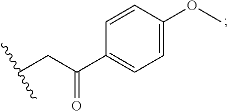 [SMILES: *CC(=O)c1ccc(OC)cc1]
Number A118: R¹ is H, R² is CH₃, R³ is H, R⁴ is H, R⁵ is CH₃, R⁶ is —H;
Number A119: R¹ is H, R² is CH₃, R³ is H, R⁴ is H, R⁵ is CH₃, R⁶ is —CH₃;
Number A120: R¹ is H, R² is CH₃, R³ is H, R⁴ is H, R⁵ is CH₃, R⁶ is —CH₂CH₃;
Number A121: R¹ is H, R² is CH₃, R³ is H, R⁴ is H, R⁵ is CH₃, R⁶ is —CH₂CH₂CH₃;
Number A122: R¹ is H, R² is CH₃, R³ is H, R⁴ is H, R⁵ is CH₃, R⁶ is —CH(CH₃)₂;
Number A123: R¹ is H, R² is CH₃, R³ is H, R⁴ is H, R⁵ is CH₃, R⁶ is 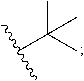 [SMILES: *C(C)(C)C ; ";"]
Number A124: R¹ is H, R² is CH₃, R³ is H, R⁴ is H, R⁵ is CH₃, R⁶ is 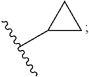 [SMILES: *C1CC1]
Number A125: R¹ is H, R² is CH₃, R³ is H, R⁴ is H, R⁵ is CH₃, R⁶ is 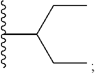 [SMILES: *C(CC)CC]
Number A126: R¹ is H, R² is CH₃, R³ is H, R⁴ is H, R⁵ is CH₃, R⁶ is 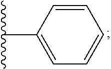 [SMILES: *c1ccccc1]
Number A127: R¹ is H, R² is CH₃, R³ is H, R⁴ is H, R⁵ is CH₃, R⁶ is 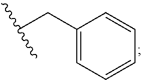 [SMILES: *Cc1ccccc1]
Number A128: R¹ is H, R² is CH₃, R³ is H, R⁴ is H, R⁵ is CH₃, R⁶ is —COCH₃,
Number A129: R¹ is H, R² is CH₃, R³ is H, R⁴ is H, R⁵ is CH₃, R⁶ is —COCH₂CH₃,
Number A130: R¹ is H, R² is CH₃, R³ is H, R⁴ is H, R⁵ is CH₃, R⁶ is —COCH₂CH₂CH₃;
Number A131: R¹ is H, R² is CH₃, R³ is H, R⁴ is H, R⁵ is CH₃, R⁶ is —COCH(CH₃)₂;
Number A132: R¹ is H, R² is CH₃, R³ is H, R⁴ is H, R⁵ is CH₃, R⁶ is —COC₆H₅;
Number A133: R¹ is H, R² is CH₃, R³ is H, R⁴ is H, R⁵ is CH₃, R⁶ is 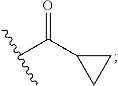 [SMILES: *C(=O)C1CC1]
Number A134: R¹ is H, R² is CH₃, R³ is H, R⁴ is H, R⁵ is CH₃, R⁶ is 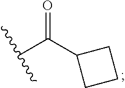 [SMILES: *C(=O)C1CCC1]
Number A135: R¹ is H, R² is CH₃, R³ is H, R⁴ is H, R⁵ is CH₃, R⁶ is 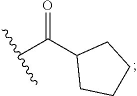 [SMILES: *C(=O)C1CCCC1]
Number A136: R¹ is H, R² is CH₃, R³ is H, R⁴ is H, R⁵ is CH₃, R⁶ is 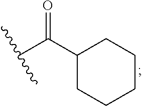 [SMILES: *C(=O)C1CCCCC1]
Number A137: R¹ is H, R² is CH₃, R³ is H, R⁴ is H, R⁵ is CH₃, R⁶ is 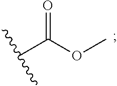 [SMILES: *C(=O)OC]
Number A138: R¹ is H, R² is CH₃, R³ is H, R⁴ is H, R⁵ is CH₃, R⁶ is 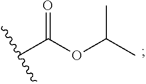 [SMILES: *C(=O)OC(C)C]
Number A139: R¹ is H, R² is CH₃, R³ is H, R⁴ is H, R⁵ is CH₃, R⁶ is 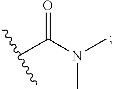 [SMILES: *C(=O)N(C)C]
Number A140: R¹ is H, R² is CH₃, R³ is H, R⁴ is H, R⁵ is CH₃, R⁶ is 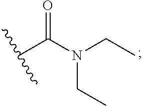 [SMILES: *C(=O)N(CC)CC]
Number A141: R¹ is H, R² is CH₃, R³ is H, R⁴ is H, R⁵ is CH₃, R⁶ is —S(═O)₂CH₃;
Number A142: R¹ is H, R² is CH₃, R³ is H, R⁴ is H, R⁵ is CH₃, R⁶ is —S(═O)₂CH₂CH₃;
Number A143: R¹ is H, R² is CH₃, R³ is H, R⁴ is H, R⁵ is CH₃, R⁶ is —S(═O)₂CH₂CH₂CH₃;
Number A144: R¹ is H, R² is CH₃, R³ is H, R⁴ is H, R⁵ is CH₃, R⁶ is —S(═O)₂CH(CH₃)₂;
Number A145: R¹ is H, R² is CH₃, R³ is H, R⁴ is H, R⁵ is CH₃, R⁶ is —S(═O)₂C₆H₅;
Number A146: R¹ is H, R² is CH₃, R³ is H, R⁴ is H, R⁵ is CH₃, R⁶ is 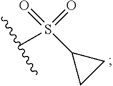 [SMILES: *S(=O)(=O)C1CC1]
Number A147: R¹ is H, R² is CH₃, R³ is H, R⁴ is H, R⁵ is CH₃, R⁶ is 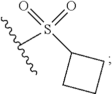 [SMILES: *S(=O)(=O)C1CCC1]
Number A148: R¹ is H, R² is CH₃, R³ is H, R⁴ is H, R⁵ is CH₃, R⁶ is 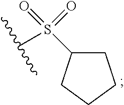 [SMILES: *S(=O)(=O)C1CCCC1]
Number A149: R¹ is H, R² is CH₃, R³ is H, R⁴ is H, R⁵ is CH₃, R⁶ is 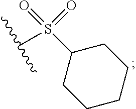 [SMILES: *S(=O)(=O)C1CCCCC1]
Number A150: R¹ is H, R² is CH₃, R³ is H, R⁴ is CH₃, R⁵ is CH₃, R⁶ is —H;
Number A151: R¹ is H, R² is CH₃, R³ is H, R⁴ is CH₃, R⁵ is CH₃, R⁶ is —CH₃;
Number A152: R¹ is H, R² is CH₃, R³ is H, R⁴ is CH₃, R⁵ is CH₃, R⁶ is —CH₂CH₃;
Number A153: R¹ is H, R² is CH₃, R³ is H, R⁴ is CH₃, R⁵ is CH₃, R⁶ is —CH₂CH₂CH₃;
Number A154: R¹ is H, R² is CH₃, R³ is H, R⁴ is CH₃, R⁵ is CH₃, R⁶ is —CH(CH₃)₂;
Number A155: R¹ is H, R² is CH₃, R³ is H, R⁴ is CH₃, R⁵ is CH₃, R⁶ is 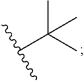 [SMILES: *C(C)(C)C ; ";"]
Number A156: R¹ is H, R² is CH₃, R³ is H, R⁴ is CH₃, R⁵ is CH₃, R⁶ is 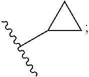 [SMILES: *C1CC1]
Number A157: R¹ is H, R² is CH₃, R³ is H R⁴ is CH₃, R⁵ is CH₃, R⁶ is 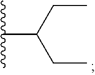 [SMILES: *C(CC)CC]
Number A158: R¹ is H, R² is CH₃, R³ is H, R⁴ is CH₃, R⁵ is CH₃, R⁶ is 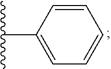 [SMILES: *c1ccccc1]
Number A159: R¹ is H, R² is CH₃, R³ is H, R⁴ is CH₃, R⁵ is CH₃, R⁶ is 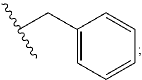 [SMILES: *Cc1ccccc1]
Number A160: R¹ is H, R² is CH₃, R³ is H, R⁴ is CH₃, R⁵ is CH₃, R⁶ is —COCH₃;
Number A161: R¹ is H, R² is CH₃, R³ is H, R⁴ is CH₃, R⁵ is CH₃, R⁶ is —COCH₂CH₃;
Number A162: R¹ is H, R² is CH₃, R³ is H, R⁴ is CH₃, R⁵ is CH₃, R⁶ is —COCH₂CH₂CH₃;
Number A163: R¹ is H, R² is CH₃, R³ is H, R⁴ is CH₃, R⁵ is CH₃, R⁶ is —COCH(CH₃)₂;
Number A164: R¹ is H, R² is CH₃, R³ is H, R⁴ is CH₃, R⁵ is CH₃, R⁶ is —COC₆H₅;
Number A165: R¹ is H, R² is CH₃, R³ is H, R⁴ is CH₃, R⁵ is CH₃, R⁶ is 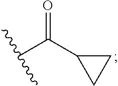 [SMILES: *C(=O)C1CC1]
Number A166: R¹ is H, R² is CH₃, R³ is H, R⁴ is CH₃, R⁵ is CH₃, R⁶ is 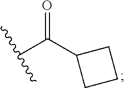 [SMILES: *C(=O)C1CCC1]
Number A167: R¹ is H, R² is CH₃, R³ is H, R⁴ is CH₃, R⁵ is CH₃, R⁶ is 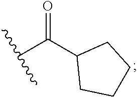 [SMILES: *C(=O)C1CCCC1]
Number A168: R¹ is H, R² is CH₃, R³ is H, R⁴ is CH₃, R⁵ is CH₃, R⁶ is 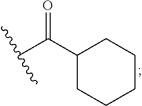 [SMILES: *C(=O)C1CCCCC1]
Number A169: R¹ is H, R² is CH₃, R³ is H, R⁴ is CH₃, R⁵ is CH₃, R⁶ is 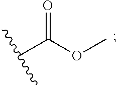 [SMILES: *C(=O)OC]
Number A170: R¹ is H, R² is CH₃, R³ is H, R⁴ is CH₃, R⁵ is CH₃, R⁶ is 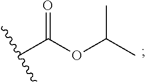 [SMILES: *C(=O)OC(C)C]
Number A171: R¹ is H, R² is CH₃, R³ is H, R⁴ is CH₃, R⁵ is CH₃, R⁶ is 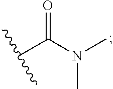 [SMILES: *C(=O)N(C)C]
Number A172: R¹ is H, R² is CH₃, R³ is H, R⁴ is CH₃, R⁵ is CH₃, R⁶ is 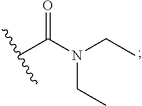 [SMILES: *C(=O)N(CC)CC]
Number A173: R¹ is H, R² is CH₃, R³ is H, R⁴ is CH₃, R⁵ is CH₃, R⁶ is —S(═O)₂CH₃;
Number A174: R¹ is H, R² is CH₃, R³ is H, R⁴ is CH₃, R⁵ is CH₃, R⁶ is —S(═O)₂CH₂CH₃;
Number A175: R¹ is H, R² is CH₃, R³ is H, R⁴ is CH₃, R⁵ is CH₃, R⁶ is —S(═O)₂CH₂CH₂CH₃;
Number A176: R¹ is H, R² is CH₃, R³ is H, R⁴ is CH₃, R⁵ is CH₃, R⁶ is —S(═O)₂CH(CH₃)₂;
Number A177: R¹ is H, R² is CH₃, R³ is H, R⁴ is CH₃, R⁵ is CH₃, R⁶ is —S(═O)₂C₆H₅;
Number A178: R¹ is H, R² is CH₃, R³ is H, R⁴ is CH₃, R⁵ is CH₃, R⁶ is 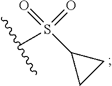 [SMILES: *S(=O)(=O)C1CC1]
Number A179: R¹ is H, R² is CH₃, R³ is H, R⁴ is CH₃, R⁵ is CH₃, R⁶ is 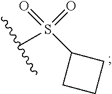 [SMILES: *S(=O)(=O)C1CCC1]
Number A180: R¹ is H, R² is CH₃, R³ is H, R⁴ is CH₃, R⁵ is CH₃, R⁶ is 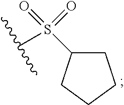 [SMILES: *S(=O)(=O)C1CCCC1]
Number A181: R¹ is H, R² is CH₃, R³ is H, R⁴ is CH₃, R⁵ is CH₃, R⁶ is 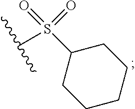 [SMILES: *S(=O)(=O)C1CCCCC1]
Number A182: R¹ is H, R² is CH₃, R³ is CH₃, R⁴ is H, R⁵ is H, R⁶ is 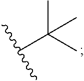 [SMILES: *C(C)(C)C ; ";"]
Number A183: R¹ is H, R² is CH₃, R³ is OCH₃, R⁴ is H, R⁵ is H, R⁶ is 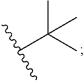 [SMILES: *C(C)(C)C ; ";"]
Number A184: R¹ is H, R² is CH₃, R³ is 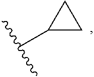 [SMILES: *C1CC1]
R⁴ is H, R⁵ is H, R⁶ is 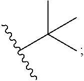 [SMILES: *C(C)(C)C ; ";"]
Number A185: R¹ is H, R² is CH₃, R³ is COCH₃, R⁴ is H, R⁵ is H, R⁶ is 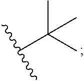 [SMILES: *C(C)(C)C ; ";"]
Number A186: R¹ is H, R² is CH₃, R³ is 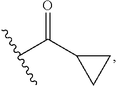 [SMILES: *C(=O)C1CC1]
R⁴ is H, R⁵ is H, R⁶ is 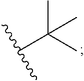 [SMILES: *C(C)(C)C ; ";"]
Number A187: R¹ is H, R² is CH₃, R³ is 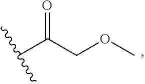 [SMILES: *C(=O)COC]
R⁴ is H, R⁵ is H, R⁶ is 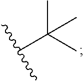 [SMILES: *C(C)(C)C ; ";"]
Number A188: R¹ is H, R² is CH₃, R³ is 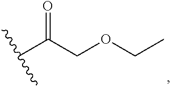 [SMILES: *C(=O)COCC]
R⁴ is H, R⁵ is H, R⁶ is 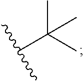 [SMILES: *C(C)(C)C ; ";"]
Number A189: R¹ is H, R² is CN, R³ is H, R⁴ is H, R⁵ is H, R⁶ is 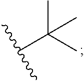 [SMILES: *C(C)(C)C ; ";"]
Number A190: R¹ is F, R² is CH₃, R³ is H, R⁴ is H, R⁵ is H, R⁶ is —H;
Number A191: R¹ is F, R² is CH₃, R³ is H, R⁴ is H, R⁵ is H, R⁶ is —CH₃;
Number A192: R¹ is F, R² is CH₃, R³ is H, R⁴ is H, R⁵ is H, R⁶ is —CH₂CH₃;
Number A193: R¹ is F, R² is CH₃, R³ is H, R⁴ is H, R⁵ is H, R⁶ is —CH₂CH₂CH₃;
Number A194: R¹ is F, R² is CH₃, R³ is H, R⁴ is H, R⁵ is H, R⁶ is —CH(CH₃)₂;
Number A195: R¹ is F, R² is CH₃, R³ is H, R⁴ is H, R⁵ is H, R⁶ is 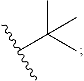 [SMILES: *C(C)(C)C ; ";"]
Number A196: R¹ is F, R² is CH₃, R³ is H, R⁴ is H, R⁵ is H, R⁶ is 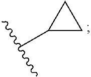 [SMILES: *C1CC1]
Number A197: R¹ is F, R² is CH₃, R³ is H, R⁴ is H, R⁵ is H, R⁶ is 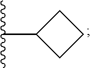 [SMILES: *C1CCC1]
Number A198: R¹ is F, R² is CH₃, R³ is H, R⁴ is H, R⁵ is H, R⁶ is 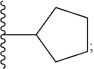 [SMILES: *C1CCCC1]
Number A199: R¹ is F, R² is CH₃, R³ is H, R⁴ is H, R⁵ is H, R⁶ is 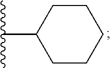 [SMILES: *C1CCCCC1]
Number A200: R¹ is F, R² is CH₃, R³ is H, R⁴ is H, R⁵ is H, R⁶ is 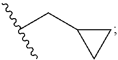 [SMILES: *CC1CC1]
Number A201: R¹ is F, R² is CH₃, R³ is H, R⁴ is H, R⁵ is H, R⁶ is 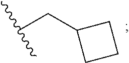 [SMILES: *CC1CCC1]
Number A202: R¹ is F, R² is CH₃, R³ is H, R⁴ is H, R⁵ is H, R⁶ is 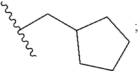 [SMILES: *CC1CCCC1]
Number A203: R¹ is F, R² is CH₃, R³ is H, R⁴ is H, R⁵ is H, R⁶ is 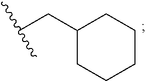 [SMILES: *CC1CCCCC1]
Number A204: R¹ is F, R² is CH₃, R³ is H, R⁴ is H, R⁵ is H, R⁶ is 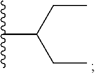 [SMILES: *C(CC)CC]
Number A205: R¹ is F, R² is CH₃, R³ is H, R⁴ is H, R⁵ is H, R⁶ is 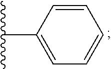 [SMILES: *c1ccccc1]
Number A206: R¹ is F, R² is CH₃, R³ is H, R⁴ is H, R⁵ is H, R⁶ is 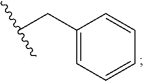 [SMILES: *Cc1ccccc1]
Number A207: R¹ is F, R² is CH₃, R³ is H, R⁴ is H, R⁵ is H, R⁶ is —COCH₃;
Number A208: R¹ is F, R² is CH₃, R³ is H, R⁴ is H, R⁵ is H, R⁶ is —COCH₂CH₃;
Number A209: R¹ is F, R² is CH₃, R³ is H, R⁴ is H, R⁵ is H, R⁶ is —COCH₂CH₂CH₃;
Number A210: R¹ is F, R² is CH₃, R³ is H, R⁴ is H, R⁵ is H, R⁶ is —COCH(CH₃)₂;
Number A211: R¹ is F, R² is CH₃, R³ is H, R⁴ is H, R⁵ is H, R⁶ is —COC₆H₅;
Number A212: R¹ is F, R² is CH₃, R³ is H, R⁴ is H, R⁵ is H, R⁶ is 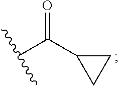 [SMILES: *C(=O)C1CC1]
Number A213: R¹ is F, R² is CH₃, R³ is H, R⁴ is H, R⁵ is H, R⁶ is 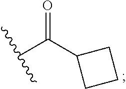 [SMILES: *C(=O)C1CCC1]
Number A214: R¹ is F, R² is CH₃, R³ is H, R⁴ is H, R⁵ is H, R⁶ is 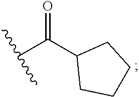 [SMILES: *C(=O)C1CCCC1]
Number A215: R¹ is F, R² is CH₃, R³ is H, R⁴ is H, R⁵ is H, R⁶ is 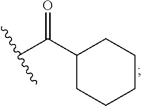 [SMILES: *C(=O)C1CCCCC1]
Number A216: R¹ is F, R² is CH₃, R³ is H, R⁴ is H, R⁵ is H, R⁶ is 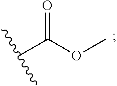 [SMILES: *C(=O)OC]
Number A217: R¹ is F, R² is CH₃, R³ is H, R⁴ is H, R⁵ is H, R⁶ is 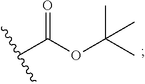 [SMILES: *C(=O)OC(C)(C)C]
Number A218: R¹ is F, R² is CH₃, R³ is H, R⁴ is H, R⁵ is H, R⁶ is 1 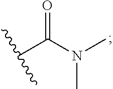 [SMILES: *C(=O)N(C)C]
Number A219: R¹ is F, R² is CH₃, R³ is H, R⁴ is H, R⁵ is H, R⁶ is 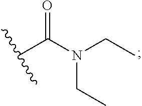 [SMILES: *C(=O)N(CC)CC]
Number A220: R¹ is F, R² is CH₃, R³ is H, R⁴ is H, R⁵ is H, R⁶ is —S(═O)₂CH₃;
Number A221: R¹ is F, R² is CH₃, R³ is H, R⁴ is H, R⁵ is H, R⁶ is —S(═O)₂CH₂CH₃;
Number A222: R¹ is F, R² is CH₃, R³ is H, R⁴ is H, R⁵ is H, R⁶ is —S(═O)₂CH₂CH₂CH₃;
Number A223: R¹ is F, R² is CH₃, R³ is H, R⁴ is H, R⁵ is H, R⁶ is —S(═O)₂CH(CH₃)₂;
Number A224: R¹ is F, R² is CH₃, R³ is H, R⁴ is H, R⁵ is H, R⁶ is —S(═O)₂C₆H₅;
Number A225: R¹ is F, R² is CH₃, R³ is H, R⁴ is H, R⁵ is H, R⁶ is 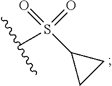 [SMILES: *S(=O)(=O)C1CC1]
Number A226: R¹ is F, R² is CH₃, R³ is H, R⁴ is H, R⁵ is H, R⁶ is 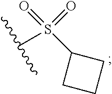 [SMILES: *S(=O)(=O)C1CCC1]
Number A227: R¹ is F, R² is CH₃, R³ is H, R⁴ is H, R⁵ is H, R⁶ is 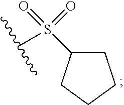 [SMILES: *S(=O)(=O)C1CCCC1]
Number A228: R¹ is F, R² is CH₃, R³ is H, R⁴ is H, R⁵ is H, R⁶ is 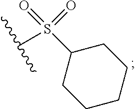 [SMILES: *S(=O)(=O)C1CCCCC1]
Number A229: R¹ is F, R² is CH₃, R³ is H, R⁴ is H, R⁵ is CH₃, R⁶ is —H;
Number A230: R¹ is F, R² is CH₃, R³ is H, R⁴ is H, R⁵ is CH₃, R⁶ is —CH₃;
Number A231: R¹ is F, R² is CH₃, R³ is H, R⁴ is H, R⁵ is CH₃, R⁶ is —CH₂CH₃;
Number A232: R¹ is F, R² is CH₃, R³ is H, R⁴ is H, R⁵ is CH₃, R⁶ is —CH₂CH₂CH₃;
Number A233: R¹ is F, R² is CH₃, R³ is H, R⁴ is H, R⁵ is CH₃, R⁶ is —CH(CH₃)₂;
Number A234: R¹ is F, R² is CH₃, R³ is H, R⁴ is H, R⁵ is CH₃, R⁶ is 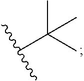 [SMILES: *C(C)(C)C ; ";"]
Number A235: R¹ is F, R² is CH₃, R³ is H R⁴ is H R⁵ is CH₃, R⁶ is 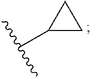 [SMILES: *C1CC1]
Number A236: R¹ is F, R² is CH₃, R³ is H, R⁴ is H, R⁵ is CH₃, R⁶ is 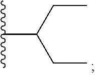 [SMILES: *C(CC)CC]
Number A237: R¹ is F, R² is CH₃, R³ is H, R⁴ is H, R⁵ is CH₃, R⁶ is 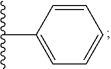 [SMILES: *c1ccccc1]
Number A238: R¹ is F, R² is CH₃, R³ is H, R⁴ is H, R⁵ is CH₃, R⁶ is 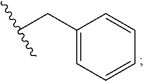 [SMILES: *Cc1ccccc1]
Number A239: R¹ is F, R² is CH₃, R³ is H, R⁴ is H, R⁵ is CH₃, R⁶ is —COCH₃;
Number A240: R¹ is F, R² is CH₃, R³ is H, R⁴ is H, R⁵ is CH₃, R⁶ is —COCH₂CH₃;
Number A241: R¹ is F, R² is CH₃, R³ is H, R⁴ is H, R⁵ is CH₃, R⁶ is —COCH₂CH₂CH₃;
Number A242: R¹ is F, R² is CH₃, R³ is H, R⁴ is H, R⁵ is CH₃, R⁶ is —COCH(CH₃)₂;
Number A243: R¹ is F, R² is CH₃, R³ is H, R⁴ is H, R⁵ is CH₃, R⁶ is —COC₆H₅;
Number A244: R¹ is F, R² is CH₃, R³ is H, R⁴ is H, R⁵ is CH₃, R⁶ is 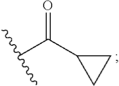 [SMILES: *C(=O)C1CC1]
Number A245: R¹ is F, R² is CH₃, R³ is H, R⁴ is H, R⁵ is CH₃, R⁶ is 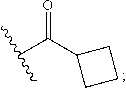 [SMILES: *C(=O)C1CCC1]
Number A246: R¹ is F, R² is CH₃, R³ is H, R⁴ is H, R⁵ is CH₃, R⁶ is 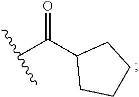 [SMILES: *C(=O)C1CCCC1]
Number A247: R¹ is F, R² is CH₃, R³ is H, R⁴ is H, R⁵ is CH₃, R⁶ is 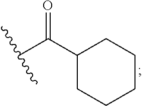 [SMILES: *C(=O)C1CCCCC1]
Number A248: R¹ is F, R² is CH₃, R³ is H, R⁴ is H, R⁵ is CH₃, R⁶ is 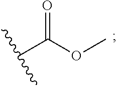 [SMILES: *C(=O)OC]
Number A249: R¹ is F, R² is CH₃, R³ is H, R⁴ is H, R⁵ is CH₃, R⁶ is 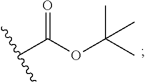 [SMILES: *C(=O)OC(C)(C)C]
Number A250: R¹ is F, R² is CH₃, R³ is H, R⁴ is H, R⁵ is CH₃, R⁶ is 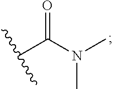 [SMILES: *C(=O)N(C)C]
Number A251: R¹ is F, R² is CH₃, R³ is H, R⁴ is H, R⁵ is CH₃, R⁶ is 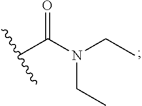 [SMILES: *C(=O)N(CC)CC]
Number A252: R¹ is F, R² is CH₃, R³ is H, R⁴ is H, R⁵ is CH₃, R⁶ is —S(═O)₂CH₃;
Number A253: R¹ is F, R² is CH₃, R³ is H, R⁴ is H, R⁵ is CH₃, R⁶ is —S(═O)₂CH₂CH₃;
Number A254: R¹ is F, R² is CH₃, R³ is H, R⁴ is H, R⁵ is CH₃, R⁶ is —S(═O)₂CH₂CH₂CH₃;
Number A255: R¹ is F, R² is CH₃, R³ is H, R⁴ is H, R⁵ is CH₃, R⁶ is —S(═O)₂CH(CH₃)₂;
Number A256: R¹ is F, R² is CH₃, R³ is H, R⁴ is H, R⁵ is CH₃, R⁶ is —S(═O)₂C₆H₅;
Number A257: R¹ is F, R² is CH₃, R³ is H, R⁴ is H, R⁵ is CH₃, R⁶ is 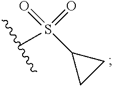 [SMILES: *S(=O)(=O)C1CC1]
Number A258: R¹ is F, R² is CH₃, R³ is H, R⁴ is H, R⁵ is CH₃, R⁶ is 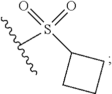 [SMILES: *S(=O)(=O)C1CCC1]
Number A259: R¹ is F, R² is CH₃, R³ is H, R⁴ is H, R⁵ is CH₃, R⁶ is 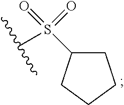 [SMILES: *S(=O)(=O)C1CCCC1]
Number A260: R¹ is F, R² is CH₃, R³ is H, R⁴ is H, R⁵ is CH₃, R⁶ is 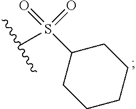 [SMILES: *S(=O)(=O)C1CCCCC1]
Number A261: R¹ is F, R² is CH₃, R³ is H, R⁴ is CH₃, R⁵ is CH₃, R⁶ is —H;
Number A262: R¹ is F, R² is CH₃, R³ is H, R⁴ is CH₃, R⁵ is CH₃, R⁶ is —CH₃;
Number A263: R¹ is F, R² is CH₃, R³ is H, R⁴ is CH₃, R⁵ is CH₃, R⁶ is —CH₂CH₃;
Number A264: R¹ is F, R² is CH₃, R³ is H, R⁴ is CH₃, R⁵ is CH₃, R⁶ is —CH₂CH₂CH₃;
Number A265: R¹ is F, R² is CH₃, R³ is H, R⁴ is CH₃, R⁵ is CH₃, R⁶ is —CH(CH₃)₂;
Number A266: R¹ is F, R² is CH₃, R³ is H, R⁴ is CH₃, R⁵ is CH₃, R⁶ is 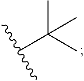 [SMILES: *C(C)(C)C ; ";"]
Number A267: R¹ is F, R² is CH₃, R³ is H, R⁴ is CH₃, R⁵ is CH₃, R⁶ is 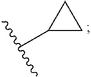 [SMILES: *C1CC1]
Number A268: R¹ is F, R² is CH₃, R³ is H, R⁴ is CH₃, R⁵ is CH₃, R⁶ is 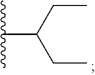 [SMILES: *C(CC)CC]
Number A269: R¹ is F, R² is CH₃, R³ is H, R⁴ is CH₃, R⁵ is CH₃, R⁶ is 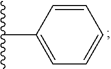 [SMILES: *c1ccccc1]
Number A270: R¹ is F, R² is CH₃, R³ is H, R⁴ is CH₃, R⁵ is CH₃, R⁶ is 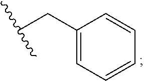 [SMILES: *Cc1ccccc1]
Number A271: R¹ is F, R² is CH₃, R³ is H, R⁴ is CH₃, R⁵ is CH₃, R⁶ is —COCH₃;
Number A272: R¹ is F, R² is CH₃, R³ is H, R⁴ is CH₃, R⁵ is CH₃, R⁶ is —COCH₂CH₃;
Number A273: R¹ is F, R² is CH₃, R³ is H, R⁴ is CH₃, R⁵ is CH₃, R⁶ is —COCH₂CH₂CH₃;
Number A274: R¹ is F, R² is CH₃, R³ is H, R⁴ is CH₃, R⁵ is CH₃, R⁶ is —COCH(CH₃)₂;
Number A275: R¹ is F, R² is CH₃, R³ is H, R⁴ is CH₃, R⁵ is CH₃, R⁶ is —COC₆H₅;
Number A276: R¹ is F, R² is CH₃, R³ is H, R⁴ is CH₃, R⁵ is CH₃, R⁶ is 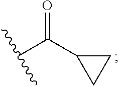 [SMILES: *C(=O)C1CC1]
Number A277: R¹ is F, R² is CH₃, R³ is H, R⁴ is CH₃, R⁵ is CH₃, R⁶ is 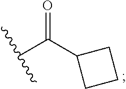 [SMILES: *C(=O)C1CCC1]
Number A278: R¹ is F, R² is CH₃, R³ is H, R⁴ is CH₃, R⁵ is CH₃, R⁶ is 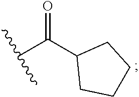 [SMILES: *C(=O)C1CCCC1]
Number A279: R¹ is F, R² is CH₃, R³ is H, R⁴ is CH₃, R⁵ is CH₃, R⁶ is 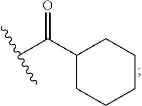 [SMILES: *C(=O)C1CCCCC1]
Number A280: R¹ is F, R² is CH₃, R³ is H, R⁴ is CH₃, R⁵ is CH₃, R⁶ is 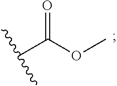 [SMILES: *C(=O)OC]
Number A281: R¹ is F, R² is CH₃, R³ is H, R⁴ is CH₃, R⁵ is CH₃, R⁶ is 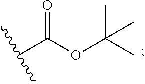 [SMILES: *C(=O)OC(C)(C)C]
Number A282: R¹ is F, R² is CH₃, R³ is H, R⁴ is CH₃, R⁵ is CH₃, R⁶ is 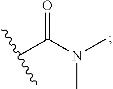 [SMILES: *C(=O)N(C)C]
Number A283: R¹ is F, R² is CH₃, R³ is H, R⁴ is CH₃, R⁵ is CH₃, R⁶ is 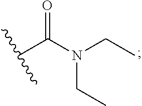 [SMILES: *C(=O)N(CC)CC]
Number A284: R¹ is F, R² is CH₃, R³ is H, R⁴ is CH₃, R⁵ is CH₃, R⁶ is —S(═O)₂CH₃;
Number A285: R¹ is F, R² is CH₃, R³ is H, R⁴ is CH₃, R⁵ is CH₃, R⁶ is —S(═O)₂CH₂CH₃;
Number A286: R¹ is F, R² is CH₃, R³ is H, R⁴ is CH₃, R⁵ is CH₃, R⁶ is —S(═O)₂CH₂CH₂CH₃;
Number A287: R¹ is F, R² is CH₃, R³ is H, R⁴ is CH₃, R⁵ is CH₃, R⁶ is —S(═O)₂CH(CH₃)₂;
Number A288: R¹ is F, R² is CH₃, R³ is H, R⁴ is CH₃, R⁵ is CH₃, R⁶ is —S(═O)₂C₆H₅;
Number A289: R¹ is F, R² is CH₃, R³ is H, R⁴ is CH₃, R⁵ is CH₃, R⁶ is 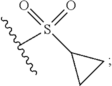 [SMILES: *S(=O)(=O)C1CC1]
Number A290: R¹ is F, R² is CH₃, R³ is H, R⁴ is CH₃, R⁵ is CH₃, R⁶ is 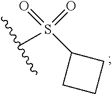 [SMILES: *S(=O)(=O)C1CCC1]
Number A291: R¹ is F, R² is CH₃, R³ is H, R⁴ is CH₃, R⁵ is CH₃, R⁶ is 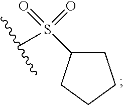 [SMILES: *S(=O)(=O)C1CCCC1]
Number A292: R¹ is F, R² is CH₃, R³ is CH₃, R⁴ is H, R⁵ is H, R⁶ is 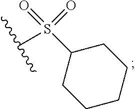 [SMILES: *S(=O)(=O)C1CCCCC1]
Number A293: R¹ is F, R² is CH₃, R³ is CH₃, R⁴ is H, R⁵ is H, R⁶ is 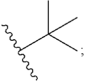 [SMILES: *C(C)(C)C ; ";"]
Number A294: R¹ is F, R² is CH₃, R³ is OCH₃, R⁴ is H, R⁵ is H, R⁶ is 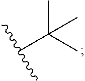 [SMILES: *C(C)(C)C ; ";"]
Number A295: R¹ is F, R² is CH₃, R³ is 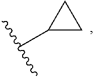 [SMILES: *C1CC1]
R⁴ is H, R⁵ is H, R⁶ is 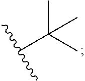 [SMILES: *C(C)(C)C ; ";"]
Number A296: R¹ is F, R² is CH₃, R³ is COCH₃, R⁴ is H, R⁵ is H, R⁶ is 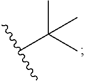 [SMILES: *C(C)(C)C ; ";"]
Number A297: R¹ is F, R² is CH₃, R³ is 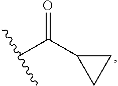 [SMILES: *C(=O)C1CC1]
R⁴ is H, R⁵ is H, R⁶ is 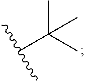 [SMILES: *C(C)(C)C ; ";"]
Number A298: R¹ is F, R² is CH₃, R³ is 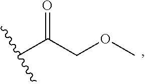 [SMILES: *C(=O)COC]
R⁴ is H, R⁵ is H, R⁶ is 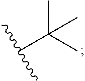 [SMILES: *C(C)(C)C ; ";"]
Number A299: R¹ is F, R² is CH₃, R³ is 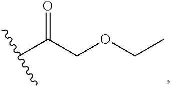 [SMILES: *C(=O)COCC]
R⁴ is H, R⁵ is H, R⁶ is 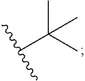 [SMILES: *C(C)(C)C ; ";"]
Number A300: R¹ is F, R² is CN, R³ is H, R⁴ is H, R⁵ is H, R⁶ is 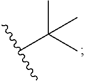 [SMILES: *C(C)(C)C ; ";"]
or
Number A301: R¹ is H, R² is CH₃, R³ is H, R⁴ is H, R⁵ is H, R⁶ is —CH₂OCH₃.

15. A method selected from insect killing, mite killing, nematode killing, piercing-sucking insects killing, or a combination thereof, comprising applying the compound, a agrochemically acceptable salt, a hydrate thereof, or a solvate thereof according to claim 11, to an inset, nematode, mite, piercing-sucking insect, or habitats thereof.

16. A insecticide which comprises, as an active ingredient, a pesticidally effective amount of the compound, an agrochemically acceptable salt, hydrate, or a solvate thereof according to claim 11.

17. A insecticide which comprises, as an active ingredient, a pesticidally effective amount of the compound, an agrochemically acceptable salt, hydrate, or a solvate thereof according to claim 12.

18. A insecticide which comprises, as an active ingredient, a pesticidally effective amount of the compound, an agrochemically acceptable salt, hydrate, or a solvate thereof according to claim 13.

19. A insecticide which comprises, as an active ingredient, a pesticidally effective amount of the compound, an agrochemically acceptable salt, hydrate, or a solvate thereof according to claim 14.

20. The insecticide according to claim 16, wherein the active ingredient is present in the insecticide in an amount of 1 to 99.9% by weight.

21. The insecticide of claim 20, wherein the active ingredient is present in the insecticide in an amount of 5 to 95% by weight.

22. The insecticide according to claim 16, wherein the insecticide is in a form selected from the group consisting of an emulsifiable concentrate, suspension, wettable powder, dustpowder, granules, aqueous solution, poison bait, mother liquor, mother powder, and combinations thereof.

23. The insecticide according to claim 17, wherein the insecticide is in a form selected from the group consisting of an emulsifiable concentrate, suspension, wettable powder, dustpowder, granules, aqueous solution, poison bait, mother liquor, mother powder, and combinations thereof.

24. The insecticide according to claim 18, wherein the insecticide is in a form selected from the group consisting of an emulsifiable concentrate, suspension, wettable powder, dustpowder, granules, aqueous solution, poison bait, mother liquor, mother powder, and combinations thereof.

25. The insecticide according to claim 19, wherein the insecticide is in a form selected from the group consisting of an emulsifiable concentrate, suspension, wettable powder, dustpowder, granules, aqueous solution, poison bait, mother liquor, mother powder, and combinations thereof.

26. The insecticide according to claim 20, wherein the insecticide is in a form selected from the group consisting of an emulsifiable concentrate, suspension, wettable powder, dustpowder, granules, aqueous solution, poison bait, mother liquor, mother powder, and combinations thereof.

27. The insecticide according to claim 21, wherein the insecticide is in a form selected from the group consisting of an emulsifiable concentrate, suspension, wettable powder, dustpowder, granules, aqueous solution, poison bait, mother liquor, mother powder, and combinations thereof.
